(19) United States
(12) Patent Application Publication
(10) Pub. No.: US 2024/0252098 A1
(43) Pub. Date: Aug. 1, 2024

(54) Neurosurgical Methods And Systems For Detecting And Removing Tumorous Tissue

(71) Applicant: Stryker European Operations Limited, Carrigtwohill (IE)

(72) Inventors: Kevin Buckley, Bishopstown (IE); Gerard W. Nunan, Ballincollig (IE)

(73) Assignee: Stryker European Operations Limited, Carrigtwohill (IE)

(21) Appl. No.: 18/603,982

(22) Filed: Mar. 13, 2024

Related U.S. Application Data
(63) Continuation-in-part of application No. PCT/IB2023/059198, filed on Sep. 15, 2023.
(60) Provisional application No. 63/406,966, filed on Sep. 15, 2022.

(51) Int. Cl. A61B 5/00; A61B 17/00; A61B 17/22

(52) U.S. Cl. CPC A61B 5/4064; A61B 5/0071; A61B 17/22012; A61B 2017/00057

(57) ABSTRACT

A surgical method of assessing tumor margins of obscured tissue of a patient who was administered a fluorescing agent is provided. The method includes creating an access hole through a tissue of the patient and extending to a surgical site of the patient, accessing the surgical site through the access hole and resecting tissue at the surgical site of the patient with a surgical instrument to create a resection cavity with at least a portion of thereof being outside of the field of view of the surgical microscope and including the obscured tissue, positioning the surgical instrument in contact with the obscured tissue, collecting fluorescent emission from the obscured tissue with the surgical instrument, and controlling an indicator of a tissue detection system based on the fluorescent emissions from the obscured tissue as collected by the surgical instrument.

### CROSS-REFERENCE TO RELATED APPLICATIONS

The subject patent application is a Continuation-in-Part of International Application No. PCT/IB2023/059198, filed on Sep. 15, 2023, which claims priority to, and all the benefits of, U.S. Provisional Patent Application No. 63/406,966, filed on Sep. 15, 2022, the entire contents of which are incorporated by reference herein.

### BACKGROUND

Glioma tumors may start in the glial cells of the brain or the spine. A surgical procedure, more specifically tumor resection, is often performed to resect the tumor. The goal of a surgical procedure for tumor resection is to achieve gross total resection (GTR). A very aggressive form of glioma is glioblastoma. In patients with glioblastoma, GTR has been shown to prolong the life of a patient by about 40% (e.g., from 10 months to 14 months). In patients with lower-grade gliomas, GTR increases the overall chances of survival.

5-Aminolevulinic Acid (5-ALA) is often given to patients a couple hours before surgery. 5-ALA is a compound that occurs naturally in the hemoglobin synthesis pathway. In cancer cells, the hemoglobin synthesis is disrupted and the pathway stalls at an intermediate compound called Protoporphyrin IX (PPIX). During surgery, the healthcare professional may illuminate an area of brain tissue with excitation light (i.e., blue light) from a surgical microscope. The surgery may be carried out in a darkened or dimmed operating room environment. High-grade tumor cells containing PPIX absorb the excitation light and emit fluorescence (i.e., red fluorescence) having specific optical characteristics. The fluorescence may be observed by the healthcare professional from the surgical microscope.

Once the target tissue has been identified, the healthcare professional switches the surgical microscope back to standard white light illumination and continues to resect the target tissue. The healthcare professional switches back and forth between illuminating the tissue with white light and the excitation light throughout the surgical procedure to ensure the appropriate target tissue is being resected until the tumor resection is complete. Each time the target area is illuminated with the excitation light from the surgical microscope, the PPIX present at the tumor site may degrade due to photo-bleaching from being illuminated by the strong excitation light.

Fluorescence guided surgery increases the chances of GTR in high-grade tumors such as with glioblastoma tumors. At present, GTR of lower grade tumors is comparatively low because 5-ALA cannot be used to improve the outcome of lower-grade tumor resection as the tumor cells only emit a low level of fluorescence and the human eye is not sensitive enough to detect such low levels of fluorescence even with the use of the surgical microscope. A need exists for an improved system for fluorescence guided surgery that improves the chances of achieving GTR.

The background description provided here is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

### SUMMARY

According to a first aspect, a surgical method of assessing tumor margins of obscured tissue of a patient who was administered a fluorescing agent is provided. The method includes creating an access hole through a tissue of the patient and extending to a surgical site of the patient, accessing the surgical site through the access hole and resecting tissue at the surgical site of the patient with a surgical instrument to create a resection cavity with at least a portion of thereof being outside of the field of view of the surgical microscope and including the obscured tissue, positioning the surgical instrument in contact with the obscured tissue, collecting fluorescent emission from the obscured tissue with the surgical instrument, and controlling an indicator of a tissue detection system based on the fluorescent emissions from the obscured tissue as collected by the surgical instrument.

According to a second aspect, a surgical method of assessing tumor margins of obscured tissue of a patient who was administered a fluorescing agent is provided. The method includes providing a tissue detection system and a surgical microscope which has a field of view. The tissue detection system includes an optical system and a surgical instrument for resecting tissue. The surgical instrument includes a handle portion, a shaft extending from the handle portion, and at least one optical fiber extending to a distal end of the surgical instrument for transmitting excitation light and for receiving fluorescent emissions from the obscured tissue. The method further includes creating an access hole having a first width through a tissue of the patient and extending to a surgical site of the patient, accessing the surgical site through the access hole and resecting tissue at the surgical site of the patient with the surgical instrument to create a resection cavity having a second width which is larger than the first width and at least a portion of the resection cavity being outside of the field of view of the surgical microscope and including the obscured tissue, positioning the surgical instrument through the access hole and within the resection cavity such that the surgical instrument is in contact with the obscured tissue, collecting fluorescent emission from the obscured tissue using the at least one optical fiber of the surgical instrument, and controlling an indicator of the tissue detection system based on the fluorescent emissions from the obscured tissue as collected by the at least one optical fiber of the surgical instrument.

According to a third aspect, a surgical method of assessing a skull-base tumor of a patient who was administered a fluorescing agent is provided. The method includes providing a tissue detection system including an optical system and a surgical instrument. The surgical instrument includes at least one optical fiber extending to a distal end of the surgical instrument for transmitting excitation light and for receiving fluorescent emissions from the skull-base tumor. The method further includes creating a hole through a proximal and distal wall of a sphenoid bone of a skull of the patient, inserting the surgical instrument trans-nasally through the proximal and distal walls of the sphenoid bone such that the distal end of the optical fiber is positioned in contact with the skull-base tumor of the patient, and controlling an indicator of the tissue detection system based on the fluorescent emissions from the skull-base tumor.

Any of the above aspects can be combined in part or in whole with any other aspect. Any of the above aspects, whether combined in part or in whole, can be further combined with any of the following implementations, in full or in part.

In some implementations, the resection cavity may include a first resection volume and a second resection volume. The second resection volume may be outside of the field of view of the surgical microscope and include the obscured tissue. As such. positioning the surgical instrument in contact with the obscured tissue may include positioning the surgical instrument at least partially outside of the field of view of the surgical microscope and in contact with a portion of the second resection volume.

In some implementations, the surgical instrument may include a least one bend in the shaft of the surgical instrument. Further, the surgical instrument may be an ultrasonic resection tool or bipolar forceps.

In some implementations, the surgical instrument may be a surgical suction tool configured to couple to a vacuum source and apply a suction force to the surgical site. The suction tool may include an aperture for controlling the suction force (i) manually and/or (ii) in response to the indicator of the tissue detection system. The surgical suction tool may be operable in multiple modes including an observation mode and a resection mode. In the observation mode, the surgical suction tool may be configured to detect fluorescent emission from the obscured tissue without applying the suction force. In the resection mode, the surgical suction tool may be configured to detect fluorescent emission from the obscured tissue while applying the suction force. The method may include maintaining the surgical suction tool in the observation mode and switching the surgical suction tool from the observation mode to the resection mode based on the fluorescent emission from the obscured tissue as collected by the optical fiber of the surgical suction tool.

In some implementations, a resection function of the surgical instrument may be controlled based on detected fluorescence during an operation. As such, the method may include positioning the surgical instrument in contact with a first portion of the obscured tissue, detecting fluorescent emission from the first portion of the obscured tissue and resecting the first portion of the obscured tissue with the surgical instrument in response to the detection, determining that the optical fiber of the surgical instrument does not detect fluorescent emissions and disabling a resection function of the surgical instrument in response to the determination, moving the surgical instrument within the resection cavity to a position which is in contact with a second portion of the obscured tissue, detecting fluorescent emission from the second portion of the obscured tissue, enabling the resection function of the surgical instrument in response to the detection, and resecting the second portion of the obscured tissue with the surgical instrument.

In some implementations, the surgical instrument may need to be repositioned within the resection cavity more than the tissue would permit without being displaced by the instrument. In these instances, the method may further include resecting a first portion of the obscured tissue with the surgical instrument, determining that the optical fiber of the surgical instrument no longer detects fluorescent emissions, displacing tissue without resecting the tissue, positioning the distal end of the surgical instrument in contact with a second portion of the obscured tissue while displacing tissue within the resection cavity, and resecting the first portion of the obscured tissue.

In some implementations, the indicator may be coupled to the surgical instrument. Additionally, the tissue detection system may include a controller in communication with the optical system and the indicator, and the indicator may include an optical fiber connected to a light source. The light source may be controlled by the controller based on the fluorescent emissions from the obscured tissue as collected by the at least one optical fiber of the surgical instrument, or based on the fluorescent emissions from the skull-base tumor. The indicator may be configured to illuminate the resection cavity with a light color which is different from the blue light emitted from the at least one optical fiber. The indicator may include a reposition indicator configured to indicate to a user that the surgical microscope should be repositioned to change the field of view of the surgical microscope to cause at least a portion of the obscured tissue to be within the field of view. The indicator may be visible outside of the skull of the patient.

In some implementations, positioning the surgical instrument through the access hole and within the resection cavity may obscure at least a portion of the field of view of the surgical microscope and the obscured portion of the field of view may include shadowed tissue. Thus, the method may further include positioning the surgical instrument such that the surgical instrument is in contact with the shadowed tissue, collecting fluorescent emission from the shadowed tissue using the at least one optical fiber of the surgical instrument, and controlling the indicator of the tissue detection system based on the fluorescent emissions from the shadowed tissue as collected by the at least one optical fiber of the surgical instrument.

In some implementations, the skull-base tumor may be a pituitary tumor.

In some implementations, an endoscope is utilized to detect fluorescence emitted from the skull-base tumor. As such, the method may include inserting an endoscope trans-nasally through the proximal and distal wall of the sphenoid bone such that a distal end of the endoscope is positioned adjacent the skull-base tumor of the patient. The surgical instrument may be inserted through a first nostril of the patient and the endoscope is inserted through a second nostril of the patient. Further, the endoscope may be coupled to a display which includes the indicator and/or a graphical user interface. The graphical user interface may include the indicator.

### BRIEF DESCRIPTION OF THE DRAWINGS

Advantages of the present disclosure will be readily appreciated as the same becomes better understood by reference to the following detailed description when considered in connection with the accompanying drawings.

FIG. 1 depicts a chart depicting the visibility of red fluorescence of tumor tissue when viewed from a surgical microscope according to the teachings of the prior art.

FIG. 2 depicts a neurosurgical system according to the teaching of the present disclosure.

FIG. 3 depicts a functional block diagram of a neurosurgical system according to the teachings of the present disclosure.

FIG. 4 depicts an example suction tool of the neurosurgical system according to the teachings of the present disclosure.

FIGS. 5A and 5B depict an example handle of a suction tool of the neurosurgical system according to the teachings of the present disclosure.

FIG. 6 depicts an ultrasonic surgical system of a neurosurgical system according to the teachings of the present disclosure.

FIG. 7 depicts a tissue detection system of a neurosurgical system according to the teachings of the present disclosure.

FIG. 8 depicts a functional block diagram of a tissue detection system of a neurosurgical system according to the teachings of the present disclosure.

FIGS. 9A and 9B depict an optical system of a tissue detection system according to the teachings of the present disclosure.

FIGS. 10A and 10B depict an exploded view of some components of the optical system of a tissue detection system according to the teachings of the present disclosure.

FIG. 11 depicts a view of an interior portion of a control console of a tissue detection system according to the teachings of the present disclosure.

FIGS. 12A-E depict a sample element of a tissue detection system according to the teachings of the present disclosure.

FIG. 13 depicts an excited spectral signal generated by a spectrometer of a tissue detection system according to the teachings of the present disclosure.

FIGS. 14A and 14B depict a first modified spectral signal and a second modified signal generated by a controller of a tissue detection system according to the teachings of the present disclosure.

FIG. 15 depicts gaussian curves fit to a second modified signal generated by a controller of a tissue detection system according to the teachings of the present disclosure.

FIG. 16 depicts Protoporphyrin IX (PPIX) intensity generated by a controller of a tissue detection system according to the teachings of the present disclosure.

FIG. 17 depicts a flowchart for a surgical resection procedure performed with a neurosurgical system according to the teachings of the present disclosure.

FIGS. 18A and 18B depict a sample element of a tissue detection system coupled to an ultrasonic handpiece assembly according to the teachings of the present disclosure.

FIGS. 19A and 19B depict a sample element and an indicator element of a tissue detection coupled to bipolar forceps of a surgical system according to the teachings of the present disclosure.

FIG. 20 depicts a sample element coupled to a suction tool of a suction system with a jacket removed according to the teachings of the present disclosure.

FIG. 21 depicts a sample element coupled to a suction tool of a suction system according to the teachings of the present disclosure.

FIG. 22 depicts a sample element coupled to a suction tool of a suction system according to the teachings of the present disclosure.

FIG. 23 depicts a sample element coupled to a suction tool of a suction system according to the teachings of the present disclosure.

FIG. 24 depicts a sample element coupled to a suction tool including a jacket according to the teachings of the present invention.

FIG. 24 depicts one implementation of a sample element coupled to a suction tool including a jacket according to the teachings of the present disclosure.

FIG. 25 depicts a side perspective view of a portion of the suction tool of FIG. 24, shown with the jacket removed for exemplary purposes.

FIGS. 26A and 26B depict a perspective view of a portion of the suction tool of FIG. 24, shown with a handle of the suction tool including an opening.

FIG. 27A depicts a bottom perspective view of a portion of the distal end of the suction tool of FIGS. 24-26B, shown with the suction tool defining an outer channel extending to the distal end of the suction tool.

FIG. 27B depicts a top perspective view of a portion of a distal end of a suction tool according to another implementation.

FIG. 28 depicts an optics block according to the teachings of the present disclosure.

FIG. 29 depicts the optics block of FIG. 28, shown with the optics block coupled to a connector according to the teachings of the present disclosure.

FIG. 30 depicts a bottom perspective view of a portion of the optics block, shown with the optics block including an adjustment mechanism according to the teachings of the present disclosure.

FIG. 31 depicts an exemplary block diagram for differentiating brain tumor tissue from healthy brain tissue according to the teachings of the present disclosure.

FIG. 32 depicts another implementation of a sample element coupled to a suction tool including a sleeve according to the teachings of the present disclosure.

FIG. 33 depicts a perspective view of a portion of the suction tool of FIG. 32, shown with a handle of the suction tool including an opening.

FIG. 34 depicts a side view of a portion of the handle of the suction tool of FIG. 33.

FIG. 35 depicts a cross-sectional perspective view of a portion of the distal end of the suction tool of FIGS. 32-34, shown with the suction tool defining an outer channel extending to the distal end of the suction tool.

FIG. 36A depicts a close-up perspective view of a portion of the distal end of the suction tool of FIGS. 32-34, shown with a distal sleeve removed from the distal end.

FIG. 36B depicts a close-up perspective view of a portion of the distal end of another implementation of the suction tool, shown with a distal sleeve removed from the distal end.

FIG. 37 depicts a perspective view of the distal end of the suction tool of FIG. 36A, shown with the distal sleeve and a proximal sleeve removed from the distal end.

FIG. 38 depicts another cross-sectional perspective view of a portion of the suction tool of FIG. 35, showing the handle of the suction tool.

FIG. 39 is a cross-sectional side view of the suction tool and handle of FIG. 32.

FIG. 40 is a cross-sectional top view of the handle of the suction tool of FIG. 32.

FIG. 41 is a cross-sectional view of the distal end of the suction tool of FIGS. 32-34, shown with the distal sleeve removed from the distal end.

FIG. 42 is a schematic side view of a manufacturing process of the suction tool of FIG. 32, showing a suction tube, an optical fiber, and the proximal jacket in an unrecovered state.

FIG. 43 is a schematic end view of the manufacturing process of the suction tool of FIG. 32, showing the suction tube, the optical fiber, and the proximal jacket in a partially recovered state.

FIG. 44 is an exemplary block diagram of the manufacturing process of FIGS. 42 and 43.

FIG. 45 is a side view of an optical connector of the suction tool of FIG. 32 with a portion of the casing removed.

FIG. 46 includes a flow diagram depicting a method of assessing tumor margins of obscured tissue.

FIGS. 47A-47D are illustrations of various steps of the method shown in FIG. 46.

FIG. 48 includes a flow diagram depicting a method of assessing a skull-base tumor.

FIGS. 49A-49C are illustrations of various steps of the method shown in FIG. 48.

In the drawings, reference numbers may be reused to identify similar and/or identical elements.

### DETAILED DESCRIPTION

The present inventors realized that there exists a need for a neurosurgical tumor resection system and/or method that is capable of detecting low levels of fluorescence in white light operating conditions (i.e., not requiring a darkened or dimmed operating room) while in the process of resecting the tumor. There also exists a need for a system that can reduce the amount of time that the target area is illuminated with excitation light to reduce the effects of photo-bleaching. Additionally, there exists a need for a system that can illuminate excitation light in deep cavities as surgical microscope fail to adequately illuminate excitation light in deep cavities. Lastly, there exists a need for a system that assists in intraoperative detection of the anaplastic focus of the tumor which is of importance because finding the anaplastic focus is imperative for precise histopathological diagnosis and optimal patient treatment.

While the disclosure specifically discusses a surgical procedure related to resection of target tissue of a brain tumor with the administration of 5-ALA to visualize fluorescence of Protoporphyrin IX (PPIX), the teachings of the present disclosure may be extended to other types of surgical procedures, to detect other types of tissue, and to detect other types of fluorophores (Hypericin, Hexvix, Idocyanine Green “ICG”, etc.). For example, ICG may be administered to help a healthcare professional visualize blood vessels during the surgical procedure. ICG may bond to plasma protein found in blood. ICG is excited by near infrared light and emits near infrared light having a slightly longer wavelength than the near infrared light that excited the ICG.

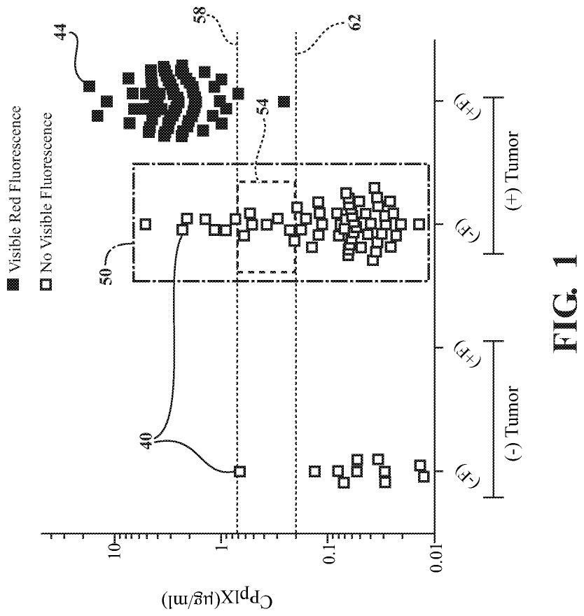

With respect to FIG. 1, chart 10 depicts the visibility of the red fluorescence of PPIX when viewed from a surgical microscope. During brain tumor resection surgery, target tissue (i.e., tumor tissue) including elevated concentrations of PPIX may inadvertently be missed when tumor resection surgery is performed according to the systems of the prior art (i.e., with a surgical microscope) which leads to less than GTR. Thus, a more accurate way of detecting elevated concentrations of PPIX would prove to be very beneficial in helping to achieve GTR. Hollow squares 40 indicate specimens that produce no visible fluorescence and solid squares 44 indicate specimens that produce visible fluorescence. The y-axis shows the accumulated levels of PPIX concentration (CPpIX) above a threshold of 0.01 μg/mL. The x-axis indicates visible fluorescence (+F) and non-visible fluorescence (−F) for healthy tissue and for target tissue. Region 50 represents a false negative region in which PPIX was present in the specimen but did not produce visible light. In particular, a sub-region 54 within the region 50 includes specimens below a level 58 that when viewed from a surgical microscope but still considered to include elevated levels 62 of CPpIX. Thus, if the healthcare professional were to miss PPIX corresponding to the sub-region, GTR would not be achieved.

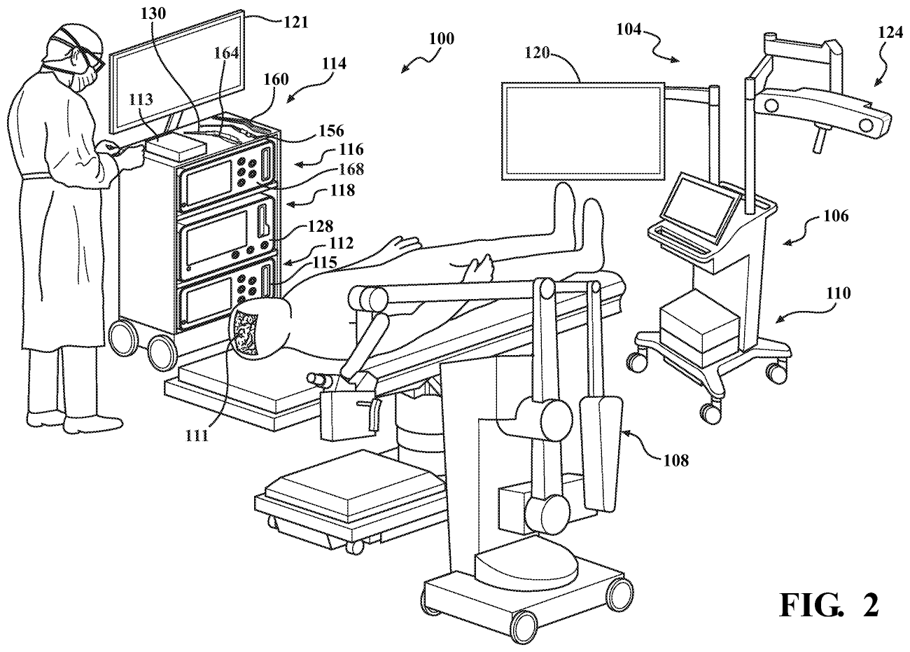

With reference to FIG. 2, the neurosurgical system 100 is provided that solves the shortcomings of the prior art. The neurosurgical system 100 may include a surgical navigation system 104, a surgical microscope 108, a surgical cart 114, and a suction system 113. The surgical navigation system 104 includes a cart assembly 106 that houses a navigation computer 110. The navigation computer 110 may also be referred to as the navigation controller. A navigation interface is in operative communication with the navigation computer 110. The navigation interface may include one or more input devices may be used to input information into the navigation computer 110 or otherwise to select/control certain aspects of the navigation computer 110. The navigation interface includes one or more displays 120. Such input devices may include interactive touchscreen displays/menus, a keyboard, a mouse, a microphone (voice-activation), gesture control devices, or the like.

The navigation computer 110 may be configured to store one or more pre-operative or intra-operative images of the brain. Any suitable imaging device may be used to provide the pre-operative or intra-operative images of the brain. For example, any 2D, 3D or 4D imaging device, such as isocentric fluoroscopy, bi-plane fluoroscopy, ultrasound, computed tomography (CT), multi-slice computed tomography (MSCT), magnetic resonance imaging (MRI), positron emission tomography (PET), optical coherence tomography (OCT). The images may also be obtained and displayed in two, three or four dimensions. In more advanced forms, four-dimensional surface rendering regions of the body may also be achieved by incorporating patient data or other data from an atlas or anatomical model map or from pre-operative image data captured by MRI, CT, or echocardiography modalities.

The navigation computer 110 may generate the one or more images of the brain on a display 120. The navigation computer 110 may also be connected with the surgical microscope 108. For example, the display 120 may show an image corresponding to the field of view of the surgical microscope 108. When the navigation computer 110 may include more than one display, with one such display showing the field of view of the surgical microscope 108 while the other such display may show a pre-operative or intra-operative image of the brain.

The tracking system 124 is coupled to the navigation computer 110 and is configured to sense the position of one or more tracking elements attached to a surgical tool or the patient. The tracking system 124 may be configured to track active or passive infrared tracking elements attached to the surgical tool or the patient. An example of a surgical navigation system 104 that may be used is Nav3i™ that is commercially available from Stryker. A surgical navigation system 104 may have various functions and features as described in U.S. Pat. Nos. 7,725,162 B2 and 11,369,438 B2 which are hereby incorporated by reference in their entireties.

The surgical microscope 108 includes one or more objectives configured to provide magnification in a range (e.g., from about 2 times to about 50 times). The surgical microscope 108 can have a field of view having an area of a predetermined range. The surgical microscope 108 is configured for fluorescence microscopy, for example, to detect PPIX. The surgical microscope 108 may include one or more excitation sources (e.g., an excitation source configured to emit light in the visible light spectrum, or an excitation source configured to emit light in the infrared spectrum) for illuminating the brain tissue 111 with excitation light to cause the PPIX to fluoresce. The surgical microscope 108 may also include a camera capable of detecting radiation at the fluorescent wavelengths of PPIX or ICG.

The surgical cart 114 may include a surgical system 112, a suction system 113, a tissue detection system 116, and an ultrasonic surgical system 118. A display 121 may be coupled to the surgical cart and operatively connected to the surgical system 112, the tissue detection system 116, and/or the ultrasonic surgical system 118 to display information related with each respective system 112, 116, and 118. A healthcare professional may use the ultrasonic surgical system 118 and/or the surgical system 112 to ablate target tissue of the brain of the patient. The ultrasonic surgical system 118 may include an ultrasonic control console 128 and an ultrasonic handpiece assembly 130.

The suction system 113 may include a suction tool 156 and suction unit 117 to control various aspects of the suction tool 156. A suction tube may connect the suction tool 156 to the suction system 113. The suction system 113 may receive suction from a vacuum source, such as a vacuum outlet of a medical facility. The suction system 113 may include one or more regulators or one or more adjustment valves for controlling the suction pressure received from the vacuum source. The suction system 113 may also include one or more containers for storing the waste collected by the suction tool 156. In an example, the suction system 113 may correspond to a wall suction unit. In another example, the suction system 113 may correspond to a portable suction unit. The suction system 113 and the suction tool 156 may have various features, as described in U.S. Pat. No. 9,066,658 B2 and U.S. Pat. Pub. No. 2018/0344993 A1 which are hereby incorporated herein by reference in its entirety.

The surgical system 112 may include a surgical tool, such as bipolar forceps 160, and a surgical control console 115 to control various aspects of the surgical tool. The healthcare professional may also use the surgical tool to perform any surgical operation on the tissue. For example, to ablate the tissue or to cauterize the tissue. The bipolar forceps may have features, as described in U.S. Pat. No. 8,361,070 B2 which is hereby incorporated by reference in its entirety. While the disclosure discusses and illustrates that the surgical tool may include bipolar forceps 160, the surgical system 112 and surgical tool may include other tools, such as a neuro stimulator, a dissector, or an ablation device (e.g., an RF ablation device and/or a laser ablation device). For example, the surgical system and/or surgical tools may have various features as described in U.S. Pat. No. 8,267,934 B2, which is hereby incorporated by reference in its entirety. Any number of surgical systems and any number of surgical tools may be employed by the healthcare professional in performing the surgical procedure.

The tissue detection system 116 may include a control console 168 and a sample element 164 (illustrated as coupled to the ultrasonic handpiece assembly 130). The control console 168 may provide the healthcare professional with a real-time indication via the sample element 164 when brain tissue 111 corresponds to the target tissue. The sample element 164 may also be coupled to the bipolar forceps 160, the suction tool 156, or other surgical tools as will be described in greater detail below. The tissue detection system 116 determines when the brain tissue 111 corresponds to target tissue based on fluorescence emitted by the target tissue caused by the fluorophore. In an example, the fluorophore may correspond to PPIX. In another example, the fluorophore may correspond to ICG. As will be discussed in greater detail below, based on the intensity and the wavelengths of the fluorescence emitted by PPIX, the tissue detection system 116 may determine that the target tissue is present.

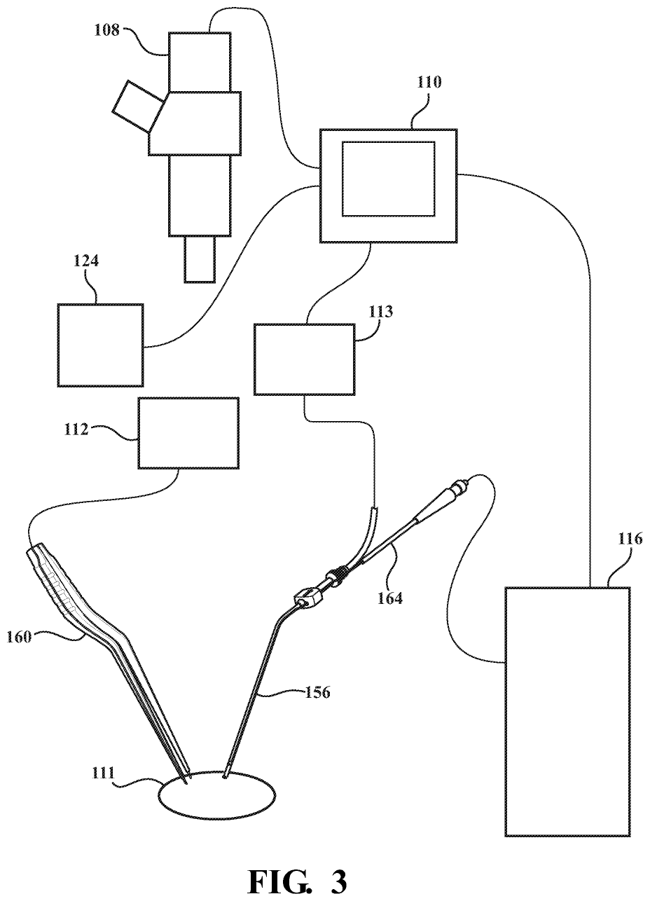

With reference to FIG. 3, a schematic of the neurosurgical system 100 is shown. The tissue detection system 116, although capable of performing a similar function (i.e., allowing the healthcare professional to detect the presence of PPIX) to the surgical microscope 108, may be used in conjunction with the surgical microscope 108 to improve the outcome of a tumor resection procedure and the chances of achieving GTR.

During the surgical procedure, the healthcare professional may initially view the brain tissue 111 of the patient with the surgical microscope 108 under excitation light (e.g., the blue light) to identify which portion of the brain tissue 111 corresponds to the target tissue evidenced by the red fluorescence. The healthcare professional may switch the surgical microscope 108 back to standard white light illumination for better visibility and begin resection of the target tissue. Since the sample element 164 is coupled to the suction tool 156, the healthcare professional does not have to account for any additional surgical tools (i.e., optical probes or the like) in the sterile field. The healthcare professional may perform the resection of the target tissue with the bipolar forceps 160 in the one hand and the suction tool 156 in the other hand.

As the healthcare professional is resecting the target tissue, the control console 168 may function to provide the healthcare professional with a real-time indication of the target tissue in the brain tissue 111 by activation of an indicator (discussed in greater detail below) of the sample element 164. The tissue detection system 116 according to the teachings of the present disclosure prevents the healthcare professional from having to switch back and forth between the various illumination settings of the surgical microscope 108 (i.e., illuminating the tissue with excitation light and white light) as the healthcare professional is performing resection of the target tissue. This becomes especially important as the healthcare professional approaches the margin of the target tissue because it is desirable for the healthcare professional to achieve GTR but to leave as much healthy tissue intact as possible.

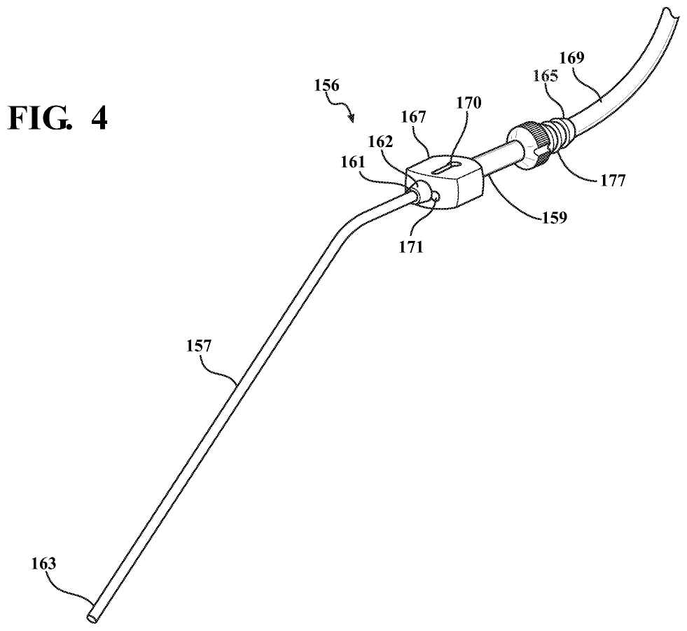

With reference to FIG. 4, the suction tool includes a suction cannula 157 and a handle 159. The suction cannula 157 defines a lumen for suctioning fluid, debris, and tissue from a patient. The handle 159 is tubular shaped with a control portion 167 that may be square shaped. A distal end 162 may be tapered and is configured to receive a proximal end 161 of a suction cannula 157. A proximal end 165 of the handle 159 includes a vacuum fitting which may be configured to receive a suction tube 169 which is connected to the vacuum source which generates the suction pressure. The vacuum fitting may be a standard barbed fitting, quick disconnect, or any other suitable fitting known in the art to allow the suction tube to be fluidly coupled to a vacuum source.

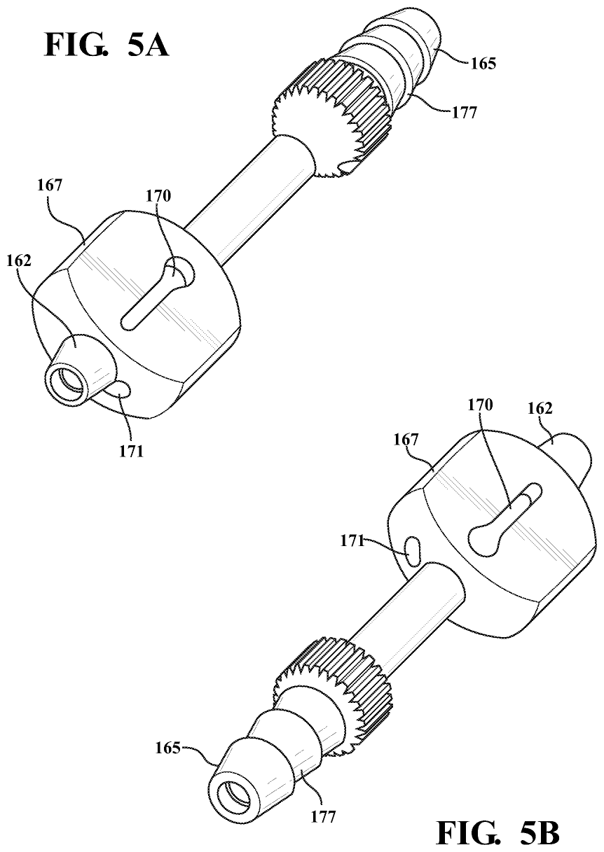

With additional reference to FIGS. 5A and 5B, the control portion 167 may include a teardrop shaped control 170 for regulation of suction pressure. For example, when no portion of the teardrop shaped control 170 is covered by the healthcare professional, suction pressure may be minimal, and when the teardrop shaped control 170 is covered completely, suction pressure may be at its maximum. While the control portion 167 is described as including a teardrop shaped control, the control portion 167 may include another suitable input such as a button or different shaped control to allow the healthcare professional to vary the suction pressure. The control portion includes a through bore 171 for receiving the sample element 164, as will be discussed in greater detail below. The healthcare professional holds the suction tool 156 from its handle 159, manipulating the suction tool 156 so that the distal end 163 contacts the tissue of the patient during the surgical procedure in order to provide suction at the desired location. While the suction tool 156 is described as having a Fukushima configuration, other configurations are contemplated such as a Frazier or Poole configuration.

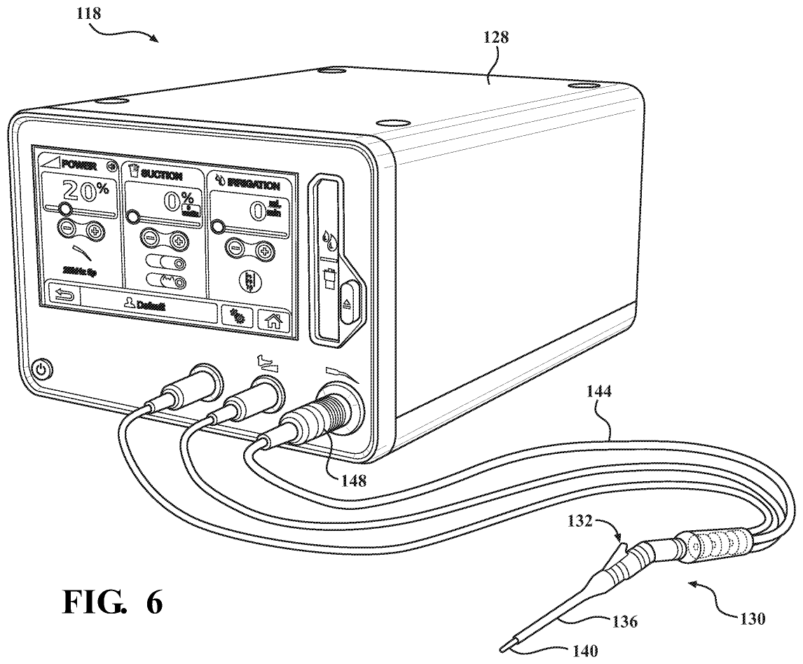

With reference to FIG. 6, the ultrasonic handpiece assembly 130 may comprise an ultrasonic handpiece 132 comprising a proximal end and distal end. The ultrasonic handpiece assembly 130 may further comprise sleeve 136 and an ultrasonic tip 140 that may be coupled to the distal end of the ultrasonic handpiece 132. The sleeve 136 may be configured to provide irrigation to the ultrasonic tip 140 and/or the surgical site. It is further contemplated that the sleeve 136 may also be configured to provide aspiration to the ultrasonic tip 140. The ultrasonic tip 140 may comprise a cutting feature that is configured to ablate, cut, shape, and/or remove biological tissue. The ultrasonic handpiece assembly 130 may have various features, as described in U.S. Pat. Nos. 6,497,715 B2; 6,955,680 B2; and 6,984,220 B2 and PCT Publication WO 2020/068756 A1; which are hereby incorporated herein by reference in their entirety.

The ultrasonic handpiece assembly 130 may also comprise a cable 144 or other power cord comprising a power connector 148 or adapter configured to couple the ultrasonic handpiece assembly 130 to a power supply, such as the ultrasonic control console 128 configured to regulate the various aspects of the ultrasonic handpiece assembly 130. The ultrasonic control console 128 may also be configured to provide irrigation and/or aspiration via one or more tubes (not shown) connected to the handpiece assembly 130 and regulate the irrigation and/or aspiration functions of the ultrasonic handpiece assembly 130 to optimize performance of the ultrasonic handpiece assembly 130. An example of ultrasonic surgical systems that may be used are commercially available from Stryker including Sonopet IQ Ultrasonic Aspirator. The ultrasonic control console 128 may control various operation parameters based on signals received from the tissue detection system 116.

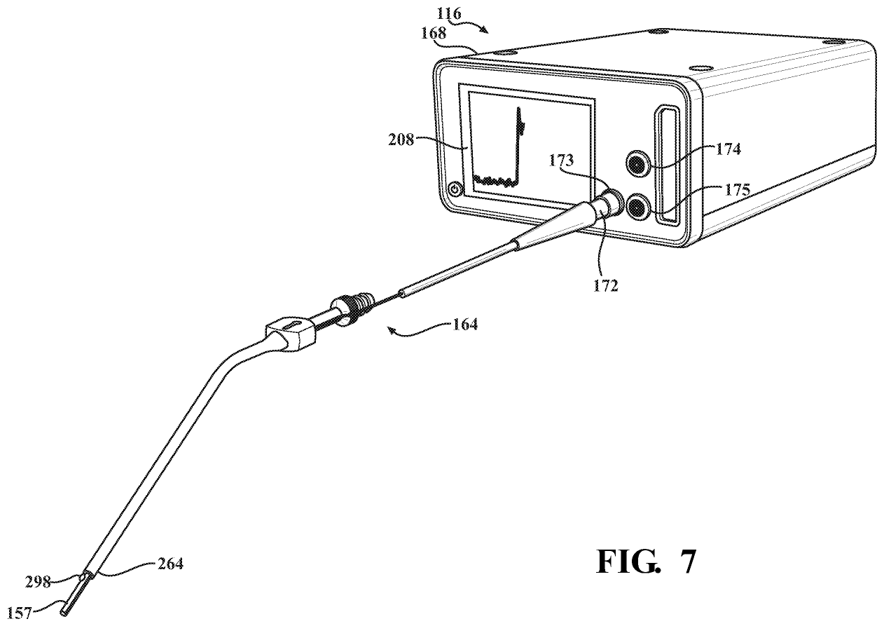

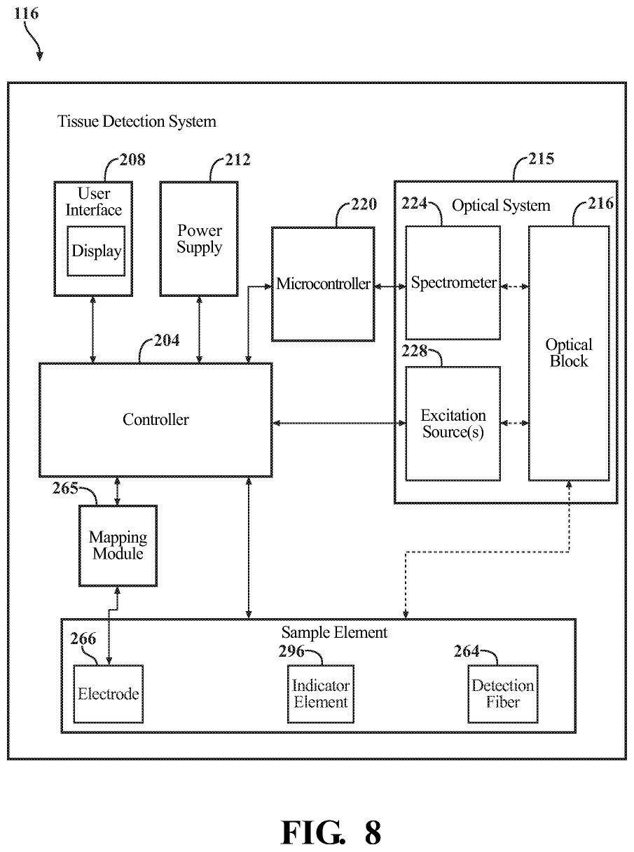

With reference to FIGS. 7 and 8, the tissue detection system 116 includes a sample element 164 and a control console 168. The sample element 164 is connected to the control console 168 via a connector 172. The sample element 164 may include a detection fiber 264, an indicator element 296, and an electrode 266 as discussed in greater detail below. The control console 168 may include a controller 204, a user interface 208, a power supply 212, an optical system 215, a microcontroller 220, and a mapping module 265. The optical system 215 may include an optics block 216, a spectrometer 224, an excitation source 228, and an optical connector 229. The function of each component will be discussed in greater detail below.

The user interface 208 may include a display for displaying output from the controller 204. The user interface 208 may also include one or more inputs (e.g., a push button, a touch button, a switch, etc.) configured for engagement by the healthcare professional. The power supply 212 may supply power to various components of the control console 168. The control console 168 may include a probe port 173 in which the connector 172 of the sample element 164 is connected. The detection fiber 264 may then be connected to the optics block 216 via the optical connector 229. The control console 168 may also include an electrical port 174 for establishing communication link to the surgical system 112 and the ultrasonic surgical system 118. The control console 168 may also include an indicator port 175 for connection to an indicator element 164, as will be discussed in greater detail below.

The mapping module 265 may include a device configured to generate stimulation signals for an electrode 266 that is configured to deliver the stimulation signals to the brain tissue 111. During surgical resection of the target tissue, the healthcare professional may need to map the brain tissue 111 in order to determine which areas of the brain tissue 111 correspond to functionally important areas. For example, functionally important areas of the brain that are responsible for speech or motor skills may be chosen to be avoided even when it is determined that these areas include target tissue if the target tissue cannot be removed without impacting the underlying function of the area. The device may be configured to generate an electric current which is then applied to the brain tissue 111 by the healthcare professional via an electrode. The electrode may be a standalone electrode disposed or coupled to an outer surface of the sample element 164 or the electrode may be integrated within the sample element 164, as discussed in greater detail below. The mapping module 265 and/or electrode 266 may have various functions and features as described in US Pat. Pub No. 2022/0400972 A1 and U.S. Pat. No. 7,150,737 B2 which are hereby incorporated by reference in their entirety. The controller 204 may be configured to generate an alert based on the results of the stimulation of the electrode 266 on the brain tissue 111. For example, the controller 204 may generate an alert to be displayed on the user interface 208, the display 120, or the display 121. The alert may indicate to the healthcare professional whether or not the brain tissue 111 corresponds to a functionally important area such as an area associated with motor function or speech function.

The excitation source 228 may illuminate the target tissue with excitation light via the detection fiber 264. The excitation source 228 may be configured to emit the excitation light (e.g., blue light at about 405 nm or blue light in the range of 400 nm to 500 nm). The excitation source 228 may also be configured to emit excitation light corresponding to other wavelengths such as wavelengths associated with the rest of the visible light spectrum other than blue light (e.g., greater than 500 nm but less than 700 nm), wavelengths associated with ultraviolet light spectrum (less than 400 nm) and/or infrared light spectrum (greater than 700 nm). The excitation source 228 may include any number of light sources such as a light emitting diode (LED), a pulsed laser, a continuous wave laser, a modulated laser, a filtered white light source, etc. In implementations where the excitation source is an LED, the device can be used without concern regarding eyes of people in the operating room.

In certain instances, the excitation source may be further configured to emit excitation light corresponding to different wavelengths than described above. In this implementation, the excitation sources may be referred to as a first excitation source 228 and a second excitation source, with the first excitation source 228 being configured to emit a first excitation light at the predetermined wavelength of the visible light spectrum and the second excitation source configured to emit infrared light at a second wavelength range corresponding to the infrared light spectrum (e.g., 700 nm to 1 mm). When two excitation sources are present, the first excitation source 228 may be configured to emit light which would excite a first fluorophore such as PPIX, while the second excitation source is configured to emit light which would excite a second fluorophore such as ICG.

The controller 204 may control operation of the excitation source 228. The controller 204 may control operation of the excitation source 228 by varying operating parameters of the excitation source 228. The operating parameters may correspond to a time setting, a power setting, or another suitable setting. The time setting may include a pulse width. The pulse width may be based on the integration time of the spectrometer 224. The integration time of the spectrometer 224 is discussed in greater detail below.

The detection fiber 264 may be coupled to the optical connector 229. When the sample element 164 is coupled to the surgical tool (i.e., the ultrasonic handpiece assembly 130, the suction tool 156, or the bipolar forceps 160) the distal end 272 of the detection fiber 264 is adjacent to the working portion of the surgical tool and allows for the excitation light to be delivered to the target tissue.

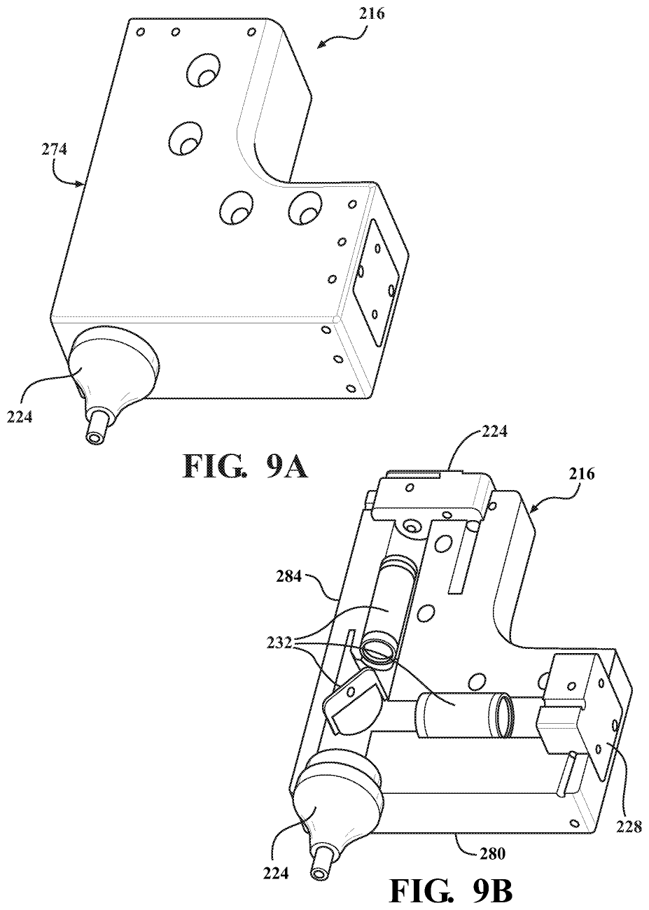

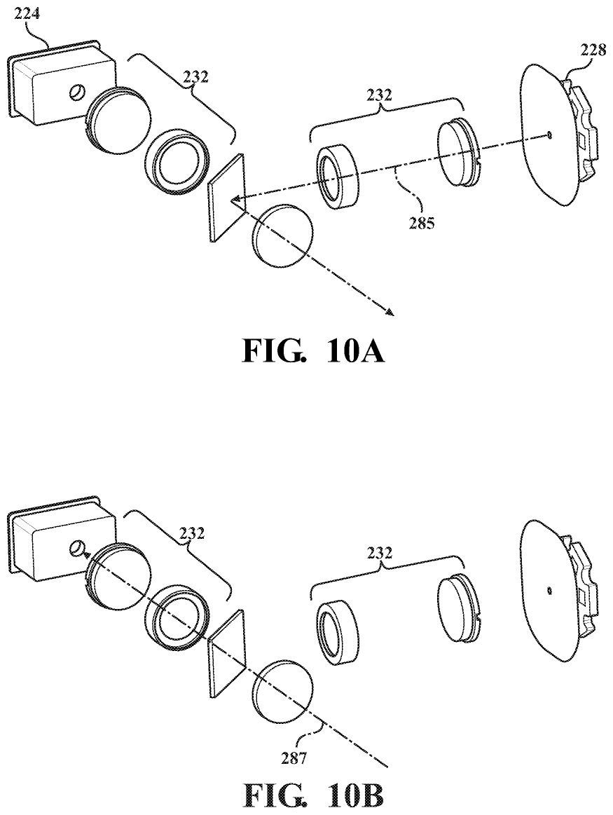

With reference to FIGS. 9A and 9B, the optics block 216 is shown. The optical connector 229 may be coupled to the optics block 216. The optics block 216 may include an outer casing 274 constructed of metal or another suitable material and may fully enclose components 232 of the optics block 216. FIG. 9B shows the optics block 216 with the top of the casing removed such that the components 232 of the optics block 216 are visible. The optics block 216 may be L-shaped and include a first portion 280 and a second portion 284. The excitation source 228 may be coupled to the first portion 280 of the optics block 216. The spectrometer 224 may be coupled to the second portion 284 of the optics block 216.

With additional reference to FIGS. 10A and 10B, an exploded view of the components 232 of the optical system 215 is shown illustrating an optical path 285 for the excitation light and the optical path 287 for light collected from the brain tissue 111. The first portion 280 may include the optical path 285 for the excitation light to travel from the one or more excitation sources 228 to the brain tissue 111 via the detection fiber 264. The optical path 285 may be defined by the components 232 in the first portion 280 of the optical block. The second portion 284 may include the optical path 287 for the collected light to travel from the brain tissue 111 via the detection fiber 264 to the spectrometer 224. The optical path 287 may be defined by the components 232 in the second portion 284 of the optical block. The components 232 of the optical block may optical components such as one or more laser line filters and one or more long-pass filters. The optics block 216 may include other optical components such as one or more mirrors, lenses, optical connectors, optical fiber, and/or any other suitable optical components.

In FIG. 10A, the excitation source 228 emits the excitation light which travels through one or more components 232, such as a laser line filter and/or long pass filter. The laser line filter or bandpass filter may be configured to reject unwanted noise (e.g., lower level transitions, plasma, and glows) generated by the excitation source 228. Stated differently, the laser line filter may be configured to clean up the excitation light or make the excitation light more monochromatic. The long-pass filter may be configured to reflect the light down the detection fiber 264 and to the brain tissue 111. The excitation source 228 may be configured to deliver unfiltered excitation light (i.e., the filters may be omitted) via the detection fiber 264 to the target tissue. The detection fiber 264 may guide the excitation light to the brain tissue 111 via the sample element 164.

The detection fiber 264 may be configured to collect light (i.e., fluorescence and ambient light) from the brain tissue 111. The coupling of the sample element 164 to the surgical tool results in the distal end 272 being adjacent to the working portion of the surgical tool as to allow for the light to be collected from the target tissue.

Due to the presence of ambient light and/or background light caused by various sources in the operating room such as the surgical microscope 108, surgical lamps, or any other devices in the operating room, the light collected from the brain tissue 111 may include the ambient light and/or background light. Light collected by the detection fiber 264 passes through the components 232, such as the long pass filter, of the second portion 284 of the optics block 216. After the light passes through the components 232, the light may enter the spectrometer 224 which is coupled to the optics block 216.

Since lighting conditions may vary, light collection from the brain tissue 111 may be collected at a faster clock cycle, that is, greater than 50 kHz, in order to maintain a high signal-to-noise ratio with the spectrometer. For example, in some configurations, light collection may be collected at a rate of 1.8 MHz. In this way, this allows for a shorter light collection time and faster processing time to reduce effects of any type of noise and avoid saturation of the light collected, regardless of the lighting conditions.

The detection fiber 264 may be coupled to the optical connector 229. As discussed in greater detail below, the distal end 272 of the detection fiber 264 may include a lens or other transparent material such that when the sample element 164 is positioned on a surgical tool (i.e., the ultrasonic handpiece, the suction tool or the bipolar forceps) the coupling of the sample element 164 to the surgical tool results in the distal end 272 of the detection fiber 264 being adjacent to the working portion of the surgical tool as to allow for the excitation light to be delivered to the target tissue.

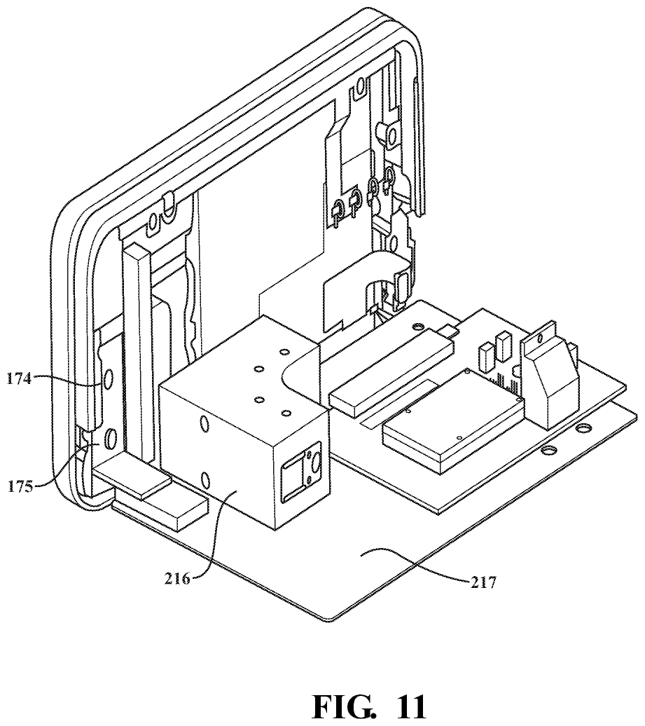

With reference to FIG. 11, a view of the control console 168 with the outer casing removed is shown. The optics block 216 may be fixed (e.g., via bolts) directly to a base 217 of the control console 168 to allow for heat dissipation for heat generated by one or more components of the optical system 215. The control console 168 may include enough void space such that more than one optics block 216 may be stacked inside the control console 168. For example, a second optics block with various optical components inside may be stacked on top of the optics block 216. The second excitation source may be coupled to the second optics block. The second optics block may include components that define an optical path for light generated by the second excitation source to reach the target tissue.

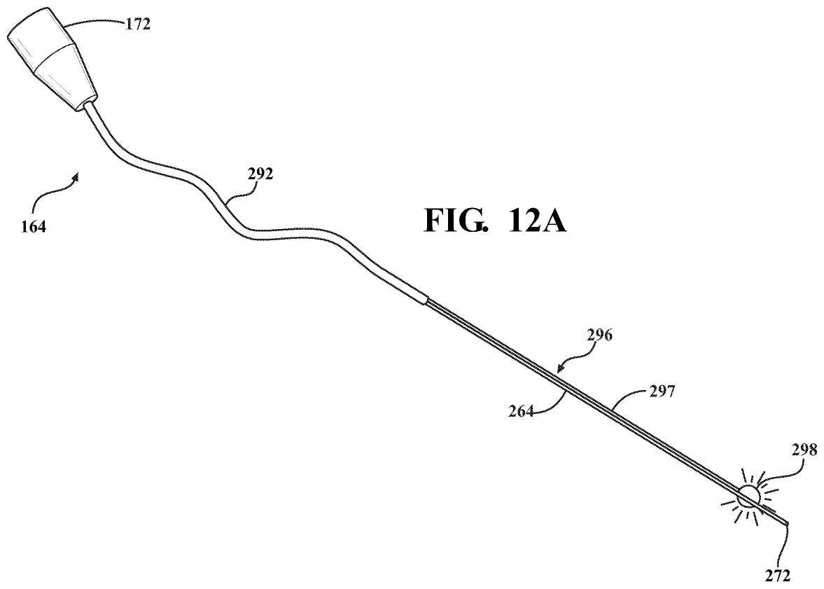

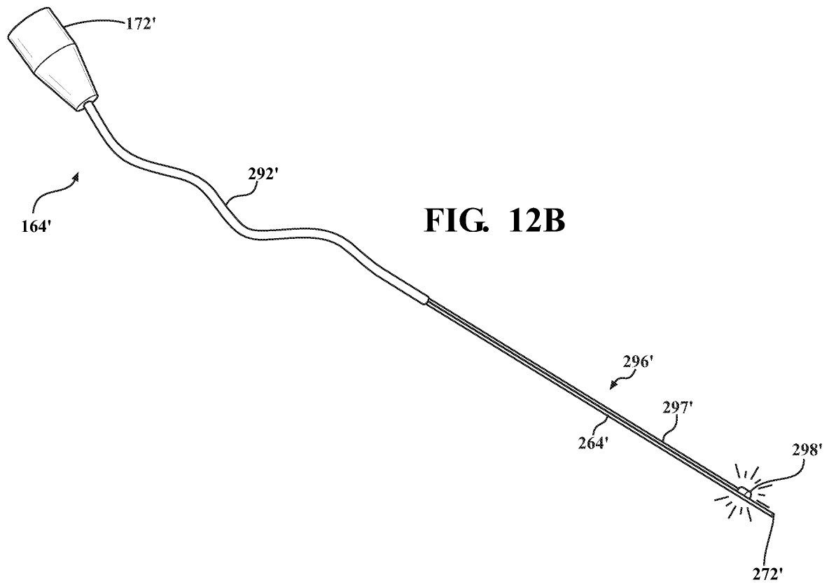

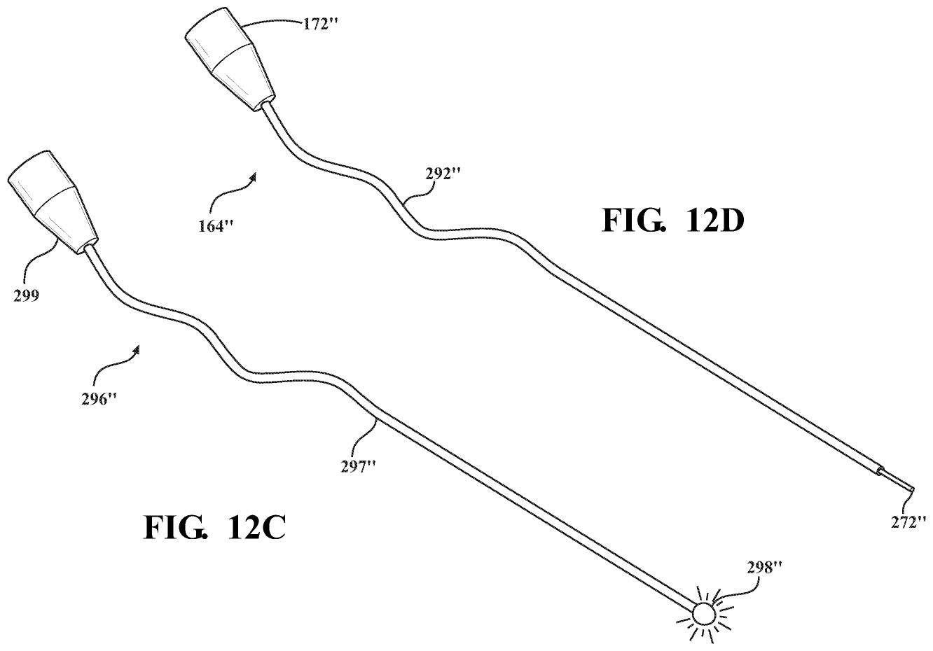

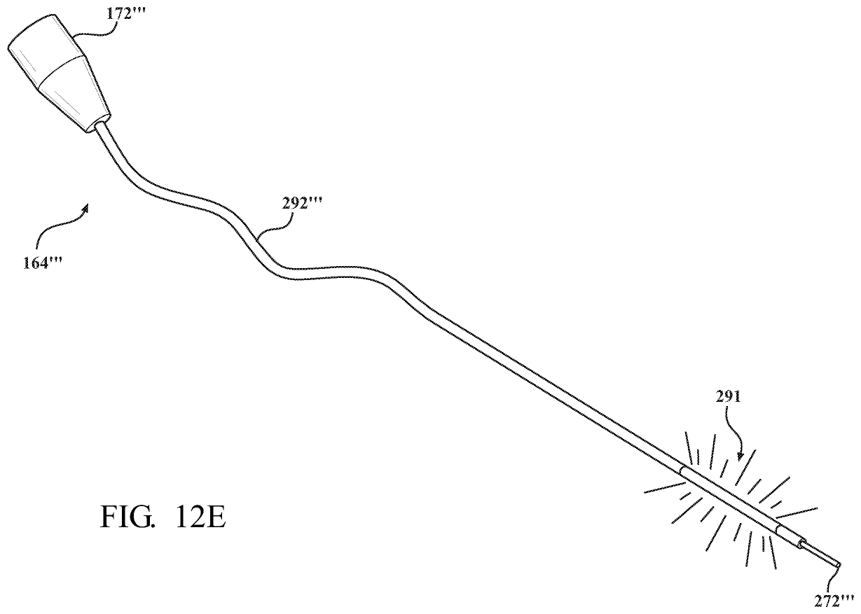

With reference to FIG. 12A, the sample element 164 shown. The sample element 164 may also include an indicator element 296. The indicator element 296 may be mounted to the sample element 164 or any surgical tool, including an optical probe. The indicator element 296 may include a transmission member 297 connected to an indicator 298. The indicator 298 may include one or more light emitting diodes or another suitable light source. The indicator 298 is configured to emit light in response to detection of tumorous or target tissue by the controller 204. The indicator 298 may be sphere shaped, dome shaped, cylinder shaped, or another suitable shape. A jacket 292 may enclose part of the detection fiber 264 and part of the indicator element 296, specifically the transmission member 297. Stated differently, the jacket 292 may terminate well in advance of the distal end 272 of the detection fiber 264 leaving the transmission element 297, the indicator 298 and the detection fiber 264 at least partially exposed. The jacket 292 may be made from any one of polyvinyl chloride, polyethylene, chlorinated polyethylene, and chlorosulfonated polyethylene/neoprene or another suitable material. The electrode 266 although not shown in FIG. 12A may be integrated with the sample element 164. For example, a distal end of the electrode may be positioned adjacent to a distal end of the detection fiber 264 so that the distal end of the electrode 266 may contact the brain tissue 111.

As previously discussed, the detection fiber 264 may carry the excitation light from the optical system 215 to the brain tissue 111 and the detection fiber 264 may also collect light from the brain tissue 111 and deliver the light to the optical system 215 which in turn provides filtered optical signals to the spectrometer 224. In such a configuration, the use of a single fiber may provide several advantages. Because the emitted light and detected light have to pass through the exact same location, the location of the tumor can be identified more precisely.

The detection fiber may have a diameter of less than 500, 450, or 400 microns. By using this diameter, the device requires the user to place the probe such that it touches the tissue of interest. By requiring the user to place the probe in contact with the tissue, the tumor can be located more precisely. If a larger diameter fiber was used, the user could detect fluorescence from a distance, but light scattering by the tissue could make it more difficult for the user to determine the precise location of the tumor.

The sample element 164 may be coupled to any surgical tool (i.e., the ultrasonic handpiece assembly 130, the suction tool 156 or the bipolar forceps 160) such that the distal end 272 of the detection fiber 264 is proximal to the working portion of the surgical tool. The distal end 272 of the detection fiber 264 may include a lens, a collimator, or another suitable optical component that allows the detection fiber 264 to deliver excitation light to the brain tissue 111 and the detection fiber 264 to collect light from the brain tissue 111.

While the example is provided that the detection fiber 264 functions to deliver excitation light to the tissue and also collect light from the tissue, the system may include two separate fibers such as a collection fiber and an excitation fiber instead. The collection fiber may collect light from the tissue and the excitation fiber may deliver excitation light to the tissue. While the detection fiber 264 and any other fibers discussed herewith are contemplated as single fibers for simplicity, it is understood that each of the fibers may include more than one fiber. For example, the detection fiber 264 may include a bundle of detection fibers all being connected in similar fashion to the single fiber connections discussed above. In another example, the detection fiber 264 may include any number of fibers connected in series.

With reference to FIG. 12B, a second alternative configuration of the sample element 164 is shown. The sample element 164′ shown is functionally equivalent to the sample element 164 shown in FIG. 12A so a detailed discussion of the functionally of equivalent parts, is hereby omitted. The indicator 298′ shown is cylinder shaped as opposed to the sphered shaped indicator 298 shown in FIG. 12A.

With reference to FIG. 12D, a third configuration for the sample element 164 is shown. The sample element 164″ shown is functionally equivalent to the sample elements 164 and 164′ shown in FIGS. 12A and 12B so a detailed discussion of the functionally of equivalent parts, is hereby omitted. In this configuration, the indicator element 296′ is provided separate from the sample element 164′ (i.e., the indicator element 296 is not integrated with the sample element 164′). The indicator element 296′″ may include a transmission member 297′, such as a wire and/or a cable covered by a jacket, an indicator 298, and a connector 299 for connecting the transmission member 297 to the indicator port 175 of the control console 168. The indicator element 296 may include a connector 299 for connecting the indicator element 296 to the indicator port 175 of the control console 168.

With reference to FIG. 12D, a fourth configuration for the sample element 164 is shown. The sample element 164′″ shown is functionally equivalent to the sample elements 164, 164′, and 164″ shown in FIGS. 12A and 12B so a detailed discussion of the functionally of equivalent parts, is hereby omitted. Here, the transmission member 297′″ of the indicator element 296′″ and the indicator 298 may be replaced with an optical fiber hereinafter referred to as an indicator fiber. The indicator fiber serves to emit light in response to detection of target tissue by the controller 204. The sample element 264′″ may also include an indicator portion 291 which is illuminated by the indicator fiber as light travels down the sample element 264′″. The indicator portion 291 may be situated proximal to the distal portion of the sample element 164 to ensure that the healthcare professional is able to view the indicator portion 291 as the healthcare professional is resecting tissue. The indicator portion 291 may be transparent or may also correspond to a removed portion of the jacket 292 of the sample element 164. The indicator fiber may be coupled to the optics block 216 via the optical connector and receive light from the excitation source 228 or another excitation source at a different wavelength than the excitation light. For example, the excitation source may generate green light (e.g., wavelengths of about 520-564 nm) when instructed by the controller 204 to indicate the detection of the target tissue.

The sample element 164′″ may include a co-axial fiber with a central core and an outer channel covered by the jacket 292. The detection fiber 264′″ may be disposed within the central core while the indicator fiber is disposed within the outer channel. A portion of the jacket 292 of the sample element 164″″ may be removed such that the indicator fiber may illuminate light through the sidewalls of the outer channel to light up the indicator portion 291.

The controller 204 may transmit an activation signal to the indicator 298 in response to the detection of the target tissue. The indicator 298 may emit light in response to receiving the activating signal. The controller 204 may control the LED to emit various colors of light depending on whether the controller 204 detects PPIX or ICG (i.e., whether the brain tissue 111 corresponds to the target tissue or a blood vessel). For example, the controller 204 may control the LED to emit green light (e.g., wavelengths of about 520-564 nm) when PPIX above a threshold is detected or yellow light (e.g., wavelengths 565-590 nm) when ICG is detected.

The spectrometer 224 is configured to convert the filtered optical signals (i.e., filtered light) into spectral signals in the form of electrical signals. The microcontroller 220 is configured to control operation of the spectrometer 224. Examples of spectrometer systems that may be used are commercially available from Hamamatsu including Mini-spectrometer micro series C12880MA. Although a spectrometer 224 is contemplated throughout the disclosure, other optical instruments may be used instead of a spectrometer 224. The spectrometer 224 may include an entrance slit, a collimating lens/mirror, transmission grating element, a focusing mirror, and an image sensor. The entrance slit may receive the collected light from the optics block 216 which then passes through the collimating lens/mirror. The collimating lens/mirror collimates the collected light passed through the entrance slit and guides it onto the grating element. The grating element separates the incident light from the collimating lens into different wavelengths and lets the light at each wavelength pass through or reflect away at a different diffraction angle. The focusing lens or mirror forms an image of the light dispersed into wavelengths by the grating element onto linearly arranged pixels of the image sensor according to wavelength.

Each wavelength is photoelectrically converted into an electrical signal (i.e., a spectral signal). The image sensor outputs the signal of light incident on each pixel at a certain time interval (i.e., the image sensor converts the optical signals into electrical signals and outputs them). The time interval may be referred to as the integration timing. The microcontroller 220 may be configured to control operation of the spectrometer 224, for example, the integration timing based on instructions from the controller 204. The microcontroller 220 forwards the spectral signals via a communication interface (e.g., serial peripheral interface (SPI)) to the controller 204.

The controller 204 is configured to transform the spectral signals provided by the microcontroller 220 into simple/usable output variables via in real-time in order to provide the healthcare professional with an indication of presence of the target tissue within the sterile fields. The controller 204 may illuminate the indicator 298 of the sample element 164 in response to detecting the target tissue.

Since ambient light may be present in the optical signals collected at the target tissue and thus present in the spectral signals provided by spectrometer 224, the controller 204 is configured to perform one or more functions or methods of control to remove the ambient light from the spectral signals (i.e., the wavelengths associated with the ambient light) to accurately detect when the brain tissue 111 corresponds to the target tissue as evidence by the PPIX present in the target tissue.

The controller 204 may be configured to remove the ambient light from the spectral signals using any suitable method, function, or algorithm in in any suitable manner. In one example, the controller 204 may pulse the one or more excitation source 228. The controller 204 may be configured to pulse the excitation source 228 such that alternating spectral signals are collected. During a first period, the controller 204 may operate the excitation source 228 in a first illumination state (IS1) where the excitation source 228 is ON and illumining the target tissue via the detection fiber 264. During a second period of time, the controller 204 may be configured to operate the excitation source 228 in a second illumination state (IS2), where the excitation source 228 is OFF and not illuminating the target tissue via the detection fiber 264.

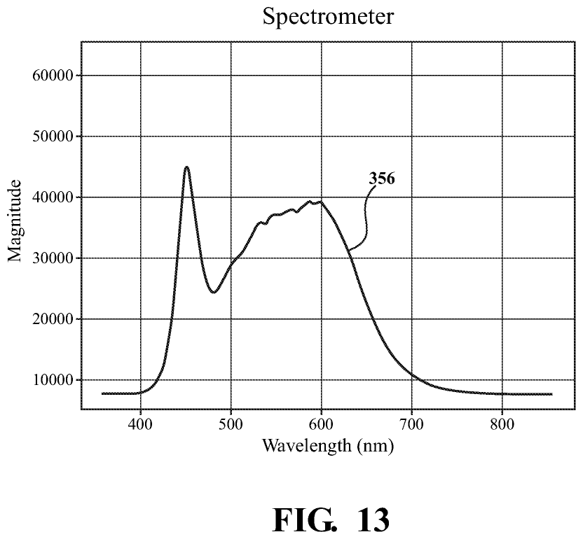

The spectral signals provided by the spectrometer 224 and generated as a result of the optical signals collected from the target tissue while the excitation source 228 is in the first illumination state (IS1) during the first period, should include the red fluorescence when the brain tissue 111 corresponds to the target tissue. The spectral signals received by the controller 204 during the first period of time while the excitation source 228 is in the first illumination state (IS1) may be referred to as excited spectral signals from this point forward. With reference to FIG. 13, an excited spectral signal 356 is shown corresponding to the red fluorescence collected during the first period of time. Due to the presence of ambient light and/or background light caused by various sources in the operating room such as the surgical microscope 108, surgical lamps, or any other devices in the operating room, the excited spectral signal 356 shows a wide range of wavelengths present in addition to the wavelengths associated with the red fluorescence. With reference to FIG. 8B, the light collected by the detection fiber 264 passes through the components 232, such as the long pass filter, of the second portion 284 of the optics block 216. After the light passes through the components 232, the light may enter the spectrometer 224 which is coupled to the optics block 216.

The spectral signals or spectrometer signals provided by the spectrometer 224 and generated as a result of the optical signals collected from the target tissue while the excitation source 228 is in the second illumination state (IS2), may contain ambient light and should not contain the red fluorescence generated by the target tissue even since the excitation light is required to be absorbed by the target in order for the tissue to emit the fluorescence. The spectral signals received by the controller 204 during the second period of time while the excitation source 228 is in the second illumination state (IS2) may be referred to as ambient spectral signals.

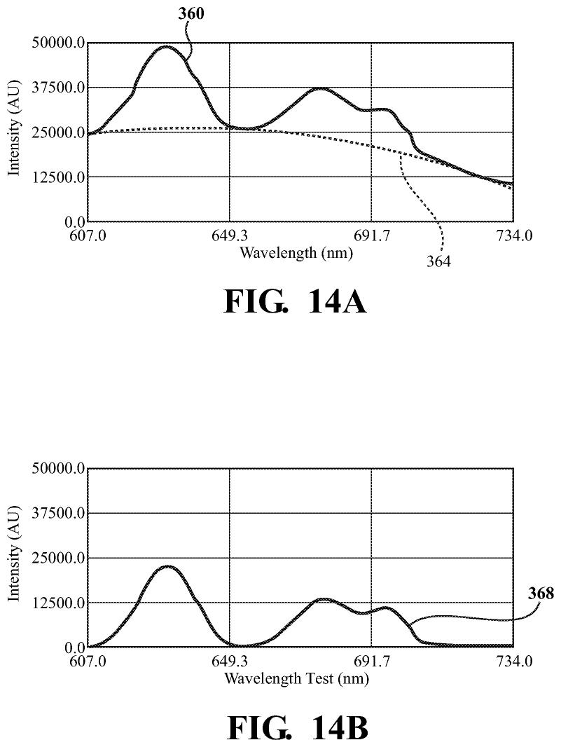

With reference to FIGS. 14A and 14B, a first modified spectral signal 360 and a second modified spectral signal 368 of the target tissue are shown. Fluorescence intensity is shown on one axis and emission wavelength is shown on the other axis. The controller 204 may be configured to generate the first modified spectral signal 360 in any suitable manner to remove ambient light (i.e., the ambient spectral signal) from consideration. For example, the controller 204 may be configured to subtract the ambient spectral signal from the excited spectral signal (i.e., subtract spectral signals provided over the second illumination state (IS2) from the spectral signals provided over the first illumination state (IS1)). After the first modified spectral signal 360 is generated, the controller 204 may be configured to further subtract any background signal still present from the first modified spectral signal 360.

The controller 204 may be configured to subtract any background signal still present in the first modified spectral signal to generate a second modified spectral signal. For example, the controller 204 may be configured to use an algorithm based on a polynomial, such as an automated polynomial fitting routine based on a modified version of least squares polynomial to obtain a baseline curve 364 representative of any background signal still present. The controller 204 using the algorithm may then subtract the baseline curve 364 from the first modified spectral signal 360 to obtain a second modified spectral signal 368 which is representative of the red fluorescence emitted from the target tissue with the ambient light and background light removed.

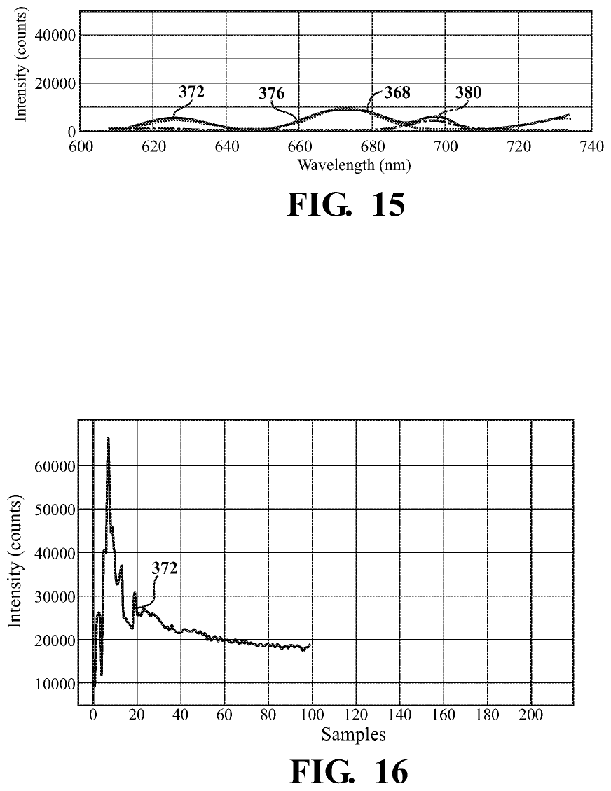

The controller 204 may be configured to fit at least one gaussian distribution/curve to the spectral signals. The controller 204 may fit the at least one gaussian distribution to raw spectral signal (i.e., the excited spectral signals and/or the ambient spectral signals), the first modified spectral signal, or to the second modified spectral signal. This may enable a level of confidence to be determined based on the results of the fitting. In FIG. 15, three gaussian curves (372, 376, 380) were fitted to the three remaining spectral bands of the second modified spectral signal 368 (i.e., the spectral signal remaining after the ambient light and the background light were removed).

With reference to FIG. 16, the controller 204 may be configured to select the gaussian band that has been fitted to PPIX's emission band (i.e., a band including 635 nm) and generate the selected band 372 for display in real time such that the healthcare professional may view the PPIX intensity in real time as the sample element 164 collects the samples. The controller 204 may store a predetermined intensity threshold that has been associated with the target tissue.

The controller 204 may be configured to generate an activation signal based on a comparison of the PPIX intensity, for the PPIX emission band that was fitted to the gaussian band, to the predetermined intensity threshold. In response to the PPIX intensity exceeding the threshold, the controller 204 may generate an activation signal. Based on the activation signal, the indicator 298 of the sample element 164 may emit light thereby providing a real-time indication to the healthcare professional of the presence of target tissue.

The controller 204 may be configured to perform an error correction process prior to generating the activation signal. During the error correcting process, the controller 204 may be configured to determine a ratio of any of the spectral signals (raw spectral signal, the excited spectral signals, the ambient spectral signals, the first modified spectral signal, or the second modified spectral signal) to a gaussian band such as the gaussian band that has been fitted to the PPIX emission band. The controller 204 may be configured to calculate at least two full width at half maximum (FWHM) points for the gaussian band. The controller 204 may be configured to calculate how far the at least two FWHM points are from the any of the spectral signals (as a percentage of their intensity). When the ratio is above a threshold (e.g., 2 percent), the controller 204 may be configured to return that the PPIX intensity falls below the threshold and thus the controller 204 does not generate the activation signal even though the activation signal would have been generated prior to the error correction process being performed.

In parallel, another error check in parallel may be performed that looks at the error between the spectrometer signal to be fitted and the results of the band-fitting process. This error check may only be calculated at specific points along the spectrometer signal, such determining the intensities as at the center and the outer most points of the band—the allowed width. This intensity may be compared with and is then compared to the amplitude of the fitted band which results in a percentage error which is compared to a predefined value. This allows the system to be more specific when deciding if the final fit band is acceptable and reduces the number of false positives-indications where the indicator indicates that the tissue is tumorous, where in reality, the tissue is not tumorous.

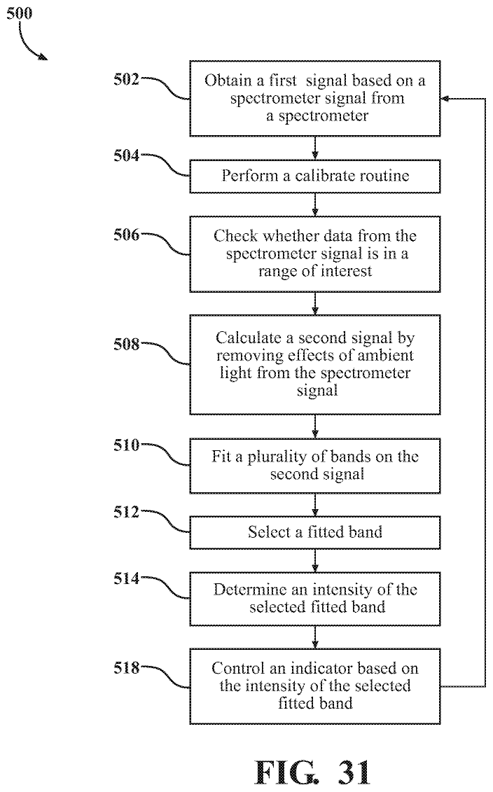

As mentioned above, the controller 204 is configured to perform one or more functions or methods of control to remove the ambient light from the spectral signals (i.e., the wavelengths associated with the ambient light) to accurately detect when the brain tissue 111 corresponds to the target tissue as evidenced by the PPIX present in the target tissue. The spectral signals or spectrometer signals provided by the spectrometer 224 and generated as a result of the optical signals collected from the target tissue while the excitation source 228 is in the second illumination state (IS2) may contain ambient light. FIG. 31 illustrates an exemplary flowchart 500 for differentiating brain tumor tissue from healthy brain tissue according to the teachings of the present disclosure. As will be appreciated from the subsequent description below, this flowchart merely represents an exemplary and non-limiting sequence of blocks to describe a flowchart for differentiating brain tumor tissue and is in no way intended to serve as a complete functional block diagram of all of the steps.

At step 502, the controller is configured to obtain a first signal based on a spectrometer signal from the spectrometer 224 and, at step 504, perform a calibrate routine if necessary. The controller 204 may be configured to perform the calibrate routine as described in greater detail below. In some configurations, the calibrate routine may include multiplying by calibration values. It is contemplated that the calibrate routine may be separate and optional. At step 506, the controller is configured to check whether data from the spectrometer signal and/or the first signal is in a range of interest. To do so, the controller 204 may be configured to check if any data (such as pixels or values) are saturated. At step 508, the controller 204 is configured to calculate a second signal by removing the effects of the ambient light from the spectrometer signal. In some configurations, to remove the effects of the ambient light, the controller is configured to discard data that are not within the range of interest.

At step 510, the controller 204 is configured to fit a plurality of bands on the second signal. In other configurations, the controller 204 may fit only a single band. At step 512, the controller 204 is configured to select a fitted band. The controller 204 may also select a fitted band from the plurality of fitted bands one or more parameters of the fitted bands such as based on a mean value criterion, a standard deviation criterion, maximum values, minimum values, band width criterion or combinations thereof. The controller is configured to select a fitted band that is associated with the fitted to the PPIX emission band. For example, the controller is configured to select a fitted band that exhibits a mean value criterion of 635 nm. In another example, the controller is configured to select a fitted band from a plurality of fitted bands based on the mean value criterion and a standard deviation criterion. The standard deviation criterion may vary, such as within 1 or 2 standard deviations.

More specifically, the controller 204 may fit a plurality of distribution curves on the second signal with the distribution curves being further defined as a plurality of gaussian distribution curves, a plurality of Lorentzian distribution curves, or combinations thereof. Other types of known distributions may also be used for band fitting. Alternatively still, the distribution curves may be fitted using other types of regression modeling.

At step 514, the controller may also determine an intensity of the selected fitted band and control the indicator 298 based on the intensity of the selected fitted band. Once selected, in some configurations, the controller 204 is configured to control the indicator 298 based on the intensity of the selected fitted band and a predetermined threshold. For example, in some configurations, the predetermined threshold may be an intensity threshold.

The controller 204 may further be configured to calculate an amplitude offset based on an amplitude of the second signal and the selected fitted band and control the indicator 298 based on the amplitude offset and an amplitude offset threshold—the second signal is the result of the spectrometer signal after windowing and after removing the effects of ambient light on the spectrometer signal. In these configurations, the controller is configured to calculate the amplitude offset based on the center of the selected fitted band.

In some configurations, the controller is configured to calculate the second signal by fitting a baseline polynomial curve to the spectrometer signal and subtract the baseline polynomial curve from the spectrometer signal to remove artifacts of ambient light to yield the second signal.

At step 516, once the intensity of the selected fitted band has been determined, the controller 204 is configured to control the indicator 298 based on the intensity of the selected fitted band. Generally, as one example, the controller 204 is configured to calculate a signal by removing the effect(s) of ambient light from the spectrometer signal, identify a single band associated with PPIX based on the second signal, and control the indicator 298 based on the intensity of the single band. In some configurations, the single band may be a band corresponding to emission of a single fluorophore. In other configurations, the single band may be a band corresponding to PPIX based on the spectrometer signal. As another example, the controller 204 is configured to calculate a signal by removing the effect(s) of ambient light from the spectrometer signal, identify at least two bands associated with PPIX based on the second signal, and control the indicator 298 based on the intensity of the plurality of bands. In some configurations, the at least two bands may be bands corresponding to emission of a single fluorophore. In other configurations, the controller may be used for fluorophores other than PPIX.

The steps 502-516 of the method 500 may be repeated, thus creating a loop that is only broken if desired. It is noted that some steps are described as being performed “if necessary.” This is intended to indicate that those steps may be omitted or optional.

The controller 204 may communicate with the ultrasonic surgical console via a communication link established through the electrical port 174. For example, a cord may be plugged into the electrical port and also plugged into the ultrasonic control console 128 to establish the communication link. The communication link may also be established wirelessly. The controller 204 may inform the ultrasonic control console 128 based on a type of tissue detected. The controller 204 may inform the ultrasonic control console 128 when target tissue is present or absent. Based on the information provided from the controller 204, the ultrasonic control console 128 may adjust one or more operating parameters. For example, when target tissue is present, the resection rate may not be limited; however, when target tissue is not present, the resection rate may be limited such that the ultrasonic surgical handpiece is prevented from cutting the healthy tissue. In such an example, the ultrasonic console may control the drive signal, such as the voltage, current, or both supplied to the ultrasonic handpiece based on the whether the target tissue is detected. While the example is provided that the controller 204 may communicate with the ultrasonic control console 128, the controller 204 may alternatively communicate with the surgical control console 115 to control the various surgical tools (e.g., bipolar forceps 160, neuro stimulators, dissectors, ablation devices, etc.) based on the absence or presence of target tissue.

The controller 204 may be configured to perform one or more standardization routines and/or calibration routines. The controller 204 prompt the healthcare professional via the user interface 208 to perform the calibration routine at the beginning of the resection procedure to account for autofluorescence variations of brain tissue 111 from person to person. The controller 204 may instruct the healthcare professional to collect light from known healthy brain tissue 111 with the detection fiber 264 of the sample element 164 to use as a standard baseline. Based on the characteristics of the light collected, the controller 204 may adjust one or more parameters of an algorithm for determining whether brain tissue is tumorous or not such as the predetermined intensity threshold for PPIX.

In a standardization routine, during a first period of time, the controller 204 may instruct the healthcare professional to collect light from a light source (e.g., a nearby light) outputting light from a consistent spectral band with the detection fiber 264 of the sample element 164. After the optical system 215 has converted the light collected into an electrical signal (hereinafter, referred to as a first standardization electrical signal), the controller 204 may store the first standardization electrical signal representative of the characteristics of the light collected. During a second period of time occurring after the first period of time, the controller 204 may instruct the healthcare professional to collect light from the same light source. After the optical system 215 has converted the light collected into a second standardization signal, the controller 204 may compare the first standardization signal obtained during the first period of time to the second standardization signal obtained during the second period of time and use the results to account for any variations of the optical readings over time. For example, the controller 204 may adjust one or more parameters of an algorithm used to determine whether the brain tissue is tumorous or not or one or more settings of the spectrometer 224 to account for any variations of the optical readings over time.

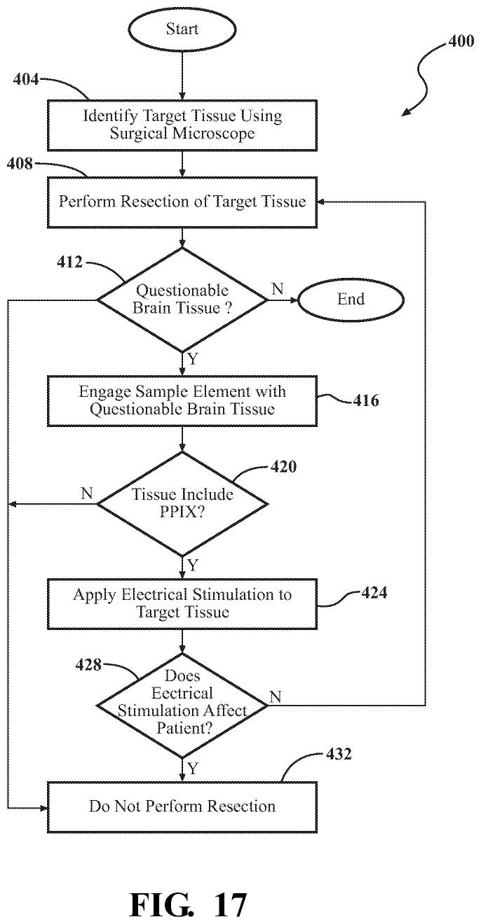

Referring back, FIG. 17 includes a flowchart 400 illustrating a surgical resection procedure in accordance with the teaching of the present disclosure. As will be appreciated from the subsequent description below, this flowchart merely represents an exemplary and non-limiting sequence of blocks to describe a typical resection procedure performed to resect target tissue and is in no way intended to serve as a complete functional block diagram of all of the steps of a resection procedure.

The resection procedure 400 begins at 404 where the healthcare professional may identify target tissue using the surgical microscope 108 under excitation light. At 408, after the target tissue has been identified, the healthcare professional may perform resection of the target tissue using one of the surgical tools described above. At 412, after resection of the target tissue identified via the surgical microscope, the healthcare professional determines whether there is any questionable brain tissue (e.g., tissue which does not emit visible light when viewed from the surgical microscope 108 under excitation but has characteristics associated with the target tissue) that may correspond to target tissue. If there is no questionable brain tissue, the resection procedure may end; otherwise, the resection procedure continues at 416.

At 416, the healthcare professional engages the sample element 164 with the questionable brain tissue (e.g., to excite the brain tissue and collect light from the brain tissue). At 420, the healthcare professional determines whether the tissue includes PPIX as evidenced by the indicator 298 of the sample element 164. If so, the resection procedure continues at 424; otherwise, the resection procedure continues at back at 412. At 424, the healthcare professional applies electrical stimulation to the target tissue. At 428, the healthcare professional determines whether the electrical stimulation affected the patient. If so, the healthcare professional may choose not to perform resection of the target tissue; otherwise, the resection procedure continues back at 408.

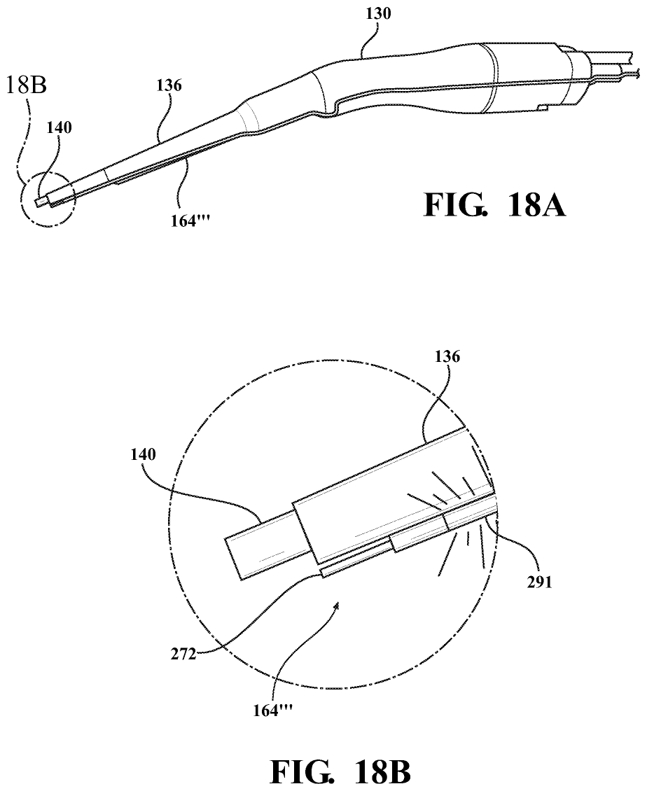

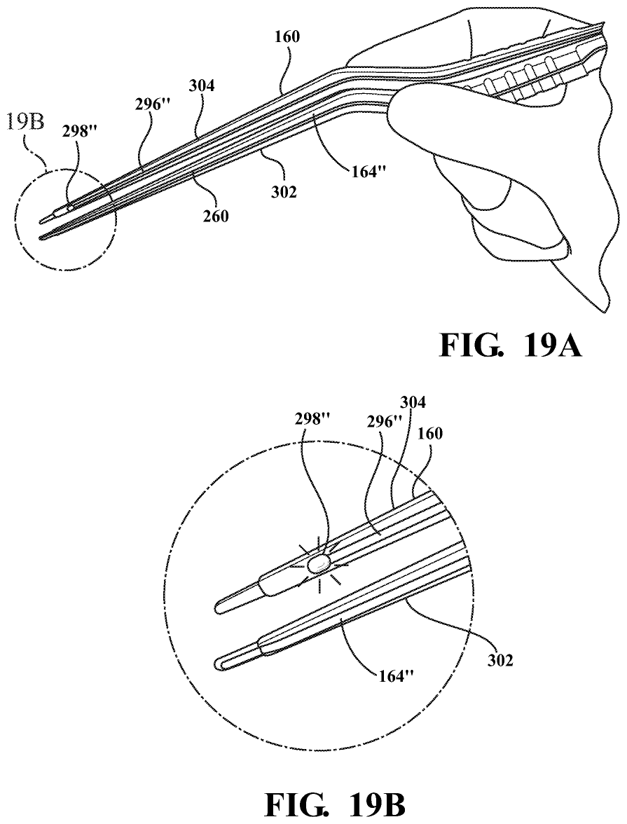

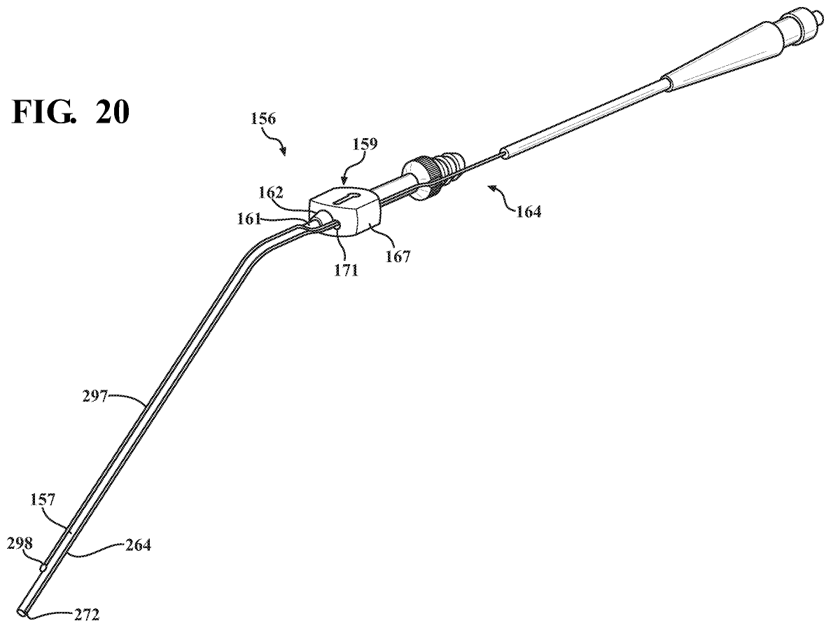

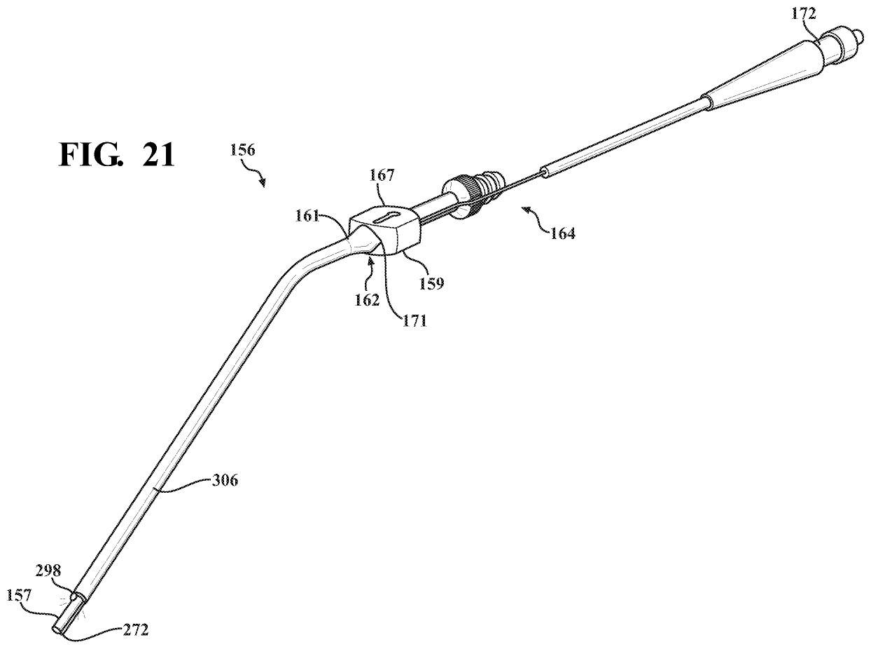

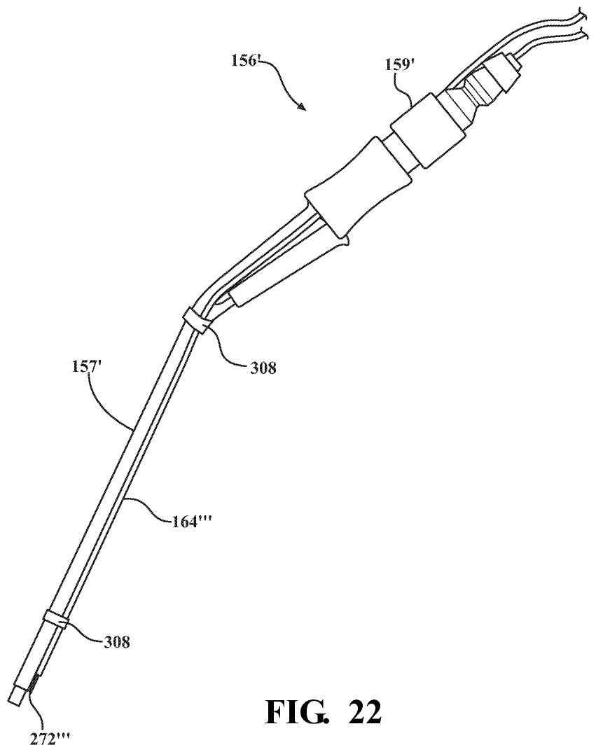

With reference to FIGS. 18-21, the sample element 164 may be coupled to any surgical tool. The sample element 164 may be coupled to the ultrasonic handpiece assembly 130 as shown in FIGS. 18A and 18B, to the bipolar forceps 160 (or any surgical tool associated with the surgical system 112 such as dissector, etc.) as shown in FIGS. 19A and 19B, and the suction tool 156 as shown in FIGS. 20-22. The sample elements 164 and 164′ and/or the indicator element 296 may be coupled to the surgical tools in any suitable manner. For example, the sample element 164 (or the sample element 164″ and indicator element 296″) may be coupled to the surgical tools via an adhesive. The adhesive may be in the form of a sticker or substance such as glue. Additionally, or alternatively, the sample element 164 (or sample element 164″ and indicator element 296″) may also be coupled to the surgical tools via a fixation element discussed in greater detail with respect to FIG. 22 or a jacket discussed in greater detail with respect to FIGS. 21 and 22.

As shown in FIG. 18B, the sample element 164′″ may be coupled to the ultrasonic handpiece assembly 130 in any manner as long as there is no direct contact between the tip 140 and distal portion of the sample element 164′″. For example, the sample element 164′″ may terminate at a portion of the sleeve 136 proximal to the tip 140. In another example, the sample element 164′″ may extend past the sleeve 136 but be arranged such that there is adequate empty space between the tip 140 and the sample element 164′″ to prevent contact between the tip 140 and the sample element 164′″.

In the configuration shown in FIGS. 19A and 19B, the sample element 164″ is shown coupled to an outer portion of a first pincer 302 the bipolar forceps 160 and the indicator element 296″ is shown coupled to an inner portion of a second pincer 304 of the bipolar forceps 160. As shown the indicator is disposed near the tip of the second pincer such that the healthcare professional can view the indicator 298″ while performing resection of the target tissue without having to look at another screen or portion of the tool.

With reference to FIGS. 20, 21, and 23, the sample element 164 is shown coupled to the suction tool 156. The detection fiber 164 and a portion of the indicator element 296, (i.e., the transmission element 297 and indicator 298) may be guided through the through bore 171 of the handle 159. A distal end 272 of the detection fiber 264 may be positioned proximally to a distal end of the suction cannula 157. The indicator 298 may be positioned near the distal end of the detection fiber 264 but more proximal to a distal end 162 of the control portion 167 of the handle 159 than the distal end 272 of the detection fiber is. In other words, the distal end 162 of the detection fiber 264 may be disposed more proximal to the distal end of the suction cannula 157 than the indicator 298 is. With additional reference to FIG. 21, after the detection fiber 264 and the portion of the indicator element 296 is fed through the through bore 171, a jacket 306 may be fitted over top of the suction cannula 157, the detection fiber 164, and the transmission element 297. The jacket 306 may be mated to the distal end 162 of the handle 159 so that the distal end 162 and the through bore 171 are covered. The jacket 306 may terminate just before where in the indicator 298 is coupled to the suction cannula 157. The detection fiber 264 may protrude from beneath the jacket 306 so that the jacket 306 does not interfere with the delivery of excitation light or collection of fluorescence from the tissue. Also as shown, the indicator 298 is exposed fully but may be partially covered by the jacket 306. In some configurations, the jacket 306 may be omitted.

With reference to FIG. 22, a different configuration of a suction tube 156′ is shown. Specifically, the suction tube 156′ does not include a through bore in a handle 159′ of the suction tube 156′. Instead, the sample element 164′″ is coupled to the suction tool 156′ via fixation elements 308. Specifically, the sample element 164′″ is shown coupled to the suction cannula 157′ by two fixation elements. Although only two fixation elements 308 are illustrated, more than two fixation elements 308 may be used to couple the sample element 164 to the suction cannula 157′ or the handle 159′. The fixation elements 308 may include a clip, a band, or anything that may secure the sample element 164 to the suction tool 156′.

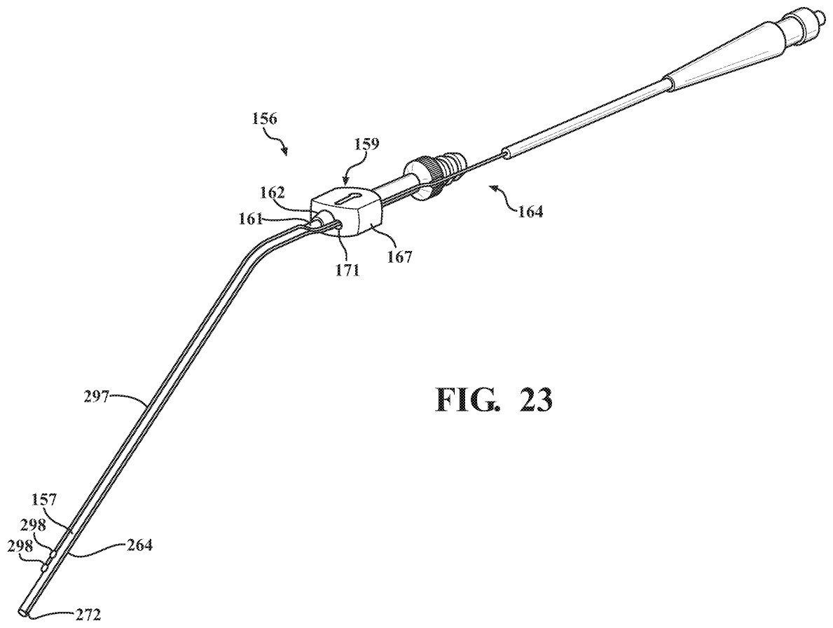

With reference to FIG. 23, the transmission member 297 may be connected to a second indicator 299. The second indicator 299 may include one or more light emitting diodes or another suitable light source. The second indicator 299 may be situated proximal to the distal portion of the sample element 164 and/or relative to the indicator 298 to ensure that the healthcare professional is able to view the second indicator 299 as the healthcare professional is resecting tissue. The second indicator 299 is configured to emit light to indicate the sample element 164 and/or the suction tube 156 are properly functioning and/or connected. In some configurations, the second indicator 299 may emit orange light (e.g., wavelengths of about 585-620 nm). In this way, the healthcare professional is able see whether there is a detection of tumorous or target tissue via the indicator 298 and whether the sample element 164 and/or the suction tube 156 is properly functioning or connected via the second indicator 299 as well as be able to readily distinguish between the colors of the indicator 298 and the second indicator 299.

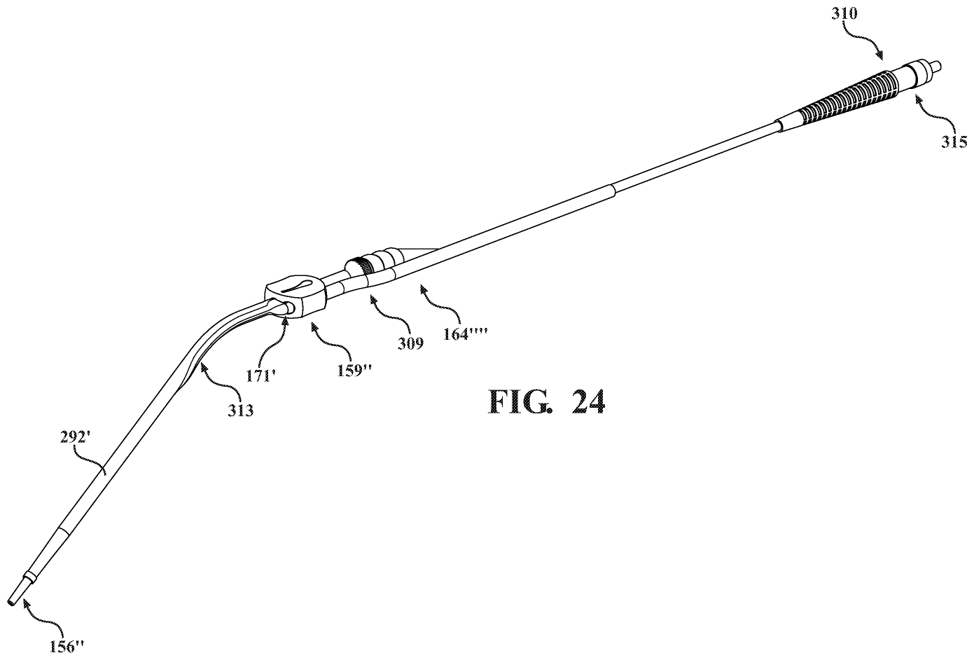

With reference to FIGS. 24-27B, a different configuration of a sample element 164″″ coupled to a suction tube 166″ is shown. Specifically, the suction tube 166″ includes a through bore 171′ in a handle 159″ of the suction tube 166″. The sample element 164″″ shown is functionally equivalent to the sample elements previously shown throughout the Figures so a detailed discussion of the functionally of equivalent parts, is hereby omitted. A connector line 309 may be guided through the through bore 171′ of the handle 159″. The connector line 309 may include the cable 144 or any other cable and/or power cord. For example, the connector line 309 may comprise a power connector or adapter configured to couple the suction tube 166″ to a power supply. In some configurations, the detection fiber 164 and a portion of the indicator element 296, (i.e., the transmission element 297 and indicator 298) may be guided through the through bore 171′ of the handle 159′. A distal end 272 of the detection fiber 264 may be positioned proximally to a distal end of the suction tube 166″.

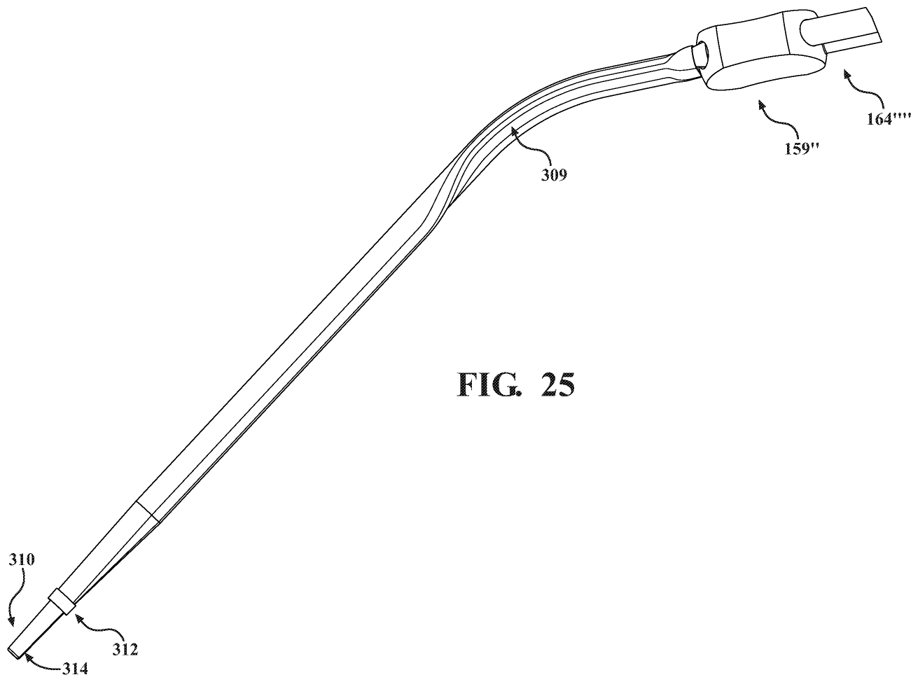

With additional reference to FIG. 24, the sample element 164″″ is shown covered by a jacket 292′. The jacket 292′ may be fitted over top of the suction tool 156″ or a portion of the suction tool 156″, a detection fiber associated with the sample element 164″″, and a transmission element associated with the sample element 164″″. In some configurations, the jacket 292′ may be mated to the distal end of the suction tool 156″ so that a portion of the suction tool 156″ is covered. FIG. 25 shows the suction tool 156″″ with the jacket 292′ omitted for exemplary purposes to show at least one of the connector line 309, the detection fiber, or the transmission element associated with the sample element 164″″.

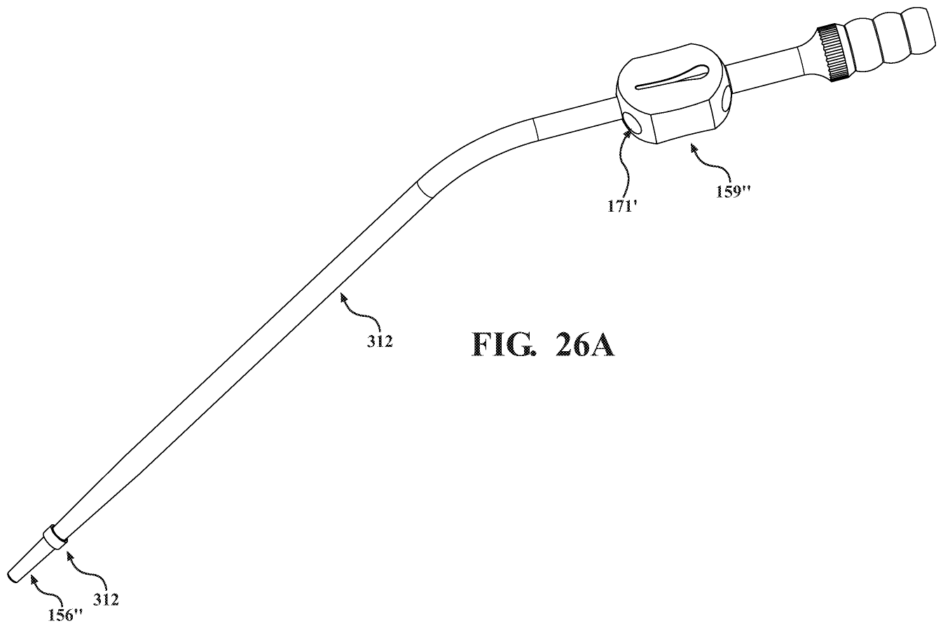

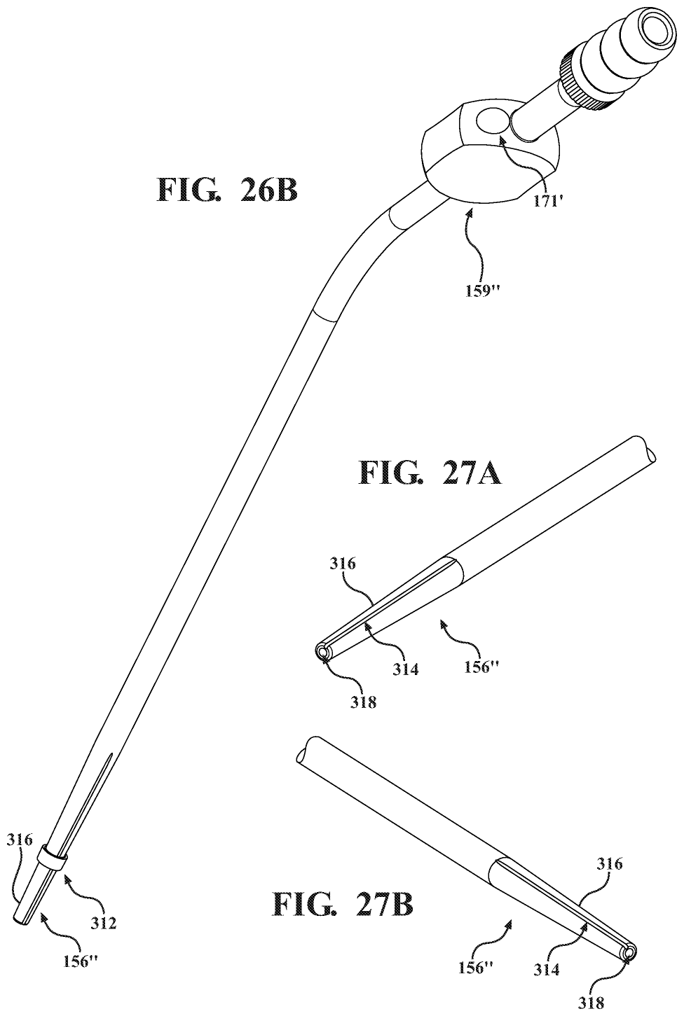

With additional reference to FIG. 25 and as shown in FIG. 26B, the outer profile of the suction tube 166″ defines an outer channel 314. The outer channel 314 of the suction tube 166″ may be situated along any appropriate or selected portion of the suction tube 166″. The detection fiber 164 may pass through the outer channel 314 and be directed toward a distal end of the suction tube 166″. In some configurations, the outer channel 314 is configured to accommodate a single optical fiber. In various configurations, the suction tube 166″ may form one or more outer channels 314. The channels, therefore, may be formed by a combination of the sample element 164, 164′, 164″, 164′″ and the suction tube 166″, or by only one or the other. It is understood the outer channel 314 may also be provided as the only or singular passage along the majority of the length of the suction tube 166″.

As shown in FIG. 27A, the outer channel 314 extends to the distal end of the suction tube 166″. The distal end of the suction tube 166″ may have a circular outer profile with an outer surface 316 and a lumen 318. The lumen defines a circular cross-section. In some configurations, the outer surface 316 of the suction tube 166″ defines the outer channel 314 to accommodate a cable, a single optical fiber. For example, in configurations where the outer surface 316 of the suction tube 166″ defines the outer channel 314, a connector 310, including the single optical fiber and the electrical terminal, is at least partially disposed within the outer channel 314. Alternatively or additionally, a tube (e.g., a heat shrink tubing) or sleeve 312 may be placed over at least a portion of the suction tube 166″ including the outer channel 314 as best shown in FIGS. 25-26B. As a consequence of the heat shrink tubing over the outer channel 314, the cable or single optical fiber accommodated by the outer channel 314 is held against the suction tube 166″ by the tube. In this way, the fiber and/or electrical conductor is secured between the suction tube 166″ and the sleeve 312. In some configurations, an electrical conductor extends through the heat shrink tubing and is in electrical communication with the indicator 298 and the electrical terminal 315. Other configurations are contemplated. In some configurations, the electrical terminal 315 is coupled to a light indicator and a single optical fiber 311 is in optical communication with a distal end of the single optical fiber 311. In such configurations, the surgical tool may include a suction line with the suction line tethered to the connector line 309.

An alternative implementation of the suction tool 156″ is shown in FIG. 27B. In this implementation, the outer channel 314 is disposed on the top of the outer surface 316 of the suction tube 166″. Referring back to FIG. 26B, the suction tube 166″ is shown bending downward and the outer channel 314 is also on the bottom of the outer surface 316. In the implementation shown in FIG. 27B, however, the suction tube 166″ bends downward while the outer channel 314 is on the top of the outer surface 316 such that the outer channel 314 is on the side opposite the direction of curvature. Other elements of the suction tool 156″ of the implementation shown in FIG. 27B may be the same as the elements of the suction tool 156″ shown in FIG. 27A.

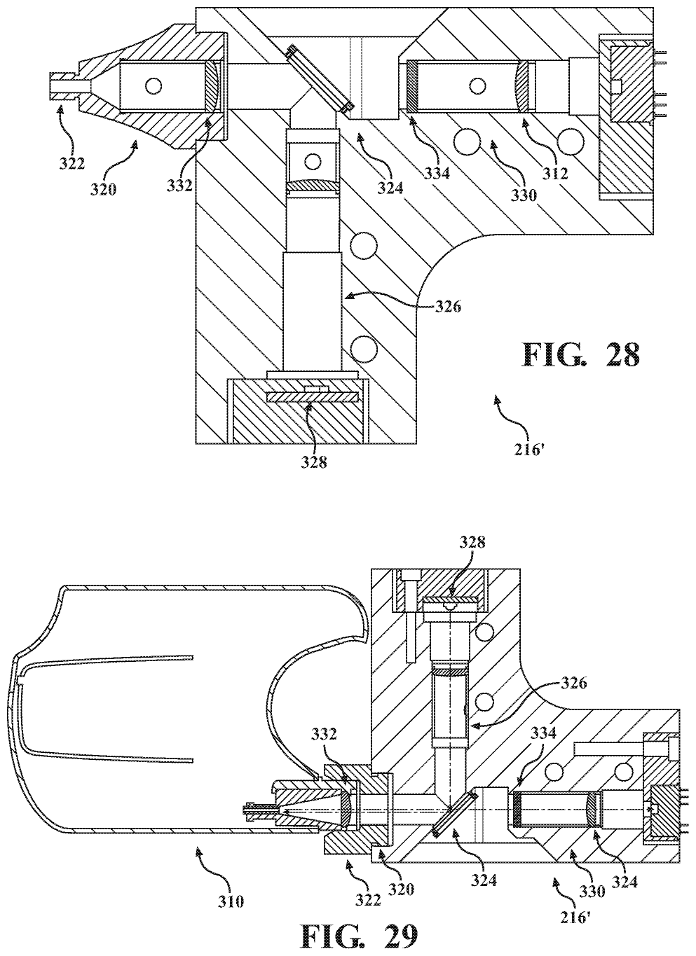

Referring to FIGS. 28 and 29, another configuration of a portion of an optics block 216′ is shown. The optics block 216′ may include an outer casing constructed of metal or another suitable material and may fully enclose components 232′ of the optics block 216′. FIGS. 28 and 29 show the optics block 216″ with a top of the casing removed such that components of the optics block 216′ are visible. The optics block 216′ defines a first portion 320 extending from a connection port 322 to a first junction 324, an emission portion 326 extending from the first junction 324 to a light emitter or excitation source 328 (as best shown in FIG. 29), and a detector portion 330 extending from the first junction 324 to the spectrometer 224. In some configurations, a fiber lens 332 is disposed in the first portion 320 and one or more spectrometer lens 324 are disposed in the detector portion 330. The light emitter 328 may be disposed in the emission portion 326.

In some configurations, the connector line 309 extends from the suction tube 166″ and the connector 310 is coupled to the connector line 309. The connector 310 includes the single optical fiber 311 and an electrical terminal 315. The connector 310 is configured to connect to a surgical console (e.g., the control console 168) including a light source or excitation source(s) and a controller (e.g., the controller 204). As shown in FIG. 29, the connector 310 may be configured to couple to the connection port 322 of the optics block 216′.

The optics block 216′ may include other optical components such as one or more mirrors, lenses, optical connectors, optical fiber, and/or any other suitable optical components. For example, in some configurations, the optics block 216, 216′ may include three lenses. In some configurations, the three lenses have identical focal lengths to transmit light from a light emitter or excitation source to a detection fiber and from the detection fiber to the spectrometer 224. While in other configurations, the three lenses may have varying focal lengths. For instance, a lens associated with the light emitter 328 or excitation sources 228 may have a focal length different from a focal length of at least one of the lenses associated with either the spectrometer 224 or the detection fiber. In another instance, the lens associated with the light emitter 328 or excitation sources 228 may have a focal length greater than either the lens associated with the spectrometer 224 or the detection fiber. In another instance, the lens associated with the light emitter 328 or excitation sources 228 may have a focal length greater than both the lens associated with the spectrometer 224 and the detection fiber. For example, the lens associated with the light emitter 328 or excitation sources 228 may have a focal length of 50 mm, the lens associated with the spectrometer 224 may have a focal length of 25 mm, and the lens associated with the detection fiber may have a focal length of 30 mm. Other focal lengths are contemplated, and it will be appreciated that the focal lengths may vary.

In some configurations, the optics block 216′ includes at least spacer, which may be configured as a c-shaped member 334 positioned between at least two lens and/or filters and/or optical components. In some configurations, the c-shaped member may alternatively be shaped as a cylinder. Other shapes and configurations are contemplated. In some configurations, the spacer 334 may be configured to act as spacers between lenses to maintain a distance or separation between the lenses. The optics block includes cylindrical channels drilled to a certain diameter. When the optical components, such as lens and/or filters, are seated in their positions within the cylindrical channels, the spacer is slid into the cylindrical channel to maintain distance/separation between the optical components. By using a c-shaped member, the c-shaped member may be compressed during insertion such that it fits snugly within the cylindrical channel. This prevents the spacer from rattling around during the housing. The c-shaped member may be made of metal or plastic.

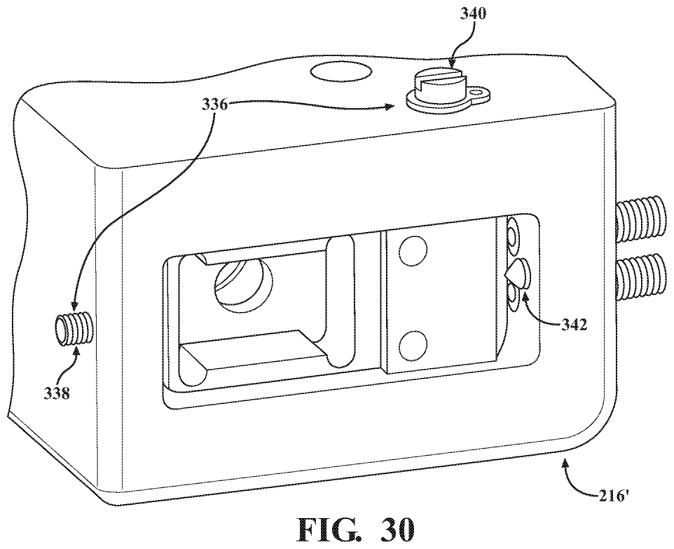

To control a position of the spectrometer 224 and/or light emitter 328 for alignment purposes, an adjustable screw assembly may be integrated into the optics block 216′. As shown in FIG. 30, an adjustment mechanism 336 to adjust the position of the spectrometer 224 and/or the light emitter 328 is provided. The adjustment mechanism 336 includes a first adjustment member 338 to adjust the position of the spectrometer 224 in a first degree of freedom and a second adjustment member 340 in a second degree of freedom. The first and second adjustment members 338, 340 may be a screw. It is contemplated that the first and second adjustment members 338, 340 may be any type of adjustable component. It is further contemplated that other features of the optics block 216′, such as additional adjustment members, are provided to align the center of the spectrometer 224 to the center of an optical beam. For example, in some configurations, the adjustment mechanism 336 further includes a third adjustment member 342 configured to lock a position of the spectrometer 224 and/or light emitter 328 once such components are aligned. In this way, the light source and sensor are aligned for optimal emission and/or collection.

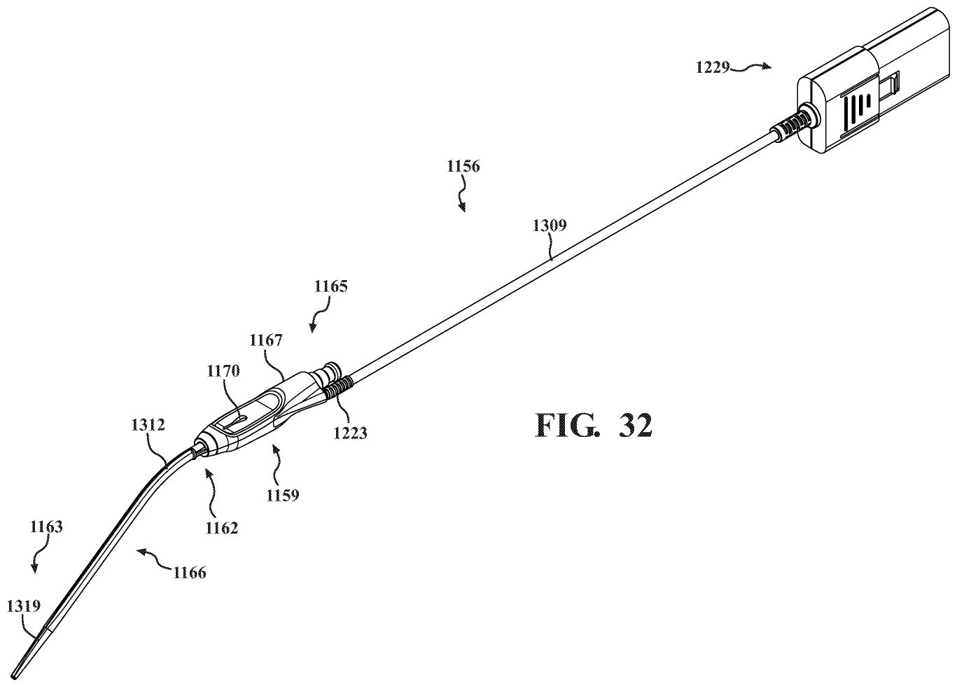

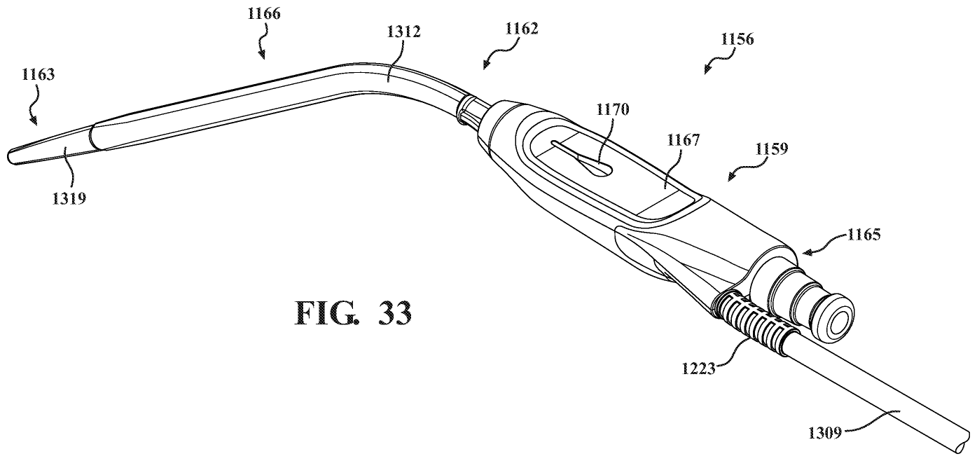

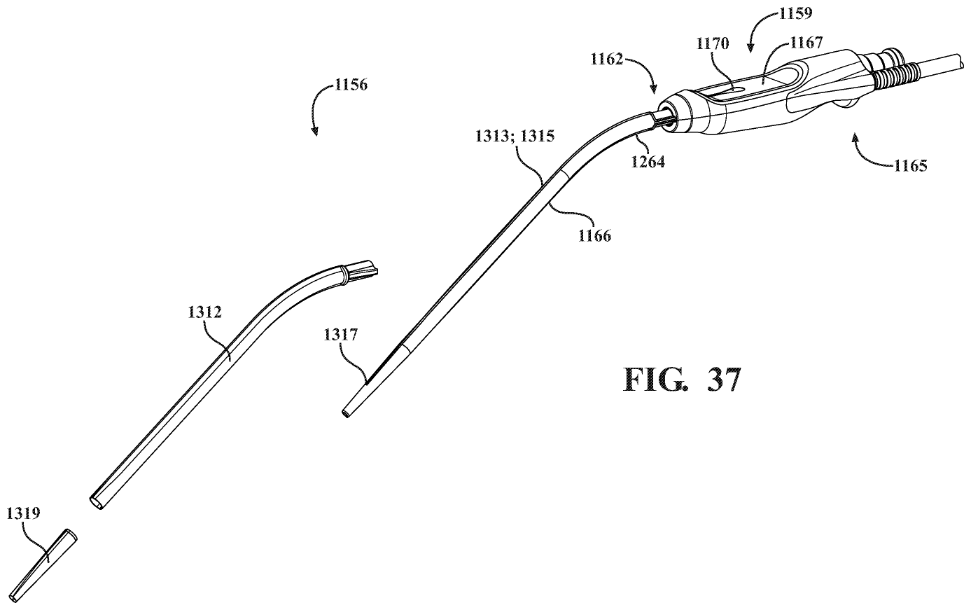

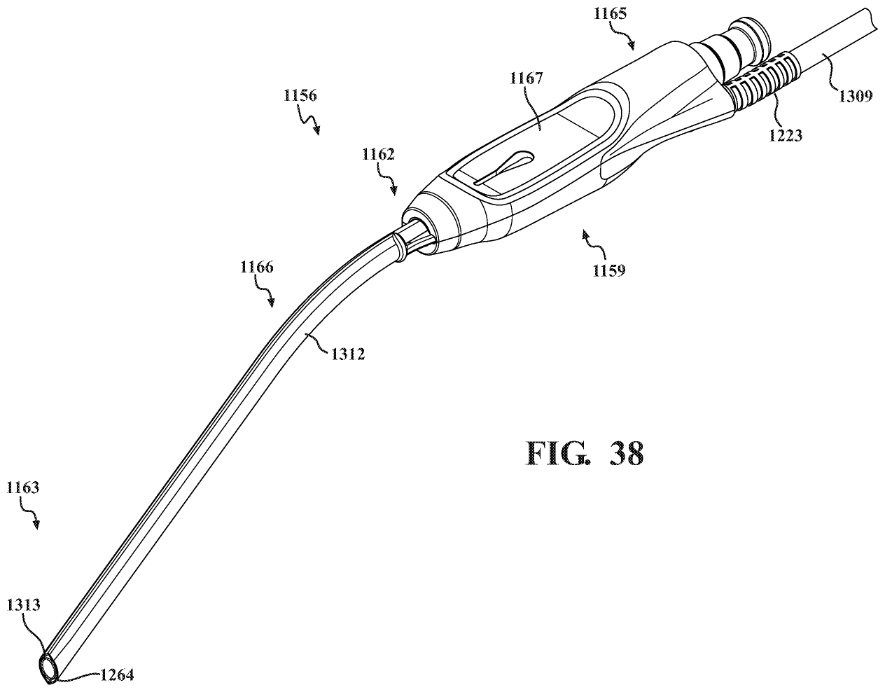

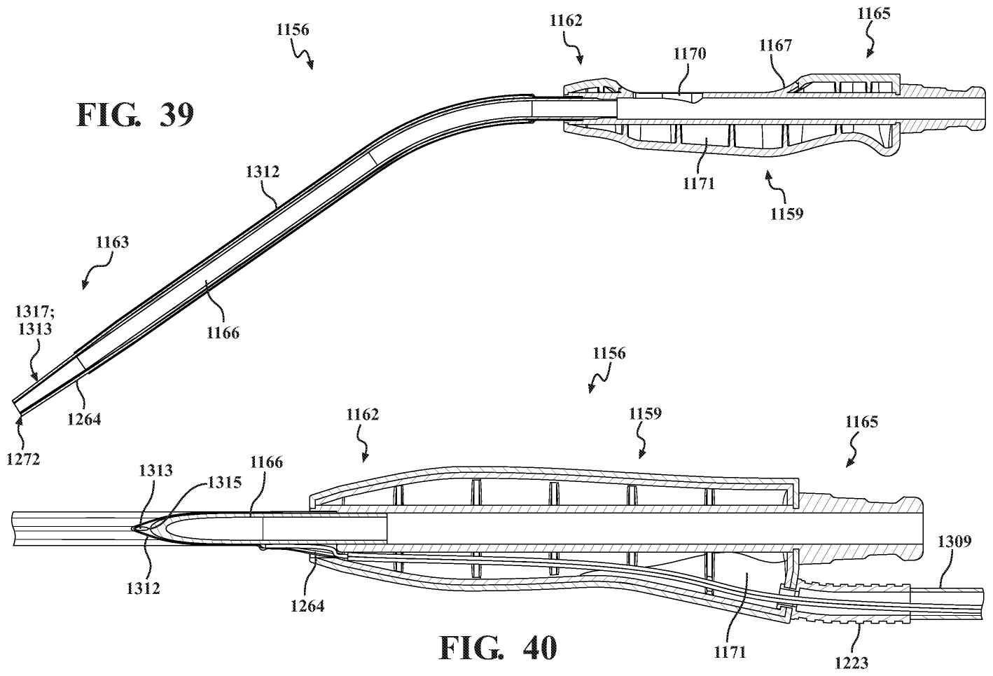

With reference to FIGS. 32-41, another different configuration of a suction tool 1156 is shown. The suction tool 1156 includes a suction tube 1166 or tubular member and a handle 1159. The suction tube 1166 defines a lumen 1318 for suctioning fluid, debris, and tissue from a patient. The handle 1159 has a control portion 1167 that may be contour shaped to allow a healthcare professional to grip the handle 1159 more easily. While the suction tool 1156 is described as having a Fukushima configuration, other configurations are contemplated such as a Frazier or Poole configuration. As shown here, the suction tube 1166 may be curved along its length in a generally downward direction. The suction tube 1166 curves downward to provide a more ergonomic grip for the surgeon operating the suction tool 1156. The suction tube 1166 may be divided approximately in the middle along its length into a top or first side and a bottom or second side. The first side generally being the side on the outside of the curve direction and the second side generally being the side on the inside of the curve direction.

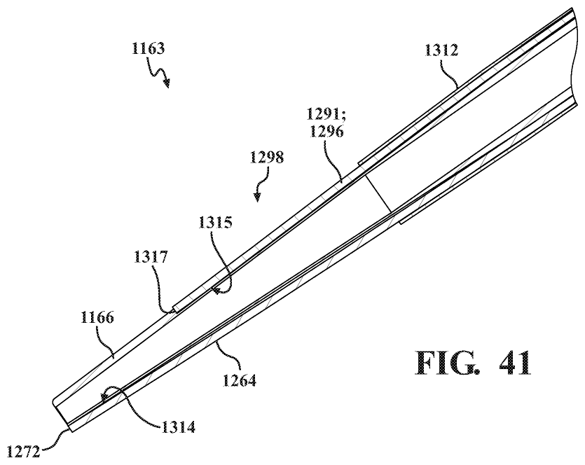

A distal end 1162 of the handle 1159 may be tapered and is configured to receive a proximal end of the suction tube 1166. A proximal end 1165 of the handle 1159 includes a vacuum fitting which may be configured to receive a suction hose, which is connected to the vacuum source that generates the suction pressure. The vacuum fitting may be a standard barbed fitting, quick disconnect, or any other suitable fitting known in the art to allow the suction hose to be fluidly coupled to a vacuum source. Specifically, the suction tool 1156 includes an internal passage 1171 (FIG. 41) in the handle 1159 of the suction tool 1156 for receiving the sample element 1264, as will be discussed in greater detail below. The sample element may be disposed in a connector line 1309, which may be guided through the internal passage 1171 of the handle 1159. The connector line 1309 may include a cable and/or power cord.

The control portion 1167 may include a teardrop shaped control 1170 for regulation of suction pressure. For example, when no portion of the teardrop shaped control 1170 is covered by the healthcare professional, suction pressure may be minimal, and when the teardrop shaped control 1170 is covered completely, suction pressure may be at its maximum. While the control portion 1167 is described as including a teardrop shaped control, the control portion 1167 may include another suitable input such as a button or different shaped control to allow the healthcare professional to vary the suction pressure. The healthcare professional holds the suction tool 1156 from its handle 1159, manipulating the suction tool 1156 so that a distal end 1163 thereof contacts the tissue of the patient during the surgical procedure in order to provide suction at the desired location.

With reference to FIGS. 32-41, another configuration of the sample element is shown and includes a detection fiber 1264 coupled to the suction tube 1166 or tubular member. The sample element shown is similar to the sample elements described above and a detailed discussion of the functionally of equivalent parts, is hereby omitted. In some configurations, the detection fiber 1264 and a portion of the indicator element 1296, (i.e., the transmission element 1297 and indicator 1298) may be guided through the internal passage 1171 of the handle 1159. A distal end 1272 of the detection fiber 1264 may be positioned proximate to a distal end of the suction tube 1166. In some implementations the detection fiber 1264 may utilize an optical fiber having a diameter of less than 500 μm.

Here, the transmission element 1297 of the indicator element 1296 and the indicator 1298 comprise an optical fiber hereinafter referred to as an indicator fiber 1313. The indicator fiber 1313 serves to emit light in response to detection of target tissue by the controller 204. As shown here, a distal end 1317 of the indicator fiber 1313 may comprise an indicator portion 1291, which may be situated proximal to the distal portion of the suction tube 1166 or other tool to ensure that the healthcare professional is able to view the indicator portion 1291 as the healthcare professional is resecting tissue. The indicator fiber 1313 may be coupled to the optics block 216, 216′ via an optical connector 1229 and receive light from the excitation source 228 or another excitation source, such as a different light source, at a different wavelength than the excitation light. For example, the light source coupled to the indicator fiber may generate green light (e.g., wavelengths of about 520-564 nm) when instructed by the controller 204 to indicate the detection of the target tissue, such as tumorous tissue. In some implementations the indicator fiber 1313 may utilize an optical fiber having a diameter of less than 500 μm. Here, the indicator portion 1291 of the indicator fiber 1313 may comprise a textured surface for scattering transmitted light. The textured surface reduces the internal reflections through the fiber and allows light to shine through the outer radial surface of the indicator fiber 1313. The textured surface is formed by deforming the outer surface of the indicator fiber 1313 via a crimping process. The crimping process causes the otherwise regular and smooth outer surface to become textured, thereby allowing light reflected along the indicator fiber 1313 to shine through.

Other processes for forming the textured surface are also contemplated, and various textures are contemplated, such as threads, radial cuts, axial cuts, and combination thereof. It should be appreciated that a portion of the core portion and a portion of the cladding layers may include the textured surface, such that the light can transmit radially through the jacket. Further details are described in U.S. Pat. No. 9,067,050, which is hereby incorporated by reference in its entirety.

As mentioned above, and best shown in FIG. 40, the detection fiber 1264 and the indicator fiber 1313 may be routed through the handle 1159 of the suction tool 1156 and down the length of the suction tube 1166. The optical fibers 1264, 1313 are received inside the handle 1159 at a proximal end 1165 and exit at the distal end 1162. A strain relief feature 1233 is arranged around the optical fibers 1264, 1313 and engages the handle 1159 to limit the bend radius of the optical fibers 1264, 1313 where they meet the proximal end 1165 of the handle 1159. The optical fibers 1264, 1313 exit the proximal end 1165 of the handle 1159 at an angle relative to a longitudinal axis of the suction tool 1156. This angled arrangement allows a straighter path for vacuum through the handle 1159 while arranging the optical fibers 1264, 1313 out of the way of the surgeon to prevent tangling and clutter.

As mentioned above, the indicator 1298 may be a visual indicator configured to emit light when the controller 204 determines the intensity of the single band associated with PPIX and based on the signal of collected light with the effect of ambient light removed is above a predetermined threshold. The light emitted is ideally in the visible spectrum and contrasting with the tissue around the suction tube 1166. For example, the light emitted may be orange or amber in color. In some cases, the light emitted from the indicator 1298 may be a single brightness for the surgeon to determine an on or off condition of the indicator 1298. Alternatively, the indicator 1298 may emit light at a brightness corresponding to the intensity of the single band. Said differently, when the intensity of the single band is exactly equal to the predetermined threshold, the indicator 1298 may emit only a small amount of light, and conversely, when the intensity is very high, the indicator 1298 will emit more light. As the intensity of the single band increases the brightness of the indicator 1298 increases in a corresponding manner. In one aspect the visual indicator may emit light in a flashing or pulsing pattern. The speed or pattern of the pulsing may vary according to the intensity of the single band. In such instances, a light source coupled to the indicator fiber would cause the indicator fiber to emit light or brightness according to the intensity of the single band.

In another implementation the indicator 1298 may be an audible indicator configured to emit sound when the intensity of the single band associated with PPIX and based on the signal of collected light with the effect of ambient light removed is above a predetermined threshold. In one aspect the sound emitted from the audible indicator may change in pitch according to the intensity of the single band. In another aspect the sound may comprise an alternating or pulsing tone that changes speed based on the intensity. Said differently, the sound may comprise two tones that alternate slowly for a single band intensity at or near the predetermined threshold and alternate more quickly as the single band intensity increases. In yet another aspect the sound emitted may vary in volume according to the intensity of the single band. Some implementations of the indicator 1298 may utilize the audible indicator instead of the visual indicator. Other implementations of the indicator 1298 may utilize the audible indicator in addition to the visual indicator. The audible indicator may take the form of a speaker that is integrated with the console, or integrated into a display device, or connected, directly or indirectly to the console via a wired or wireless connection

Other implementations of the indicator 1298 may include visual indicators that are positioned for the surgeon to see but not coupled to the suction tool 1156. For example, a surgeon may be wearing an extended reality headset to assist in performing a neurosurgery procedure using augmented reality, alternative reality, or mixed reality. In one implementation the surgeon may view the surgical site in augmented reality, and indicator may display an overlaid visual indicator in the surgeon's field of view when the intensity of the PPIX band is above the predetermined threshold. In one aspect the augmented indicator could be a virtual light positioned at a distal end of the suction tube 1166. In another aspect the augmented reality indicator could act as a paint brush using machine learning and artificial intelligence identify boundaries of the suction tube's position and virtually alter the color of the fluorescing tissue as displayed to the surgeon.

In general, the distal end 1272 of the detection fiber 1264 functions as a light detecting portion of an optical probe. The optical probe is configured to be positioned adjacent to tissue of interest. As such, the distal end 1272 of the detection fiber 1264 may be positioned proximate to a distal end of the suction tube 1166. The indicator 1298 may be positioned near the distal end 1272 of the detection fiber 1264 but more proximal to a distal end 1162 of the control portion 1167 of the handle 1159 than the distal end 1272 of the detection fiber 1264 is. In other words, the distal end 1272 of the detection fiber 1264 may be disposed more proximal to the distal end of the suction cannula 1157 than the indicator 1298 is. In some cases, the distal end 1272 of the detection fiber 1264 may be aligned with the distal end of the suction tube 1166. That is to say that the indicator 1298, such as the distal end of the indicator fiber, may be arranged adjacent to the distal end 1272 of the detection fiber 1264.

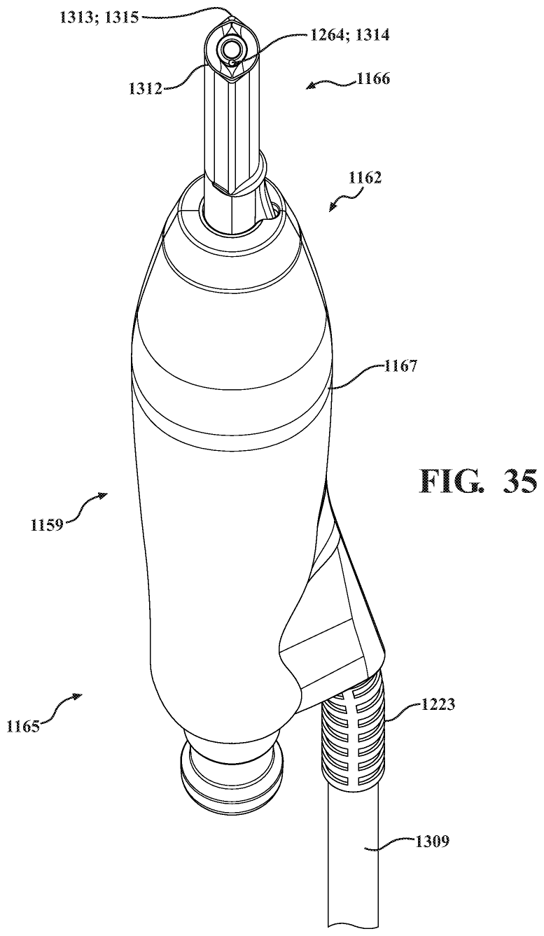

Turning now to FIGS. 35 and 41, the outer profile of the suction tube 1166 may define a first outer channel 1314 and a second outer channel 1315. In the implementation shown here, the first outer channel 1314 of the suction tube 1166 may be situated or arranged on the first side of the suction tube 1166. Similarly, the second outer channel 1315 may be situated or arranged on the second side of the suction tube 1166. Said differently, the first outer channel 1314 is arranged on the bottom side of the suction tube 1166 and the second outer channel 1315 is arranged on the top side of the suction tube 1166.

The detection fiber 1264 may pass through the first outer channel 1314 and be directed toward a distal end of the suction tube 1166. In the configuration shown here, the first outer channel 1314 is configured to accommodate a single optical fiber. In other configurations (not shown), the first outer channel 1314 may be configured to accommodate two optical fibers, for example the detection fiber 1264 and the indicator fiber 1313. In this way both the detection fiber 1264 and the indicator fiber 1313 are at least partially disposed within the first outer channel 1314.

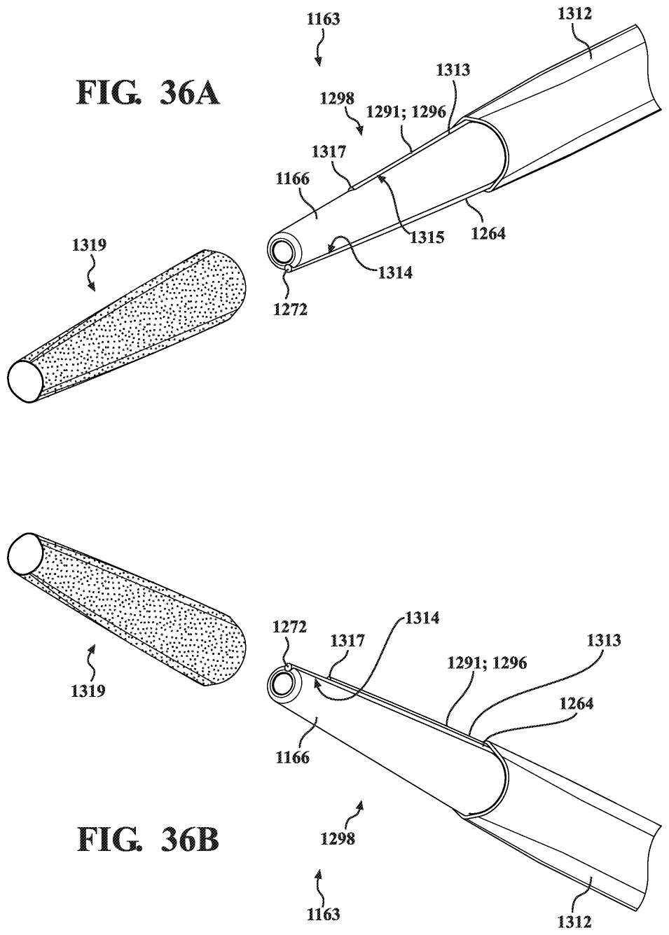

As shown in FIG. 36A, the first outer channel 1314 extends to the distal end of the suction tube 1166. The distal end of the suction tube 1166 may have a circular outer profile with an outer surface 1316 and a lumen 1318. The lumen 1318 defines a circular cross-section. In some configurations, the outer surface 1316 of the suction tube 1166 defines the first outer channel 1314 to accommodate the detection fiber 1264. For example, in configurations where the outer surface 1316 of the suction tube 1166 defines the first outer channel 1314, the first outer channel 1314 extends to the distal end of the suction tube 1166.

The indicator fiber 1313 may pass through the second outer channel 1315 and be directed toward a location that is proximal of the distal end of the suction tube 1166. Said differently, the indicator fiber 1313 is at least partially disposed within the second outer channel 1315 and the second outer channel 1315 extends along the length of the suction tube 1166 terminates prior to the distal end of the suction tube 1166.

As mentioned above, the distal end 1272 of the detection fiber 1264 and the distal end 1317 of the indicator fiber 1313 may each be positioned at or near a distal end of the suction tube 1166. For example, FIG. 36A shows the distal end 1272 of the detection fiber 1264 aligned with a distal end of the suction tube 1166. Here, the distal end 1272 of the detection fiber 1264 is arranged on a first side of the suction tube 1166. More specifically, the distal end 1272 is arranged on the side of the suction tube 1166 in a direction of curvature of the suction tube 1166. Said differently, the suction tube 1166 illustrated herein curves downward, and the distal end 1272 of the detection fiber 1264 is arranged on the downward or bottom side of the suction tube 1166.

With continued reference to FIG. 36A, the distal end 1317 of the indicator fiber 1313 is arranged on a second side of the suction tube 1166, which is opposite the first side of the suction tube 1166. More specifically, the distal end 1317 is arranged on the side of the suction tube 1166 opposite the direction of curvature of the suction tube 1166. Said differently, the suction tube 1166 illustrated herein curves downward, and the distal end 1317 of the indicator fiber 1313 is arranged on the upward or top side of the suction tube 1166.

Referring to FIG. 36B, another implementation of the suction tool 1156 is shown. In this implementation, the detection fiber 1264 and the indicator fiber 1313 are arranged on the same side of the suction tube 1166. In the illustrated implementation, the indicator fiber 1313 is disposed on top of the detection fiber 1264. Alternatively, the first and second channels 1314, 1315 may be disposed adjacent to each other and on the same side of the suction tube 1166 such that the indicator fiber 1313 is disposed next to the detection fiber 1264.

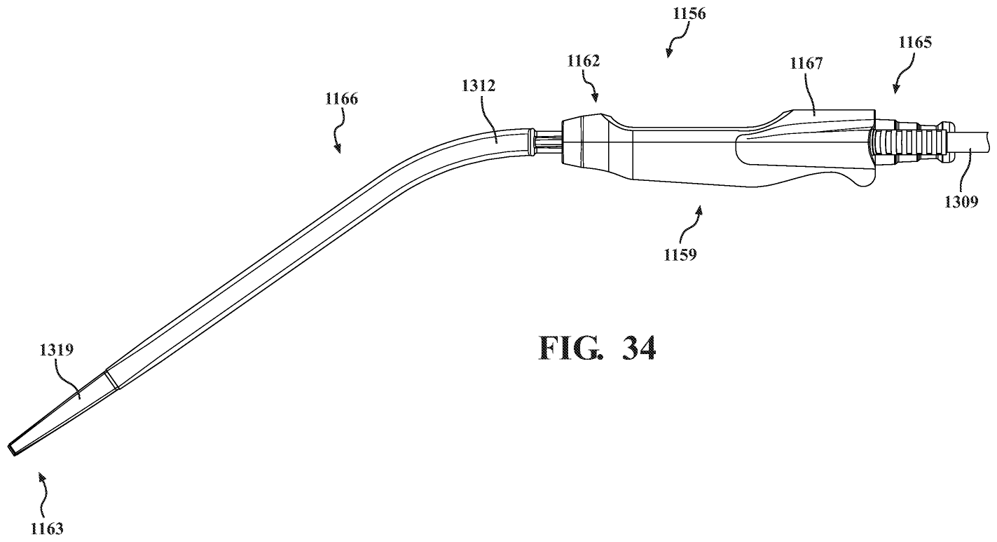

Referring to FIGS. 34-37, and 41-43, the suction tool 1156 may further comprise a sleeve surrounding a portion of the suction tube 1166, the detection fiber 1264, and the indicator fiber 1313. For specifically and in the implementation shown here, the suction tool 1156 may comprise a proximal sleeve 1312 and a distal sleeve 1319. The proximal sleeve 1312 is arranged on a portion of the outer surface 1316 of the suction tube 1166 that is proximal of the distal end of the suction tube 1166. Similarly, the distal sleeve 1319 is arranged on the outer surface 1316 of the suction tube 1166 and at the distal end of the suction tube 1166. Each of the proximal sleeve 1312 and the distal sleeve 1319 may comprise a material that is responsive to heat to change shape and/or size. For example, the proximal sleeve 1312 and the distal sleeve 1319 may comprise heat shrink tubing. As a consequence of the sleeves 1312, 1319 over the channels 1314, 1315 the optical fibers 1264, 1313 accommodated by the outer channels 1314, 1315 are held against the suction tube 1166 by the sleeves 1312, 1319. In this way, the optical fibers 1264, 1313 are secured between the suction tube 1166 and the sleeves 1312, 1319. In such configurations, the suction tool 1156 may include a suction line with the suction line tethered to the connector line 1309.

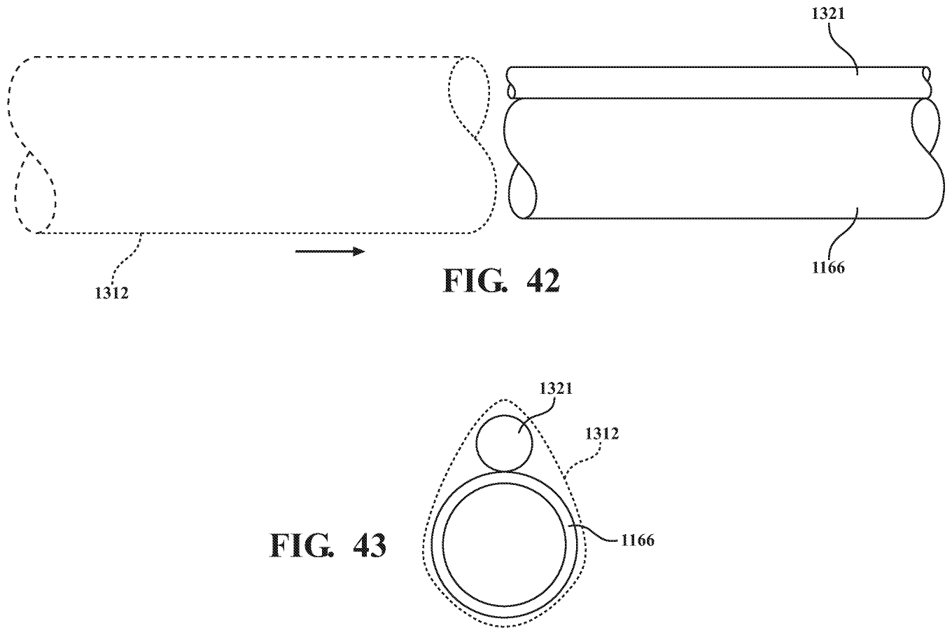

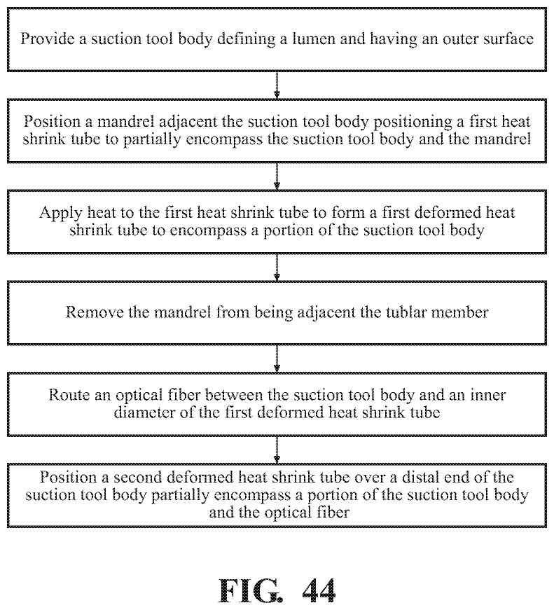

As mentioned above, the sleeves 1312, 1319 may comprise a heat shrink material that has an unrecovered state in which the size of the sleeve 1312, 1319 is enlarged, and a recovered state in which the size of the sleeve 1312, 1319 is reduced. In FIG. 42, a portion of the suction tube 1166 and a mandrel 1321 are shown with the proximal sleeve 1312 in the unrecovered state and spaced therefrom. To form the tube portion of the suction tool 1156, the mandrel 1321 is placed in the outer channel 1314 of the suction tube 1166 and the sleeve 1312 is placed over the suction tube 1166 and the mandrel 1321. Heat is applied to the sleeve 1312 to transition the sleeve from the unrecovered state, shown in FIG. 42, to the recovered state, shown in FIG. 43. Once the sleeve 1312 is in the recovered state the mandrel 1321 is removed from the sleeve 1312 the appropriate optical fiber 1264, 1313 is inserted into the channel 1314. Using the mandrel 1321 when heating the sleeve 1312 prevents damage to the optical fiber from the buildup of excess heat. The distal sleeve 1319 may be applied in a similar manner. Alternatively, the distal sleeve 1319 may be formed separately and applied using an adhesive that further facilitates retaining the optical fibers 1264, 1313 in the respective outer channels 1314, 1315.

As mentioned above, the indicator 1298 may be a visual indicator that emits light in response to the intensity of the single band associated with PPIX and based on the signal of collected light with the effect of ambient light removed being above a predetermined threshold. The indicator 1298 is coupled to the suction tube 1166 adjacent to the distal end to increase the visibility of the indicator 1298. To this end, the distal sleeve 1319 that surrounds the indicator 1298 and the indicator fiber 1313 may comprise a material that at least partially permits the transmission of visible light. The distal sleeve 1319 may be a clear or mostly clear material capable of protecting the distal end of the suction tube 1166 and holding the optical fibers 1264, 1313 to the suction tube 1166. Because the distal sleeve 1319 permits the transmission of visible light the surgeon is able to see when the indicator 1298 is illuminated and not illuminated during use.

In contrast to the distal sleeve, 1319, the proximal sleeve 1312 may comprise a material that substantially blocks the transmission of visible light. The proximal sleeve 1312 may be an opaque or mostly opaque material capable of protecting the distal end of the suction tube 1166 and holding the optical fibers 1264, 1313 to the suction tube 1166. By blocking or substantially blocking the transmission of light the proximal sleeve 1312 shields the optical fibers 1264, 1313 from any ambient light. While the transmission of light along the optical fibers 1264, 1313 is very efficient, a very small amount of light may be able to penetrate the outer surface of the optical fibers 1264, 1313. By surrounding the optical fibers 1264, 1313 with the opaque sleeve 1312, the contrast between the on and off light signals transmitted through the optical fibers 1264, 1313 is enhanced. Said differently, the difference between the light signal's “on” state and the light signal's “off” state (i.e., no light being transmitted) is more easily discernable by the controller as well as the surgeon viewing the indicator.

A method of forming a hand-held surgical probe including an optical fiber is also contemplated. The method includes providing a suction tool body defining a lumen and having an outer surface. The suction tool body may be the suction tool 156″″ described elsewhere. The method may further include positioning a mandrel 1321 adjacent the suction tool body. The method may include positioning a first heat shrink tube (such as the preformed version of 312) to partially encompass the suction tool body and the mandrel 1321. The method may include applying heat to the first heat shrink tube to form a first deformed heat shrink tube 312 to encompass a portion of the suction tool body. The method removing the mandrel 1321 from being adjacent the suction tool body. The method may include routing an optical fiber 264′″, 1313 between the suction tool body and an inner diameter of the first deformed heat shrink tube 312. The method may also include positioning a second deformed heat shrink tube 1319 over a distal end of the suction tool body partially encompass a portion of the suction tool body and the optical fiber 312, 1313.

The method may also include further comprising pre-forming the second heat shrink tube to form the second deformed heat shrink tube before positioning the second heat shrink tube over the distal end of the suction tool body. The step of pre-forming comprises applying heat to the second heat shrink tube while the second heat shrink tube is positioned about a second mandrel having a shape matching a shape of a distal end portion of the suction tool body.

As described above, the second deformed heat shrink tube is formed from a transparent material and the first heat shrink tube is formed from an opaque material. This allows light emitted from the indicator fiber 1313 to shine radially through the second deformed heat seat shrink tube near a proximal portion of the suction tool.

The optical fiber may have a melting point that is lower than the melting point of first heat shrink tube and/or the second heat shrink tube. For example, the optical fiber may have a melting point lower than 70 or 80 degrees Celsius, whereas the opaque heat shrink and/or transparent heat shrink might have a melting point about 130 or 140 degrees Celsius. The melting point of the optical fiber is at least 50 degrees Celsius lower than the melting point of the first heat shrink and/or second heat shrink.

The inventors recognized the unique formation process would allow the advantages of utilization of very thin-wall heat shrink material without causing damaging the one or more optical fibers. The utilization of such heat shrink material near the distal portion of the suction tool is advantageous as the thin-walled nature of this material prevents obstruction of the surgeon's line of sight. In some instances, a thickness of the transparent heat shrink tube is less than the thickness of the opaque heat shrink tube. The thickness of the transparent heat shrink tube may be at least 75% less than the thickness of the opaque heat shrink tube. In certain instances, the thickness of the transparent heat shrink tube is less than 2%, less than 1, or less than 0.5% of an outer diameter of the distal end of the suction tool body.

The method may include securing the second deformed heat shrink tube to the suction tool body using an adhesive. The adhesive may be UV curable.

The method may include positioning the optical fiber in a groove formed in an outer diameter of the suction tool body. The method may include a step of positioning the second deformed heat shrink tube comprises positioning the second deformed heat shrink tube such that the second deformed heat shrink tube partially surrounds the groove.

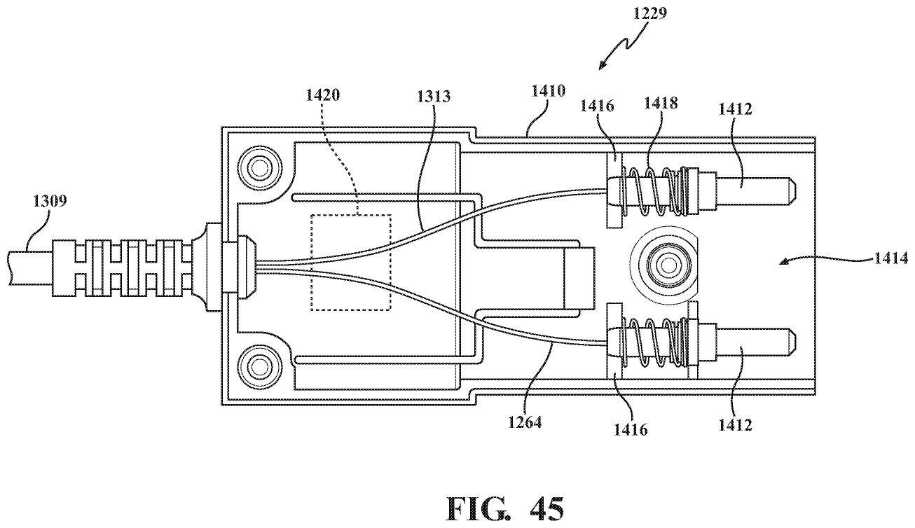

Turning now to FIG. 45, an exemplary optical connector 1229 is shown. The optical connector 1229 couples and connects the connector line 1309 to the optical system, described in greater detail above. The optical connector 1229 comprises an outer shell 1410, shown here with half of the outer shell 1410 removed. One side of the optical connector 1229 receives the connector line 1309, which includes the detection fiber 1264 and the indicator fiber 1313, and the opposite side of the optical connector 1229 engages the optical system within a surgical console, in one example. Here, the optical connector 1229 is free from an electrical terminal and, as such, the suction tool 1156 and indicator 1298 cannot conduct electricity into the patient's body during a neurosurgical procedure. The detection fiber 1264 and the indicator fiber 1313 are each terminated with a ferrule 1412 that surrounds the optical fibers and facilitates engagement with the components in the optical system. Typically, a ferrule 1412 is crimped, swaged, or bonded onto the proximal most end of each of the optical fibers before being secured in the outer shell 1410.

Each ferrule 1412 is supported in the interior 1414 of the optical connector 1229 and spaced from each other in a radial direction. The ends of each ferrule 1412 are generally aligned with one another in an axial direction. To this end, the outer shell 1410 comprises a socket 1416 for each ferrule 1412 to axially support the ferrules 1412 in the interior 1414. In the implementation shown here, the optical connector 1229 further comprises a spring 1418 disposed around one of the ferrules 1412 between the ferrule 1412 and one of the sockets 1416 outer shell 1410. The spring 1418 permits some axial and radial movement, and therefore misalignment, of the ferrule 1412 and optical fiber. The movement of the ferrule 1412 and optical fiber permitted by the spring 1418 allows the ferrule 1412 to shift slightly within the interior 1414 of the optical connector 1229 when a healthcare professional is plugging the optical connector 1229 into the surgical console. Because the ferrule 1412 and optical fiber can move or shift slightly, the outer shell 1410 can be manufactured without high precision tolerances that might otherwise be necessary to prevent a misaligned ferrule from damaging the optical system. Said differently, if the ferrules were rigidly held in a position that was slightly misaligned with the optical system, repeated connection and disconnection of the optical connector 1229 and the console could result in damage to the optical system of the console. By allowing one of the ferrules 1412 to float within the interior 1414 of the optical connector 1229, rigid misalignment of the ferrules 1412 is avoided.

While the optical connector 1229 shown here is free from an electrical terminal, the optical connecter 1229 may comprise an RFID tag 1420 capable of being read by the console. The RFID tag 1420 having a non-transitory memory may be disposed in the interior of the optical connector 1229 and coupled to the outer shell 1410. The RFID tag 1420 may contain data and other information or characteristics stored on the memory about the suction tool 1156 that may pertain to the optical fibers 1264, 1313. For example, the RFID tag 1420 may contain properties of the detection fiber 1264 such as the light transmission characteristics or peak efficiency that can be used to adjust a detection algorithm of the console. In another example, the RFID tag 1420 may include properties that could be used to adjust the detection algorithm such as length or diameter of the optical fibers. The adjustments to the detection algorithm may include increasing or decreasing the strength of the excitation light that is provided to the detection fiber 1264 to account for reduced output at the distal end 1272 of the detection fiber 1264. The RFID tag 1420 may also include usage data such as the number of times the suction tool 1156 has been connected and to how many different consoles. The usage data may also include how long the suction tool 1156 has been in use. Further still, the RFID tag 1420 could include manufacturing data such as a serial number, a manufacturing date, and an expiration date that could be used by the console to analyze usage trends and prevent expired components from being used.

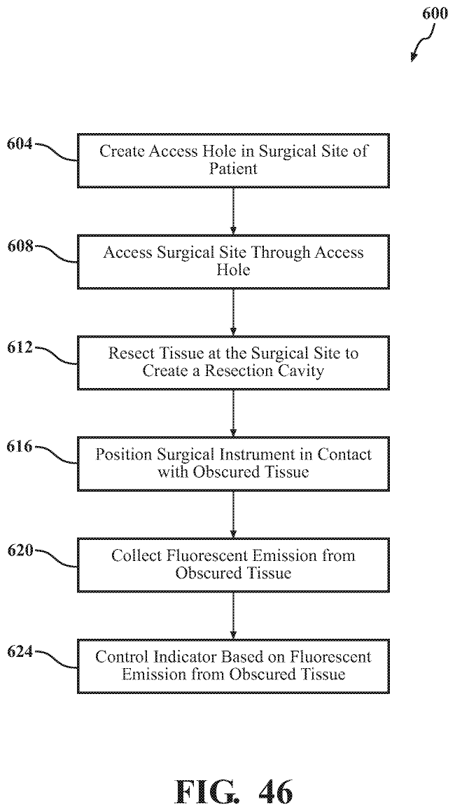

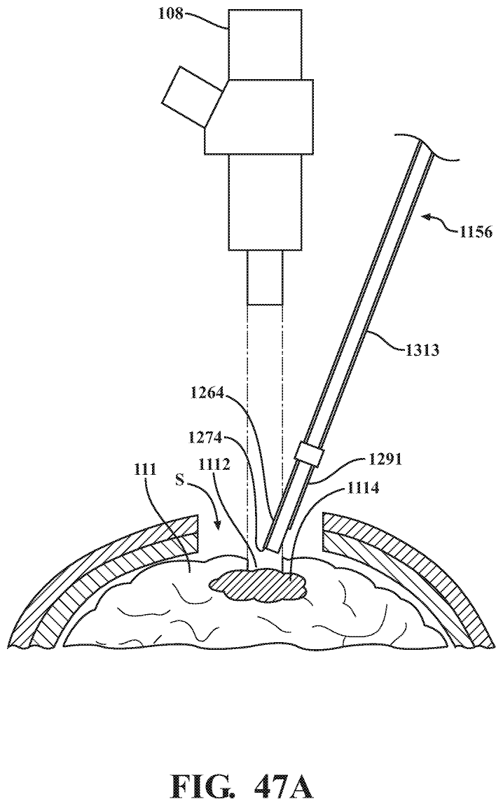

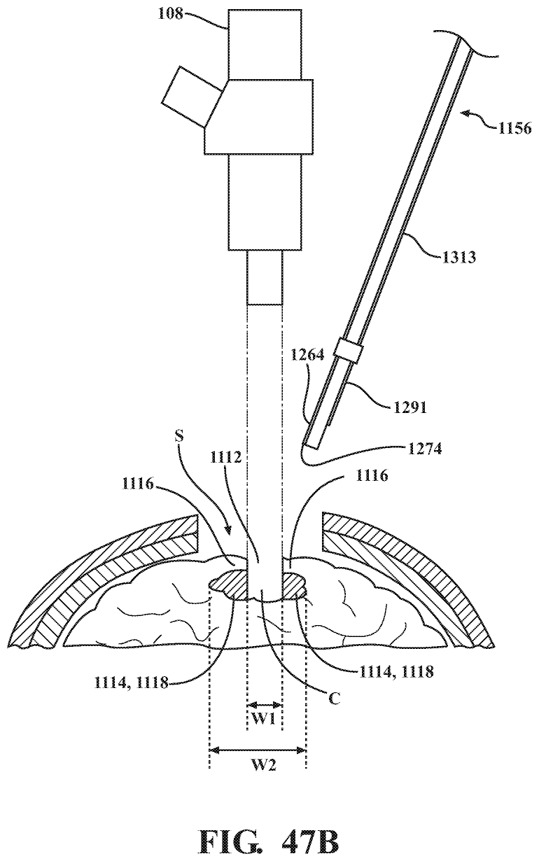

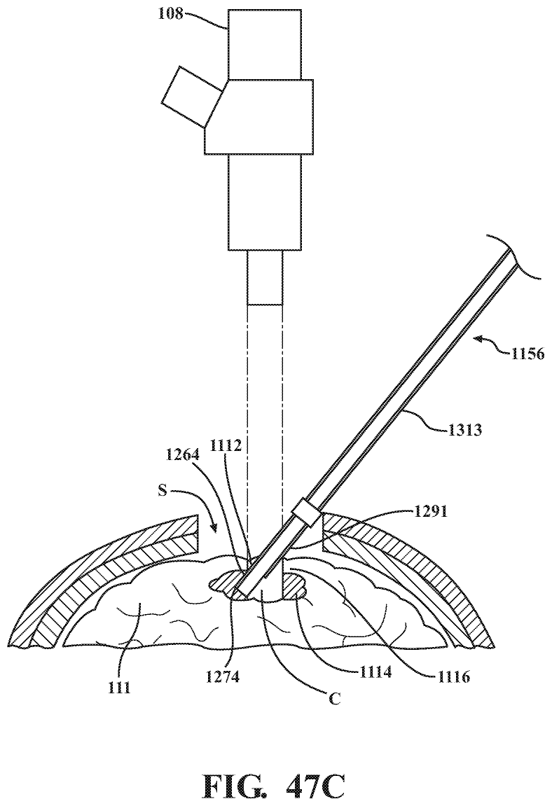

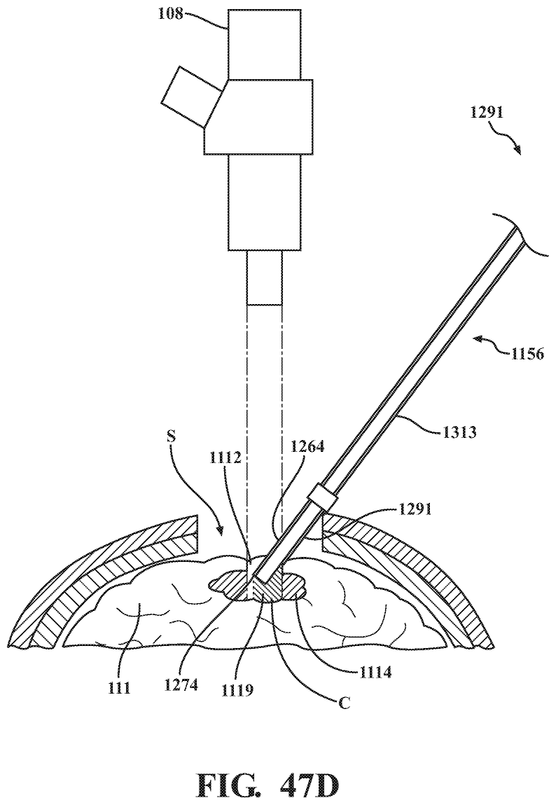

Referring to FIGS. 46-47D, a flow diagram depicting a method 600 of assessing tumor margins of obscured tissue is shown in FIG. 46, and illustrations of various steps of the method 600 are shown in FIGS. 47A-47D. The method 600 is carried out on a patient who was administered a fluorescing agent, such as 5-ALA. The method 600 is described herein, and shown in the figures, as being focused on a surgical site S which is surrounded by the brain tissue 111 of the patient, but the method 600 may be carried out on other tissues of the patient as well.

In FIG. 47A, a portion of a head of the patient is illustrated. Prior to the method 600, hard and soft tissues covering the brain tissue 111 has been removed to provide access to the brain tissue 111. If the surgical site S is within other, non-brain tissues of the patient, other tissues may be similarly removed to provide access to the relevant tissue.

At step 604, an access hole 1112 may be created through the brain tissue 111. The access hole 1112 extends to the surgical site S which, in this case, is tumorous brain tissue 1114. After the access hole 1112 is created, the surgical site S can be reached. At step 608, the surgical site S may be accessed through the access hole 1112. In the illustrated implementation, the surgical site S is accessed by the suction tool 1156 that includes the detection fiber 1264 and optionally, the indicator portion 1291 (see FIG. 36A).

At step 612, a surgical instrument may be used to resect tissue at the surgical site S to create a resection cavity C. In the implementation illustrated in FIG. 47B, the surgical instrument is the suction tool 1156 and the resected tissue includes the tumorous brain tissue 1114. The cavity C may be created by resecting tissue which is illuminated by an illumination source, such as the surgical microscope 108, and caused to fluoresce by the illumination source, such as the light emitted by the microscope 108. As noted above, the patient has been administered a fluorescing agent, such as 5-ALA, and the surgical microscope 108 is configured for fluorescence microscopy, for example, to detect PPIX/ICG. Thus, in the illustrated implementation, the tumorous brain tissue 1114 is illuminated by the light from the microscope 108 and fluoresces in response to being excited by the light.

As shown in FIG. 47B and described above, the surgical microscope 108 has a field of view, the relevant area of which is represented by the area between the dotted lines extending from the microscope 108 and towards the surgical site S. This portion of the field of view is relevant because it is the part of the field of view which includes the surgical site and extends through the access hole 1112. In other words, the portion of the field of view of the microscope 108 shown in FIGS. 47B-47D shows a limitation of using the microscope 108, such as by illuminating the tumorous brain tissue 1114. This limitation arises when attempting to access the surgical site through the access hole 1112, because the access hole 1112 is narrower than the surgical site S (e.g., the tumorous brain tissue 1114) being accessed through the hole 1112. For example, in the illustrated implementation, the access hole 1112 has a first width W1 and at least a portion of the tumorous tissue 1114 spans a second width W2, and the second width W2 is larger than the first width W1. This difference in width results in a tissue shelf 1116, which obscures the field of view of the surgical microscope 108 and obscures at least a portion of the tissue at the surgical site S. The tissue at the surgical site S that cannot be seen/illuminated by the microscope 108 or light source due to the tissue shelf 1116 is hereby referred to as obscured tissue 1118. Since the obscured tissue 1118 cannot be illuminated by the surgical microscope 108 due to the tissue shelf 1116, the obscured tissue 1118 must be illuminated by the surgical instrument being inserted through the access hole 1112, such as by the detection fiber 1264 of the suction tool 1156.

In FIG. 47B, the cavity C has been created by resecting tissue at the surgical site S until at least a portion of the cavity C is as wide as the access hole 1112. The tissue, such as the tumorous brain tissue 1114, may have been illuminated by the surgical microscope 108 while the tissue was resected. Alternatively, the tissue may have been illuminated by the surgical instrument, such as by the detection fiber 1264 of the suction tool 1156. Once the tissue at the surgical site S has been resected to create the cavity C shown in FIG. 47B, the healthcare professional must rely on the surgical instrument to provide the excitation light to the remainder of the surgical site S. The portion of the resection cavity C which is within the field of view of the surgical microscope 108 may be considered a first resection volume, while the remainder of the resection cavity C (i.e. the portion that is outside the field of view of the surgical microscope 108) may be considered a second resection volume.

At step 616 and as shown in FIG. 47C, the surgical instrument (e.g. the suction tool 1156) is positioned through the access hole 1112 and within the resection cavity C such that the instrument is in contact with the obscured tissue 1118. In other words, such that the instrument is in contact with the second resection volume. In the illustrated implementation, the suction tool 1156 is positioned through the access hole 1112 such that the detection fiber 1264 contacts the obscured tissue 1118. At step 620, the surgical instrument both provides the excitation light to the obscured tissue 1118 and collects any resultant fluorescent emission from the obscured tissue 1118. Again, in the illustrated implementation, the surgical instrument is the suction tool 1156 and the detection fiber 1264 both provides the excitation light and collects the fluorescent emission from the obscured tissue 1118. Finally, at step 624, the indicator of the tissue detection system is controlled by the tissue detection system based on the fluorescent emissions from the obscured tissue 1118. Continuing with the illustration implementation, the distal end 1317 of the indicator fiber 1313 of the suction tool 1156 is controlled to emit light when the detection fiber 1264 collects fluorescent emissions from the obscured tissue 1118.

The method 600 may further include resecting the tumorous tissue 1114 based on the fluorescence collected at step 620 and/or the state of the indicator at step 624. More specifically, the method 600 may include another step after step 624 that includes resecting obscured tissue based on the fluorescence collected at step 620 and/or the state of the indicator at step 624, and the method 600 may return to step 616 after this step in a loop-wise manner. In such an implementation, the method 600 may include resecting a first portion of the obscured tissue 1118 with the surgical instrument after collecting fluorescent emissions from the same tissue and activating the indicator in response. Once the first portion of the obscured tissue 1118 has been resected, the instrument may not detect fluorescent emissions due to a portion of the tumorous tissue 1114 being removed. Since other portions of the obscured tissue 1118 maystill include tumorous tissue 1114, the surgical instrument may be repositioned within the resection cavity C until fluorescent emissions are detected, such as by the detection fiber 1264. Since the detection fiber 1264 mayneed to be close to/in contact with tumorous tissue 1114 to detect fluorescence, the instrument (e.g. the suction tool 1156) may need to repositioned within the cavity C to be near this tissue 1114. This may be difficult when accessing the surgical site S through the access hole 1112, because the access hole 1112 maylimit the degree to which the instrument may be pivoted within the resection cavity C without abutting a side of the access hole 1112. For example, in FIG. 47C, the suction tool 1156 is illustrated with the maximum angle of tilt possible without urging the instrument against a side of the access cavity 1112.

To overcome the challenge of accessing the surgical site S through the access hole 1112, the method 600 may include positioning the surgical instrument in contact with a second portion of the obscured tissue 1118 by displacing other tissue at the surgical site without resecting the other tissue. Continuing the above example, the suction tool 1156 may be tilted even further than shown in the figures to cause the detection fiber 1264 to contact the tumorous tissue 1114 illustrated between the surgical instrument and the tissue shelf 1116. In FIG. 47C, the tool 1156 abuts a top of the tissue shelf 1116 on one side of the access hole 1112 and a bottom of the tissue shelf 1116 on the opposite side of the access hole 1112. As the tool 1156 is pivoted further downward, a portion of the tissue shelf 1116 may be temporarily displaced by the suction tool 1156 as the detection fiber 1264 is urged upwards towards tumorous tissue 1114. By displacing the tissue shelf 1116, the portion of the tumorous tissue 1114 residing near the bottom of the tissue shelf 1116 may be analyzed and resected by the surgical instrument.

The method 600 also overcomes the challenges presented by using the surgical microscope 108 to identify tumorous tissue at surgical site which are accessed through an access hole/channel. More specifically, accessing the surgical site through the access hole provides the benefit of a reduction in the amount of healthy tissue that must be removed to reach the surgical site, but also presents the challenge of limiting the field of view of the surgical microscope 108 and thereby reducing the efficacy of viewing the surgical site with the microscope 108 since it may not be able to view the tumorous tissue as described above and shown in the figures. In addition, the excitation light provided by the microscope and/or other source (overhead light, etc.) may not be able to reach the tumorous tissue. As provided herein, the method 600 overcomes these challenges by having a surgical instrument, which is insertable through the access hole, provide the excitation light to tissue at the surgical site which is obscured from the field of view of the surgical microscope 108. Moreover, the systems and method 600 provided herein overcome the challenge of detecting fluorescent emissions from tumorous tissue which may be obscured from view of the healthcare professional by a tissue shelf (e.g. the tissue shelf 1116). Instead of relying on the field of view of the surgical microscope 108 and/or the healthcare professional, the surgical instrument insertable through the access hole includes a detection fiber capable of collecting the fluorescent emissions from the tumorous tissue. Thus, as in the method 600, the surgical instrument may be inserted through the access hole and adjacent to/in contact with the tumorous tissue to detect the fluorescent emissions where the surgical microscope 108 and/or healthcare professional would otherwise struggle to do the same.

Additionally, the indicator may include a reposition indicator configured to indicate to the healthcare professional that the surgical microscope 108 should be repositioned. The reposition indicator may indicate that the field of view of the surgical microscope 108 should be changed to cause at least a portion of the obscured tissue to be within said field of view. For example, the reposition indicator may be used to permit the maximum amount of tumorous tissue to be resected without relying on the surgical instrument itself, such as without relying on the detection fiber 1264 of the suction tool 1156 and/or excitation light provided by the same. As shown in FIG. 47D, inserting a surgical instrument, such as the suction tool 1156, through the access hole 1112 may also obscure at least a portion of the tissue at the surgical site S from view of the surgical microscope 108. Herein, any tissue obscured by the instrument in this way is referred to as shadowed tissue 1119. The shadowed tissue 1119 should not be confused with the obscured tissue 1118—the shadowed tissue 1119 is that which is obscured by the instrument, while the obscured tissue 1118 is that which is obscured by other tissue, such as the tissue shelf 1116. Since the shadowed tissue 1119 may be obscured from view of the surgical microscope 108, the method 600 may include positioning the surgical instrument in contact with the shadowed tissue 1119, collecting fluorescent emission from the shadowed tissue with at least one optical fiber of the surgical instrument, such as the detection fiber 1264 of the suction tool 1156, and controlling the indicator of the tissue detection system (e.g. the indicator fiber 1313) based on the fluorescent emissions from the shadowed tissue 1119 as collected by the at least one optical fiber of the surgical instrument. In some cases, the surgical instrument may be operated in an observation mode (described below) and moved a small distance away from the shadowed tissue. Once the instrument is in this position, the excitation light (e.g., blue light) may be projected onto the shadowed tissue to cause the shadowed tissue to fluoresce. The indicator (e.g., the indicator fiber 1313) may then be controlled in response to fluorescent emission from the shadowed tissue. For example, the indicator may be disabled to allow the surgeon to observe the fluorescent emissions from the shadowed tissue without interference from light emitted by the indicator. Additionally or alternatively, the indicator may be activated based on fluorescent emissions collected from the shadowed tissue.

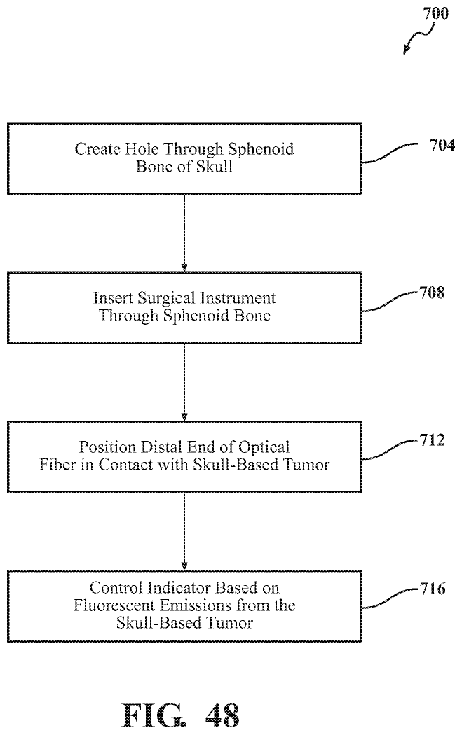

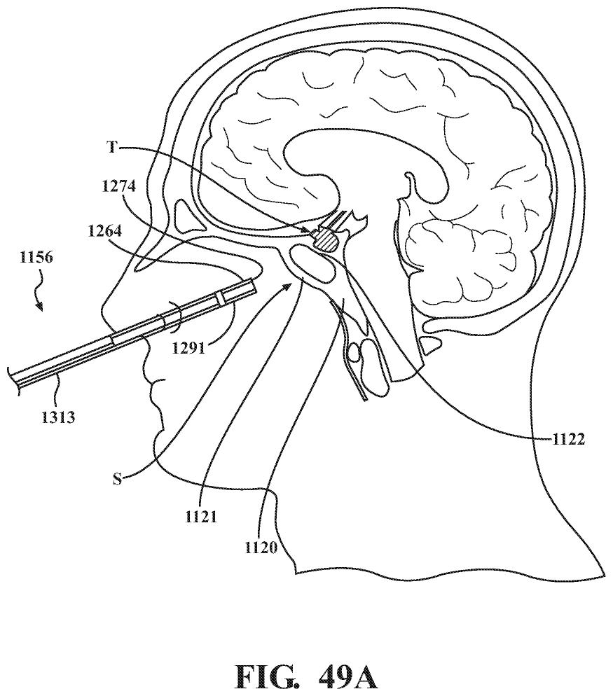

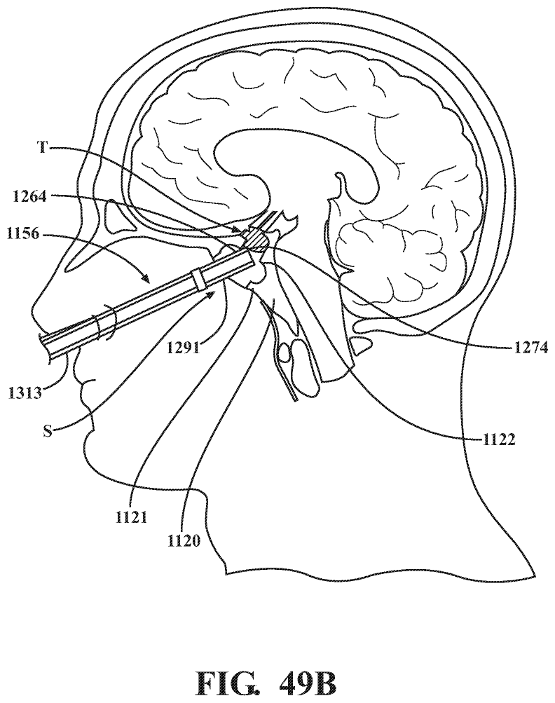

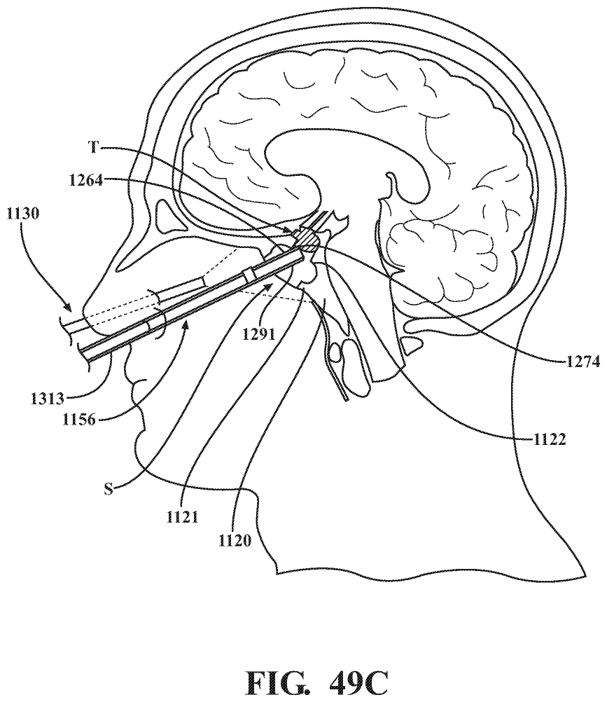

Referring to FIG. 48-49C, a flow diagram depicting a method 700 of assessing a skull-base tumor is shown in FIG. 48, and illustrations of various steps of the method 700 are shown in FIGS. 49A-49C. The method 700 is carried out on a patient who was administered a fluorescing agent, such a 5-ALA. The method 700 is described herein, and shown in the figures, as being focused on a pituitary tumor and a surgical site S which is behind a sphenoid bone of the patient, but the method 700 may be carried out on other types of skull-base tumors as well.

In FIG. 49A, a partial cross section of the head of the patient is shown. As described in more detail below, the skull-base tumor is accessed through the nose and sinus of the patient (i.e. trans-nasally). In the figures, the nose and sinus structures are not shown in detail, but the method 700 should be understood as taking place after the surgical instrument has been inserted therethrough. The method 700 is also carried out using a tissue detection system which includes an optical system, such as the tissue detection system 116 and the optical system 215, and a surgical instrument which includes at least one optical fiber extending to a distal end of the instrument for transmitting excitation light and receiving fluorescent emissions, such as the suction tool 1156. Further, since the illustrated implementation is focused on a pituitary tumor, the cross-sectional view of the head of the patient only includes tissues of the head which are most pertinent to operations involving this kind of tumor.

Starting with step 704 of the method 700, a hole is created through a sphenoid bone 1120 of the patient to allow the surgical instrument to reach the skull-base tumor T. In the illustrated implementation, step 704 includes create holes through both a proximal wall 1121 and a distal wall 1122 of the sphenoid bone 1120. Subsequently, at step 708, the surgical instrument, such as the suction tool 1156, is inserted trans-nasally through the proximal and distal walls 1121, 1122 of the sphenoid bone 1120. As shown in FIG. 49B, at step 712, a distal end of the optical fiber of the instrument is positioned in contact with the skull-base tumor. In the illustrated implementation, step 712 includes positioning the distal end 1274 of the detection fiber 1264 of the suction tool 1156 in contact with the pituitary tumor T. At step 716, the indicator of the tissue detection system is controlled based on the fluorescent emissions from the skull-base tumor. Continuing with the illustrated implementation, the indicator may be the indicator fiber 1313 of the suction tool 1156.

As shown in FIG. 49C, the method 700 may include inserting an 1130 endoscope trans-nasally, such as through the proximal and distal wall 1121, 1122 of the sphenoid bone 1120, so that a field of view of the 1330 endoscope includes at least a portion of the skull-base tumor T of the patient. In such an implementation, since multiple tools would be inserted trans-nasally during this implementation of the method 700, the surgical instrument may be inserted through a first nostril of the patient and the endoscope may be inserted through a second nostril of the patient. As such, instead of an indicator which is coupled to the instrument like the indicator fiber 1313, the endoscope may also be coupled to at least one display 120 and the display 120 may include the indicator. For example, the display 120 may be configured to display a graphical user interface, and the graphical user interface may include the indicator. The display 120 may also include the reposition indicator which directs the healthcare professional to change a field of view of the endoscope.

Some of the steps of the methods 600, 700 are described as including positioning the surgical instrument in contact with tissue to detect fluorescence, but it should be understood that the surgical instrument may also be positioned different relative to the tissue to detect fluorescence, such as adjacent to these tissues. Further, the surgical instrument used to carry out either of the methods 600, 700 may include a bend anywhere between proximal and distal ends of the instrument to reduce the amount of tissue obscured by the surgical instrument and to increase the visibility of the distal end of the instrument. For example, see the bend in the suction tool 156 shown in FIG. 2. The surgical instrument may also be an ultrasonic resection tool or bipolar forceps, such as the ultrasonic surgical system 118 or bipolar forceps 160.

In either of the methods 600, 700, the surgical instrument may be the suction tool 1156 configured to switch between modes based on whether fluorescence emissions are detected by the detection fiber 1264. For example, the suction tool 1156 may be operable in an observation mode and a resection mode. In the observation mode, the tool 1156 is configured to detect fluorescent emission from nearby tissue, such as the obscured tissue 1118, without applying a suction force to the tissue. In the resection mode, on the other hand, the tool 1156 is configured to detect fluorescent emission from nearby tissue while applying the suction force to the same tissue, such as the obscured tissue 1118. This implementation provides the benefit of automatically controlling the effecting portion of the surgical instrument based on fluorescence detected by the same instrument (or by the endoscope in some implementation). Further, the observation mode may be used to assist the healthcare professional with identifying fluorescent emissions from nearby tissue. More specifically, the healthcare professional may increase the amount of nearby tissue being illuminated by excitation light from the detection fiber 1264 by moving the surgical instrument away from the nearby tissue. For example, the excitation light from the detection fiber 1264 may be projected as a cone of excitation light which has an increasing radius/width as the light projects further away from the detection fiber 1264. By moving the detection fiber 1264 away from the nearby tissue, the cone of excitation light illuminates a larger area of nearby tissue and causes the larger area of nearby tissue to fluoresce in response. Further, the suction tool 1156 may be operable in a manual detection mode in which the indicator of the surgical suction tool is disabled and does not respond to detections of fluorescent emission from tissue. The suction tool 1156 may be in the manual detection mode while also in one of the observation and resection modes. For example, the healthcare professional may put the suction tool 1156 into manual detection mode and observation mode such that the tool 1156 emits excitation light but does not otherwise apply suction force or activate the indicator. The manual detection mode may be useful in cases where the healthcare professional desires to observe the fluorescent emissions with their own vision and without aid from the indicator.

It should be appreciated that the devices, methods, and systems described throughout may be used for tissue types other than brain tissue, such as breast tissue. In these circumstances, the systems may be referred to as surgical systems or surgical methods, rather than neurosurgical systems and methods.

While the construction of particular devices and systems are contemplated throughout, it should be appreciated that the fibers, heat shrink sleeve(s) may be used in conjunction with other device types, such as scalpels, electrosurgical devices, such as electrosurgical pencils, forceps, surgical burring devices, endoscopes, stereotactic frames, pointers, stylets, and the like.

### Clauses

Clause 1—An ultrasonic surgical system comprising: an ultrasonic handpiece assembly configured to remove brain tissue, the ultrasonic handpiece assembly; a sample element coupled to the ultrasonic handpiece assembly and including at least one fiber configured to collect a fluorescent light emitted from the brain tissue; an indicator coupled to the ultrasonic handpiece assembly configured to selectively emit light; a controller configured to: detect a type of brain tissue based on the fluorescent light; activate the indicator based on the detected type of brain tissue; and control the ultrasonic handpiece assembly based on the detected type of brain tissue.
Clause 2—The ultrasonic surgical system of clause 1, wherein the at least one fiber is coupled to an excitation source, the at least one fiber configured to illuminate an excitation light from the excitation source to induce the fluorescent light and collect the fluorescent light emitted from the brain tissue.
Clause 3—The ultrasonic surgical system of clause 2, further comprising an optical system coupled to the controller and the sample element, the optical system including the excitation source and an optical detection system configured to convert the fluorescent light into an electrical signal wherein the controller detects the type of brain tissue from the electrical signal.
Clause 4—The ultrasonic surgical system of clause 3, wherein: the excitation source is further defined as a first excitation source, the fluorescent light is further defined as a first fluorescent light, and the electrical signal is further defined as a first electrical signal; the optical system further including a second excitation source; the at least one fiber configured to illuminate a second excitation light from the second excitation source to induce a second fluorescent light emitted from the brain tissue and collect the second fluorescent light; the optical detection system configured to convert the second fluorescent light into a second electrical signal; and the controller configured to determine a second type of brain tissue from the second electrical signal.
Clause 5—The ultrasonic surgical system of clause 3, wherein the controller is configured to detect the type of brain tissue based on an algorithm.
Clause 6—The ultrasonic surgical system of clause 5, wherein the algorithm includes a calibration routine to be performed with respect to healthy tissue or a baseline parameter.
Clause 7—The ultrasonic surgical system of clause 5, wherein the algorithm is configured to calculate a modified electrical signal by fitting a baseline polynomial curve to the electrical signal and subtract the baseline polynomial curve from the electrical signal to remove ambient light.
Clause 8—The ultrasonic surgical system of clause 7, wherein the algorithm includes fitting at least one gaussian distribution to the modified electrical signal.
Clause 9—The ultrasonic surgical system of clause 5, wherein: the controller is configured to cycle the excitation source on and off; the sample element is configured to collect ambient light when the excitation source is off and not illuminating the brain tissue with the fluorescent light; the sample element configured to collect ambient light and the fluorescent light when the excitation source is on and the sample element is illuminating the brain tissue with the fluorescent light; and wherein the algorithm includes subtracting the ambient light from the fluorescent light.
Clause 10—The ultrasonic surgical system of clause 1, wherein the ultrasonic handpiece assembly includes an ultrasonic handpiece and a sleeve, the indicator being coupled to the sleeve.
Clause 11—The ultrasonic surgical system of clause 1, further comprising an electrode configured to apply electrical stimulation to the brain tissue wherein the controller generates an alert when the electrical stimulation produces a predefined response from a patient.
Clause 12—A surgical system comprising: surgical tool configured to remove brain tissue; a sample element coupled to the surgical tool and including at least one fiber configured to collect (i) a fluorescent light emitted from the brain tissue; an indicator coupled to the surgical tool and configured to selectively emit light; a controller configured to: detect a type of brain tissue based on the fluorescent light; activate the indicator based on the detected type of brain tissue; and control the surgical tool based on the detected type of brain tissue.
Clause 13—The surgical system of clause 12 further comprising an optical system coupled to the controller and the sample element, the optical system including: an excitation source coupled to the at least one fiber, the at least one fiber configured to illuminate the brain tissue with an excitation light from the excitation source to induce the fluorescent light and collect the fluorescent light emitted from the brain tissue; and an optical detection system configured to convert the fluorescent light into an electrical signal wherein the controller detects the type of brain tissue from the electrical signal.
Clause 14—The surgical system of clause 13, wherein: the excitation source is further defined as a first excitation source, the fluorescent light is further defined as a first fluorescent light, and the electrical signal is further defined as a first electrical signal; the optical system further including a second excitation source; the at least one fiber configured to illuminate a second excitation light from the second excitation source to induce a second fluorescent light emitted from the brain tissue and collect the second fluorescent light; the optical detection system configured to convert the second fluorescent light into a second electrical signal; and the controller configured to determine a second type of brain tissue from the second electrical signal.
Clause 15—The surgical system of clause 13, wherein the controller is configured to detect the type of brain tissue based on an algorithm.
Clause 16—The surgical system of clause 15, wherein the algorithm includes a calibration routine to be performed with respect to healthy tissue or a baseline parameter.
Clause 17—The surgical system of clause 15, wherein the algorithm is configured to calculate a modified electrical signal by fitting a baseline polynomial curve to the electrical signal and subtract the baseline polynomial curve from the electrical signal to remove ambient light.
Clause 18—The surgical system of clause 17, wherein the algorithm includes fitting at least one gaussian distribution to the modified electrical signal.
Clause 19—The surgical system of clause 15, wherein the controller is configured to cycle the excitation source on and off; the sample element is configured to collect ambient light when the excitation source is off and not illuminating the brain tissue with the fluorescent light; the sample element is configured to collect ambient light and the fluorescent light when the excitation source is on and the sample element is illuminating the brain tissue with the fluorescent light; and wherein the algorithm includes subtracting the ambient light from the fluorescent light.
Clause 20—The surgical system of clause 12, wherein the surgical tool comprises bipolar forceps.
Clause 21—The surgical system of clause 12, wherein the surgical tool comprises a neuro stimulator.
Clause 22—The surgical system of clause 12, wherein the surgical tool comprises a neuro dissector.
Clause 23—The surgical system of clause 12, wherein the surgical tool comprises an ablation device.
Clause 24—The surgical system of clause 12, wherein the controller is configured to control the surgical tool by adjusting an operating parameter of the surgical tool based on the detection of the type of brain tissue.
Clause 25—The surgical system of clause 12, further comprising an electrode configured to apply electrical stimulation to the brain tissue.
Clause 26—The surgical system of clause 25, wherein the controller generates an alert when the electrical stimulation produces a predefined response from a patient.
Clause 27—A surgical suction system comprising: suction tool configured to apply suction to brain tissue; a sample element coupled to the suction tool including at least one optical fiber configured to collect a fluorescent light emitted from the brain tissue; an indicator coupled to the suction tool and configured to selectively emit light; a controller configured to: detect a type of brain tissue based on the fluorescent light; and activate the indicator on the detected type of brain tissue.
Clause 28—The surgical suction system of clause 27, wherein the at least one optical fiber is coupled to an excitation source, the at least one fiber configured to illuminate an excitation light from the excitation source to induce the fluorescent light and collect the fluorescent light emitted from the brain tissue.
Clause 29—The surgical suction system of clause 28, further comprising an optical system coupled to the controller and the sample element, the optical system including the excitation source and an optical detection system configured to convert the fluorescent light into an electrical signal wherein the controller detects the type of brain tissue from the electrical signal.
Clause 30—The surgical suction system of clause 29, wherein: the excitation source is further defined as a first excitation source, the fluorescent light is further defined as a first fluorescent light, and the electrical signal is further defined as a first electrical signal: the optical system further including a second excitation source; the at least one fiber configured to illuminate a second excitation light from the second excitation source to induce a second fluorescent light emitted from the brain tissue and collect the second fluorescent light; the optical detection system configured to convert the second fluorescent light into a second electrical signal; and the controller configured to determine a second type of brain tissue from the second electrical signal.
Clause 31—The surgical suction system of clause 29, wherein the controller is configured to detect the type of brain tissue based on an algorithm.
Clause 32—The surgical suction system of clause 31, wherein the algorithm includes a calibration routine to be performed with respect to healthy tissue or a baseline parameter.
Clause 33—The surgical suction system of clause 31, wherein the algorithm is configured to calculate a modified electrical signal by fitting a baseline polynomial curve to the electrical signal and subtract the baseline polynomial curve from the electrical signal to remove ambient light.
Clause 34—The surgical suction system of clause 33, wherein the algorithm includes fitting at least one gaussian distribution to the modified electrical signal.
Clause 35—The surgical suction system of clause 31, wherein: the controller is configured to cycle the excitation source on and off; the sample element is configured to collect ambient light when the excitation source is off and not illuminating the brain tissue with the fluorescent light; the sample element is configured to collect ambient light and the fluorescent light when the excitation source is on and the sample element is illuminating the brain tissue with the fluorescent light; and wherein the algorithm includes subtracting the ambient light from the fluorescent light.
Clause 36—The surgical suction system of clause 27, further comprising an electrode configured to apply electrical stimulation to the brain tissue, wherein the controller generates an alert when the electrical stimulation produces a predefined response from a patient.
Clause 37—The surgical suction system of clause 27, the suction tool including a handle portion and an elongated portion, the sample element being coupled to the elongated portion.
Clause 38—A method for detecting target tissue under ambient light conditions in an operating room during a surgical procedure, the method comprising: positioning, an optical fiber, in a sterile field that includes brain tissue being illuminated by ambient light; collecting, with the optical fiber, fluorescent light emitted from the brain tissue; detecting, with a controller coupled to the optical fiber, target tissue of the brain tissue based on fluorescent light emitted from the brain tissue; and activating an indicator positioned within the sterile field to produce a visual alert in response to the detection of the target tissue.
Clause 39—The method of clause 38, wherein the fluorescent light is emitted from the brain tissue in response to illuminating the brain tissue with excitation light from an excitation source coupled to the optical fiber.
Clause 40—The method of clause 39, wherein the detecting the fluorescent light emitted from the target tissue during the surgical procedure is based on an algorithm.
Clause 41—The method of clause 40, wherein the algorithm includes a calibration routine to be performed with respect to healthy tissue or a baseline parameter.
Clause 42—The method of clause 40, further comprising an optical system coupled to the optical fiber and the controller, the optical system configured to convert the fluorescent light collected from the target tissue into an electrical fluorescent signal.
Clause 43—The method of clause 42, wherein the optical system includes a spectrometer.
Clause 44—The method of clause 42, wherein the algorithm includes calculating a modified electrical fluorescent signal by fitting a baseline polynomial curve to the electrical fluorescent signal and subtracting the baseline polynomial curve from an electrical fluorescent signal to remove the ambient light.
Clause 45—The method of clause 44, wherein the algorithm includes fitting at least one gaussian distribution to the modified electrical fluorescent signal.
Clause 46—The method of clause 40, further comprising: cycling the excitation light from an excitation source on and off; collecting ambient light, with the optical fiber, when the excitation light is off and the brain tissue is not illuminated by the excitation light; and wherein the algorithm includes subtracting the ambient light from the fluorescent light.
Clause 47—The method of clause 38, wherein the optical fiber is integrated into at least one of a surgical tool and a standalone device.
Clause 48—The method of clause 38, wherein the indicator comprises a light emitting device coupled to a surgical instrument.
Clause 49—A method for detecting target tissue under ambient light conditions in an operating room using a surgical system, the surgical system comprising a working tool including at least one optical fiber, an indicator an optical system coupled to the working tool, and an excitation source coupled to the at least one optical fiber, the method for detecting target tissue comprising: positioning the working tool in a sterile field that includes brain tissue illuminated by ambient light; detecting, with the optical system, fluorescence light emitted from the target tissue during a surgical procedure with the least one optical fiber of the working tool, wherein detecting the fluorescence light includes: illuminating tissue with blue light from the excitation source with the at least one optical fiber; collecting, with the at least one optical fiber, the ambient light and the fluorescence light; generating a fluorescence signal based on the fluorescence light emitted from the target tissue with the ambient light removed; determining that the target tissue is present based on the detected fluorescence light; and activating the indicator of the working tool in response to detection of the target tissue.
Clause 50—The method for detecting target tissue of clause 49, wherein: illuminating tissue with blue light from the excitation source with the at least one optical fiber is performed over a first period of time and illuminating tissue with blue light is not performed over a second period of time; collecting the ambient light and the fluorescence light is performed over the first period of time; detecting the fluorescence light further includes: collecting, with the at least one optical fiber, the ambient light over a second period of time; generating (i) a first signal based on the ambient light and the fluorescence light collected during the first period of time, (ii) a second signal based on the ambient light collected during the second period of time; and the ambient light is removed from the fluorescence signal using an algorithm based on the first signal and the second signal.
Clause 51—The method for detecting target tissue of clause 50, wherein the algorithm includes calculating a baseline curve using a least-squares polynomial based on the difference of the first signal and the second signal.
Clause 52—The method for detecting target tissue of clause 51, wherein the algorithm includes subtracting the baseline curve from the difference of the first signal and the second signal to obtain the fluorescence signal.
Clause 53—The method for detecting target tissue of clause 52, wherein the algorithm includes calculating at least one gaussian curve for at least one spectral band of the fluorescence signal.
Clause 54—The method for detecting target tissue of clause 49, wherein the working tool is a suction handle.
Clause 55—The method for detecting target tissue of clause 49, wherein the working tool is an ablation device.
Clause 56—The method for detecting target tissue of clause 55, further comprising removing, with the working tool, the target tissue based on the indicator.
Clause 57—The method for detecting target tissue of clause 49, wherein the ambient light includes light generated by a surgical microscope and light generated by one or more surgical lamps.
Clause 58—The method for detecting target tissue of clause 49, the surgical system including a display, the method further comprising displaying the fluorescence signal at the display.
Clause 59—The method for detecting target tissue of clause 49, wherein the working tool includes at least one electrode configured to apply a stimulating current to target tissue, the method for detecting target tissue further comprising: applying electrical stimulation to the target tissue; and determining whether the electrical stimulation of the target tissue affects a patient.
Clause 60—The method for detecting target tissue of clause 59, wherein in response to the determination that the electrical stimulation affects the patient, controlling operation of the working tool in order to prevent the working tool from operating on the target tissue.
Clause 61—The method for detecting target tissue of clause 60, wherein controlling operation of the working tool includes changing an operation parameter of the working tool.
Clause 62—The method for detecting target tissue of clause 61, wherein the operation parameter includes at least one of an applied voltage, a current drawn, and power consumption.
Clause 63—The method for detecting target tissue of clause 59, wherein the indicator is further defined as a first indicator, the working tool includes a second indicator, the method for detecting target tissue further comprising activating the second indicator in response to the determination that the electrical stimulation affects the patient.
Clause 64—The method for detecting target tissue of clause 49, wherein the target tissue is further defined as a first target tissue, the method further comprising detecting, with the optical system, a second target tissue based on a second fluorescence light emitted from the second target tissue.
Clause 65—The method for detecting target tissue of clause 64, further comprising in response to detecting the second target tissue, controlling operation of the working tool in order to prevent the working tool from operating on the second target tissue.
Clause 66—The method for detecting target tissue of clause 64, wherein the second target tissue corresponds to a blood vessel.
Clause 67—The method for detecting target tissue of clause 49, wherein detecting the fluorescence light is performed in less than one second.
Clause 68—A method for detecting and removing target tissue under ambient light conditions in an operating room using a surgical system, the surgical system comprising a first working tool, a second working tool, an attachment including at least one optical fiber, an indicator, an optical system, and an excitation source coupled to the at least one optical fiber, the method for detecting target tissue comprising: coupling the attachment to at least one of the first working tool and the second working tool; detecting, with the optical system, a target tissue during a surgical procedure based on fluorescence light emitted from the target tissue; activating the indicator of the attachment, while at least one of the first working tool and the second working tool is in a sterile field, in response to detection of the target tissue; viewing the indicator of the attachment while at least one of the first working tool and at least one of the second working tool is within the sterile field; performing a first surgical operation at a surgical site while the first working tool is in a first hand of the operator; and performing a second surgical operation at the surgical site while the second working tool is in a second hand of the operator in response to the indicator while maintaining the first working tool in the first hand.
Clause 69—The method for detecting and removing target tissue of clause 68, wherein the first working tool corresponds to a suction cannula, the first surgical operation includes suctioning fluid from the surgical site.
Clause 70—The method for detecting and removing target tissue of clause 68, wherein the second working tool corresponds to bipolar forceps.
Clause 71—A method for detecting and removing target tissue under ambient or microscope light conditions in an operating room using a surgical system, the surgical system comprising a suction cannula including at least one optical fiber, an indicator, a working tool, an optical system, and an excitation source coupled to the at least one optical fiber, comprising: positioning the working tool and the suction cannula in a sterile field that includes brain tissue illuminated by ambient light; detecting, with the optical system, a target tissue during a surgical procedure based on fluorescence light emitted from the target tissue; activating the indicator of the suction cannula, while the suction cannula is in a sterile field, in response to detection of the target tissue; viewing the indicator of the suction cannula while the suction cannula is within the sterile field; suctioning fluid from a surgical site with the suction cannula while the suction cannula is in a first hand of an operator; and operating on the target tissue, with the working tool in a second hand of the operator, in response to the indicator while maintaining the suction cannula in the first hand of the operator.
Clause 72—A method for detecting target tissue under ambient light conditions in an operating room using a surgical system, the surgical system comprising a working tool including at least one optical fiber, an indicator, an optical system coupled to the working tool, and an excitation source coupled to the at least one optical fiber, the method for detecting target tissue comprising: detecting, with the optical system, fluorescence light emitted from the target tissue during a surgical procedure with the least one optical fiber of the working tool, wherein detecting the fluorescence light includes: illuminating tissue with blue light from the excitation source with the at least one optical fiber; collecting, with the at least one optical fiber, the ambient light and the fluorescence light; generating a fluorescence signal based on the fluorescence light emitted from the target tissue with the ambient light removed; determining that the target tissue is present based on the detected fluorescence light; and activating the indicator of the working tool in response to detection of the target tissue.
Clause 73—An optical probe system for determining whether brain tissue of a patient is tumorous. The optical probe system including a sample element including an optical fiber configured to transmit a fluorescence emitted by the brain tissue and an indicator configured to selectively emit visible light, the visible light being different from the fluorescence transmitted by the optical fiber. The optical probe system including an excitation source configured to emit an excitation light, the excitation light having a wavelength to induce the fluorescence in the tumorous tissue. The optical probe system including an optical instrument coupled to the optical fiber, the optical instrument configured to convert the fluorescence emitted by the brain tissue and transmitted by the optical fiber into an electrical signal; and a controller coupled to the indicator and the optical instrument, the controller configured to: determine that the brain tissue is tumorous based on the electrical signal; and activate the indicator based on the determination that the brain tissue is tumorous.
Clause 74—A neurosurgical method for differentiating brain tumor tissue from healthy brain tissue, the method comprising: providing an optical probe including a single optical fiber having a distal end; positioning the optical probe such that the optical probe contacts tissue of interest; emitting light having a wavelength from 400 to 410 nm from an excitation source through the single optical fiber to induce fluorescence emission of the tissue of interest through the single optical fiber while the optical probe is contacting tissue; receiving light emitted from the tissue of interest with the single optical fiber while the optical probe is contacting tissue; and controlling an indicator based on received light.
Clause 75—The method of clause 74, wherein the indicator is mounted to the optical probe.
Clause 76—The method of clause 74, wherein the optical probe defines a lumen, and the method further includes connecting the optical probe to a suction source.
Clause 77—A neurosurgical method for detecting tumorous tissue under ambient light conditions during a surgical procedure in an operating room, the method comprising: receiving a light source from a hand-held optical probe; analyzing the light source using a spectrometer to output a spectrometer signal; calculate a second signal by removing the effect of ambient light from the spectrometer signal; identify a single band associated with PPIX based on the second signal; and control an indicator based on the intensity of the single band.
Clause 78—A neurosurgical method for detecting tumorous tissue under ambient light conditions during a surgical procedure in an operating room, the method comprising: receiving a light source from a hand-held optical probe; analyzing the light source using a spectrometer to output a spectrometer signal; fitting a plurality of bands on the spectrometer; selecting a fitted band from the plurality of fitted bands on a mean value criterion; determining an intensity of the selected fitted band; and controlling an indicator based on the intensity of the selected fitted band.
Clause 79—A neurosurgery system for probing brain tissue of a patient for tumorous tissue, the neurosurgery system comprising: a neurosurgical tool including: a tubular member defining a lumen, an optical fiber, coupled to the tubular member; and a console including: an excitation source configured to emit an excitation light through the optical fiber, the excitation light having a wavelength to induce fluorescence in the tumorous tissue; a spectrometer configured to output a spectrometer signal; an indicator; and a controller configured to: identify a band corresponding to emission of a single fluorophore based on the spectrometer signal; determine an intensity of the identified band; and control the indicator based on the intensity of the identified band.
Clause 80—A neurosurgery system for probing brain tissue of a patient for tumorous tissue, the neurosurgery system comprising: a hand-held optical probe configured for being positioned adjacent tissue of interest; a console including: an excitation source configured to emit an excitation light through an optical fiber, the excitation light having a wavelength to induce fluorescence in the tumorous tissue; a spectrometer configured to output a spectrometer signal; an indicator; and a controller configured to: identify a band corresponding to PPIX based on the spectrometer signal; determine an intensity of the identified band; and control the indicator based on the intensity of the identified band.
Clause 81—The system of clause 80, wherein the controller is further configured to identify a second band corresponding to PPIX based on the spectrometer; determine an intensity of the identified second band; and control the indicator based on the intensity of the second identified band.
Clause 82—A neurosurgery tool assembly for differentiating brain tumor tissue from healthy brain tissue, the neurosurgery tool assembly including a neurosurgery tool including: a tubular member defining a lumen and having an outer surface; a single optical fiber coupled to the tubular member, the single optical fiber being adapted to transmit excitation light to and receive emitted light from the tissue of interest; and a light indicator coupled to the tubular member, the light indicator configured to emit light of a visible wavelength; wherein the assembly further comprises: a connector line extending from the neurosurgical tool; and a connector coupled to the connector line, the connector including a single optical fiber coupling and an electrical terminal, the connector configured to connect to a surgical console including a light source and a controller.
Clause 83—The tool assembly of clause 82, wherein the electrical terminal is coupled to the light indicator; and the single optical fiber coupling is in optical communication with a distal end of the single optical fiber.
Clause 84—The tool assembly of clause 82, further comprising a suction line, the suction line coupled a port of the tubular member, and the suction line tethered to the connector line.
Clause 85—A surgical console comprising: a connection port for receiving a connector including an optical fiber; a spectrometer; a light emitter; an optics block for routing light, the optics block defining: a first portion extending from the connection port to a first junction; an emission portion extending from the first junction to the light emitter; and a detector portion extending from the first junction to the spectrometer.
Clause 86—The surgical console of clause 85, further including a fiber lens disposed in the first portion.
Clause 87—The surgical console of clause 86, further including spectrometer lens disposed in the detector portion.
Clause 88—The surgical console of clause 85, further including a light emitter lens disposed in the emission portion.
Clause 89—The surgical console of clause 85, further including an adjustment mechanism to adjust the position of the spectrometer.
Clause 90—The surgical console of clause 85, further including an adjustment mechanism to adjust the position of the light emitter.
Clause 91—The surgical console of clause 89, wherein the adjustment mechanism includes an adjustment screw and a locking screw.
Clause 92—The surgical console of clause 89, wherein the adjustment mechanism includes a first adjustment member to adjust the position of the spectrometer in a first degree of freedom and a second adjustment member in a second degree of freedom.
Clause 93—The surgical console of clause 88, wherein the light emitter lens has a focal length different from the focal length of at least one of a spectrometer lens and a fiber lens.
Clause 94—The surgical console of clause 88, wherein the focal length of the light emitter lens has a focal length greater than the focal length of a spectrometer lens.
Clause 95—The surgical console of clause 88, wherein the focal length of the light emitter lens is greater than the focal length of a fiber lens.
Clause 96—The surgical console of clause 88, wherein the focal length of the light emitter lens is greater than both the focal length of a spectrometer lens and a fiber lens.
Clause 97—The surgical console of clause 88, further including a C-shaped member disposed in the connection port.
Clause 98—A system for differentiating tissue from tumorous tissue, the system comprising: a hand-held optical probe configured to be positioned adjacent tissue of interest; a spectrometer configured to receive collected light from the hand-held optical probe; and output a corresponding spectrometer signal; a controller in communication with the spectrometer; an indicator in communication with the controller, wherein the controller is configured to: obtain a first digital signal based on the spectrometer signal; calculate a second signal by removing the effect of ambient light from the spectrometer signal; fit a plurality of bands on the second signal; select a fitted band from the plurality of fitted bands on a mean value criterion; determine an intensity of the selected fitted band; and control the indicator based on the intensity of the selected fitted band.
Clause 99—A tool assembly for differentiating tumor tissue from healthy tissue, the tool assembly including a tool comprising: a tubular member defining a lumen and having an outer surface; a first optical fiber coupled to the tubular member, the first optical fiber being adapted to: transmit excitation light to and receive emitted light from brain tissue of interest; a second optical fiber coupled to the tubular member and configured to transmit light of a visible wavelength; a connector line extending from tool, the connecting line defining a first optical path in communication with the first optical fiber and a second optical path in communication with the second optical fiber; and a connector coupled to the connector line, the connector including a first optical fiber coupling for connecting the first optical path to a surgical console and a second fiber coupling for connecting the second optical fiber path to the surgical console.
Clause 100—A tool assembly for differentiating tumor tissue from healthy tissue, the tool assembly comprising: a hand-held probe tool comprising: a tubular member defining an outer surface; a first optical fiber coupled to the member, the first optical fiber being adapted to transmit excitation light to and receive emitted light from tissue of interest; and a second optical fiber coupled to the tubular member and configured to transmit light of a visible wavelength; a connector line extending from the hand-held probe tool, the connecting line defining a first optical path in communication with the first optical fiber and a second optical path in communication with the second optical fiber; and a connector coupled to the connector line, the connector including a first optical fiber coupling for connecting the first optical path to a surgical console and a second optical fiber coupling for connecting the second optical fiber path the surgical console.
Clause 101—An ultrasonic surgical system comprising: an ultrasonic handpiece assembly configured to remove brain tissue, the ultrasonic handpiece assembly; a sample element coupled to the ultrasonic handpiece assembly and including at least one fiber configured to collect a fluorescent light emitted from the brain tissue; an indicator fiber coupled to the ultrasonic handpiece assembly configured to selectively transmit light; a controller configured to: detect a type of brain tissue based on the fluorescent light; cause a light element to supply light to the indicator fiber based on the detected type of brain tissue; and control the ultrasonic handpiece assembly based on the detected type of brain tissue.
Clause 102—A surgical system comprising: surgical tool configured to remove brain tissue; a sample element coupled to the surgical tool and including at least one fiber configured to collect (i) a fluorescent light emitted from the brain tissue; an indicator fiber coupled to the ultrasonic handpiece assembly configured to selectively transmit light; a controller configured to: detect a type of brain tissue based on the fluorescent light; cause a light element to supply light to the indicator fiber based on the detected type of brain tissue; and control the surgical tool based on the detected type of brain tissue.
Clause 103—A surgical suction system comprising: suction tool configured to apply suction to brain tissue; a sample element coupled to the suction tool including at least one optical fiber configured to collect a fluorescent light emitted from the brain tissue; an indicator fiber coupled to the suction tool; a controller configured to: detect a type of brain tissue based on the fluorescent light; and control a light source coupled to the indicator fiber based on the detected type of brain tissue.
Clause 104—A method for detecting target tissue under ambient light conditions in an operating room during a surgical procedure, the method comprising: positioning, an optical fiber, in a sterile field that includes brain or other type of tissue being illuminated by ambient light; collecting, with the optical fiber, fluorescent light emitted from the tissue; detecting, with a controller coupled to the optical fiber, target tissue of the brain tissue or other type of tissue based on fluorescent light emitted from the brain tissue; and controlling a light source coupled to the indicator fiber positioned within the sterile field to produce a visual alert in response to the detection of the target tissue.
Clause 105—A method for detecting target tissue under ambient light conditions in an operating room using a surgical system, the surgical system comprising a working tool including at least one optical fiber, an indicator an optical system coupled to the working tool, and an excitation source coupled to the at least one optical fiber, the method for detecting target tissue comprising: positioning the working tool in a sterile field that includes tissue illuminated by ambient light; detecting, with the optical system, fluorescence light emitted from the target tissue during a surgical procedure with the least one optical fiber of the working tool, wherein detecting the fluorescence light includes: illuminating tissue with blue light from the excitation source with the at least one optical fiber; collecting, with the at least one optical fiber, the ambient light and the fluorescence light; generating a fluorescence signal based on the fluorescence light emitted from the target tissue with the ambient light removed; determining that the target tissue is present based on the detected fluorescence light; and controlling a light source coupled to the indicator fiber of the working tool in response to detection of the target tissue.
Clause 106—A method for detecting and removing target tissue under ambient light conditions in an operating room using a surgical system, the surgical system comprising a first working tool, a second working tool, an attachment including at least one optical fiber, an indicator fiber, an optical system, and an excitation source coupled to the at least one optical fiber, the method for detecting target tissue comprising: coupling the attachment to at least one of the first working tool and the second working tool; detecting, with the optical system, a target tissue during a surgical procedure based on fluorescence light emitted from the target tissue; causing the indicator fiber of the attachment to emit colored light, while at least one of the first working tool and the second working tool is in a sterile field, in response to detection of the target tissue; viewing the indicator fiber of the attachment while at least one of the first working tool and at least one of the second working tool is within the sterile field; performing a first surgical operation at a surgical site while the first working tool is in a first hand of the operator; and performing a second surgical operation at the surgical site while the second working tool is in a second hand of the operator in response to the indicator fiber's emission while maintaining the first working tool in the first hand.
Clause 107—A method for detecting target tissue under ambient light conditions in an operating room using a surgical system, the surgical system comprising a working tool including at least one optical fiber and an indicator fiber, an optical system coupled to the working tool, and an excitation source coupled to the at least one optical fiber, the method for detecting target tissue comprising: detecting, with the optical system, fluorescence light emitted from the target tissue during a surgical procedure with the least one optical fiber of the working tool, wherein detecting the fluorescence light includes: illuminating tissue with blue light from the excitation source with the at least one optical fiber; collecting, with the at least one optical fiber, the ambient light and the fluorescence light; generating a fluorescence signal based on the fluorescence light emitted from the target tissue with the ambient light removed; determining that the target tissue is present based on the detected fluorescence light; and controlling a light source coupled to the indicator fiber of the working tool in response to detection of the target tissue.
Clause 108—A neurosurgical method for differentiating brain tumor tissue from healthy brain tissue, the method comprising: providing an optical probe including a first optical fiber having a distal end; positioning the optical probe such that the optical probe contacts tissue of interest; emitting light having a wavelength from 400 to 410 nm from an excitation source through the optical fiber to induce fluorescence emission of the tissue of interest through the optical fiber while the optical probe is contacting tissue; receiving light emitted from the tissue of interest with the optical fiber while the optical probe is contacting tissue; and controlling an indicator based on received light, wherein only one fiber receives light emitted from the tissue and emits light from the excitation source.
Clause 109—A method for detecting tumorous tissue under ambient light conditions during a surgical procedure in an operating room, the method comprising: receiving a light source from a hand-held optical probe; analyzing the light source using a spectrometer to output a spectrometer signal; calculate a second signal by removing the effect of ambient light from the spectrometer signal; identify a single band associated with PPIX based on the second signal; and control a light source coupled to an indicator fiber based on the intensity of the single band.
Clause 110—A method for detecting tumorous tissue under ambient light conditions during a surgical procedure in an operating room, the method comprising: receiving collected light from a hand-held optical probe; analyzing the light source using a spectrometer to output a spectrometer signal; fitting a plurality of bands on the spectrometer; selecting a fitted band from the plurality of fitted bands on a mean value criterion; determining an intensity of the selected fitted band; and controlling a light source coupled to an indicator fiber based on the intensity of the selected fitted band or controlling an indicator based on the intensity of the selected fitted band.
Clause 111—A neurosurgery system for probing tissue of a patient for tumorous tissue, the system comprising: a tool including: a tubular member defining a lumen, an optical fiber, coupled to the tubular member; and a console including: an excitation source configured to emit an excitation light through the optical fiber, the excitation light having a wavelength to induce fluorescence in the tumorous tissue; a spectrometer configured to output a spectrometer signal; an indicator fiber coupled to the tool; and a controller configured to: identify a band corresponding to emission of a single fluorophore based on the spectrometer signal; determine an intensity of the identified band; and control a light source coupled to the indicator fiber based on the intensity of the identified band.
Clause 112—A method for detecting tumorous tissue under ambient light conditions during a surgical procedure in an operating room, the method comprising: receiving a light source from a hand-held optical probe; analyzing the light source using a spectrometer to output a spectrometer signal; calculate a second signal by removing the effect of ambient light from the spectrometer signal; identify a single band associated with PPIX based on the second signal; and control a light coupled to an optical fiber based on the intensity of the single band.
Clause 113—A system for probing tissue of a patient for tumorous tissue, the system comprising: a tool, an optical fiber coupled to the tool; and a console including: an excitation source configured to emit an excitation light through the optical fiber, the excitation light having a wavelength to induce fluorescence in the tumorous tissue; a spectrometer configured to output a spectrometer signal; an indicator fiber coupled to the tool; and a controller configured to: identify a band corresponding to emission of a single fluorophore based on the spectrometer signal; determine an intensity of the identified band; and control the a light source coupled to the indicator fiber based on the intensity of the identified band.
Clause 114—A tool assembly for differentiating tumor tissue from healthy tissue, the tool assembly including a neurosurgery tool including: a tubular member defining a lumen and having an outer surface; an optical fiber coupled to the tubular member, the optical fiber being adapted to transmit excitation light to and receive emitted light from the tissue of interest; and an indicator fiber coupled to the tubular member, the indicator fiber coupled to a light source configured to emit light of a visible wavelength; wherein the assembly further comprises: a connector line extending from the neurosurgical tool; and a connector coupled to the connector line, the connector including two optical fiber couplings, the connector configured to connect to a surgical console including an indicator light source and an emission light source and a controller.
Clause 115—A neurosurgery system for differentiating healthy brain tissue from tumorous brain tissue, the neurosurgery system comprising: a hand-held optical probe configured to be positioned adjacent tissue of interest; a spectrometer configured to receive collected light from the hand-held optical probe and output a corresponding spectrometer signal; a controller in communication with the spectrometer; an indicator in communication with the controller, wherein the controller is configured to: obtain a first digital signal based on the spectrometer signal; calculate a second signal by removing the effect of ambient light from the spectrometer signal; fit a plurality of bands on the second signal; select a fitted band from the plurality of fitted bands on a mean value criterion; determine an intensity of the selected fitted band; and control the indicator based on the intensity of the selected fitted band.
Clause 116—The neurosurgery system of clause 115, wherein the step of fitting a plurality of bands on the second signal is further defined as fitting a plurality of distribution curves on the second signal.
Clause 117—The neurosurgery system of clause 116, wherein the plurality of distribution curves are further defined as a plurality of gaussian distribution curves, a plurality of Lorentzian distribution curves, or a combination thereof.
Clause 118—The neurosurgery system of clause 115, wherein the controller is further configured to select a fitted band that exhibits a mean value criterion of 635 nm.
Clause 119—The neurosurgery system of clause 118, wherein the controller is further configured to select a fitted band from the plurality of fitted bands based on the mean value criterion and a standard deviation criterion.
Clause 120—The neurosurgery system of clause 115, wherein the controller is further configured to select a fitted band from the plurality of fitted bands based on a standard deviation criterion value.
Clause 121—The neurosurgery system of clause 115, wherein the controller is further configured to control the indicator based on the intensity of the selected fitted band and a predetermined intensity threshold.
Clause 122—The neurosurgery system of clause 119, wherein the controller is further configured to calculate an amplitude offset based on an amplitude of the second signal and the selected fitted band and to control the indicator based on the amplitude offset and a predetermined amplitude offset threshold.
Clause 123—The neurosurgery system of clause 122, wherein the controller is further configured to calculate the amplitude offset based on the center of the selected fitted band.
Clause 124—The neurosurgery system of clause 115, wherein the controller is further configured to calculate the second signal by fitting a baseline polynomial curve to the spectrometer signal and subtract the baseline polynomial curve from the spectrometer signal to remove artifacts of ambient light to yield the second signal.
Clause 125—The neurosurgery system of clause 124, wherein system is further configured to fit the baseline polynomial curve to a region of the spectrometer signal.
Clause 126—A neurosurgery tool assembly for differentiating brain tumor tissue from healthy brain tissue, the neurosurgery tool assembly including a neurosurgery tool comprising: a tubular member defining a lumen and having an outer surface; a first optical fiber coupled to the tubular member, the first optical fiber being adapted to transmit excitation light to and receive emitted light from brain tissue of interest; a second optical fiber coupled to the tubular member and configured to transmit light of a visible wavelength; a connector line extending from the neurosurgery tool, the connecting line defining a first optical path in communication with the first optical fiber and a second optical path in communication with the second optical fiber; and a connector coupled to the connector line, the connector including a first optical fiber coupling for connecting the first optical path to a surgical console and a second fiber coupling for connecting the second optical fiber path to the surgical console.
Clause 127—The neurosurgery tool assembly of clause 126, wherein the outer surface of the tubular member defines a first channel, the first optical fiber being at least partially disposed within the first channel, wherein the neurosurgery tool further includes a sleeve surrounding a portion of the first optical fiber and the tubular member.
Clause 128—The neurosurgery tool assembly of clause 127, wherein the second optical fiber is at least partially disposed within the first channel and the sleeve surrounds a portion of the first optical fiber, the second optical fiber, and the tubular member.
Clause 129—The neurosurgery tool assembly of clause 127, wherein the outer surface of the tubular member defines a second channel separate from the first channel, the second optical fiber being at least partially disposed within the second channel.
Clause 130—The neurosurgery tool assembly of clause 126, wherein the neurosurgery tool assembly is free from an electrical terminal.
Clause 131—The neurosurgery tool assembly of clause 126, wherein the first optical fiber has a diameter of less than 500 μm.
Clause 132—The neurosurgery tool assembly of clause 126, wherein a distal end of the first optical fiber is arranged proximate a distal end of the tubular member, and a distal end of the second optical fiber is arranged proximally of the distal end of the first optical fiber.
Clause 133—The neurosurgery tool assembly of clause 132, wherein the first optical fiber and the second optical fiber are arranged adjacent to each other.
Clause 134—The neurosurgery tool assembly of clause 126, wherein a distal end of the first optical fiber is aligned with a distal end of the tubular member.
Clause 135—The neurosurgery tool assembly of clause 126, wherein a distal end of the first optical fiber is arranged on a first side of the tubular member, and wherein a distal end of the second optical fiber is arranged on a second side of the tubular member opposite of the first side.
Clause 136—The neurosurgery tool assembly of clause 126, wherein the tubular member is curved along its length in a first direction, wherein a distal end of the first optical fiber is arranged on a side of the tubular member in a direction of curvature, and a distal end of the second optical fiber is arranged on a side of the tubular member opposite the direction of curvature.
Clause 137—The neurosurgery tool assembly of clause 126, further comprising a distal tube sleeve arranged on an outer surface of the tubular member and at a distal end of the tubular member, the distal tube sleeve comprising at least partially transparent material.
Clause 138—The neurosurgery tool assembly of clause 137, further comprising a proximal tube sleeve, arranged on a portion of the outer surface of the tubular member proximal of the distal tube sleeve, the proximal tube sleeve comprising an opaque material.
Clause 139—The neurosurgery tool assembly of clause 126, wherein a portion of the second optical fiber comprises a textured surface for diffusing transmitted light.
Clause 140—The neurosurgery tool assembly of clause 139, wherein the outer surface of the second optical fiber is deformed via a crimping process to form the textured surface.
Clause 141—A neurosurgery system for differentiating brain tumor tissue from healthy brain tissue, the neurosurgery system comprising: a hand-held optical probe configured to be positioned adjacent to tissue of interest; a spectrometer configured to receive collected light from the hand-held optical probe and output a corresponding spectrometer signal; a controller in communication with the spectrometer, wherein the controller is configured to: calculate a second signal by removing the effect of ambient light from the spectrometer signal; identify a single band associated with PPIX based on the second signal; and control an indicator based on the intensity of the single band.
Clause 142—The neurosurgery system of clause 141, wherein the indicator comprises a visual indicator configured to emit light when the intensity of the single band is above a predetermined threshold.
Clause 143—The neurosurgery system of clause 142, wherein the indicator further comprises an audible indicator configured to emit sound when the intensity of the single band is above a predetermined threshold.
Clause 144—The neurosurgery system of clause 142, wherein the indicator is further configured to emit light at a brightness corresponding to the intensity of the single band.
Clause 145—The neurosurgery system of clause 141, wherein the indicator comprises an audible indicator configured to emit sound when the intensity of the single band is above a predetermined threshold.
Clause 146—The neurosurgery system of clause 141, wherein the indicator is coupled to the hand-held optical probe.
Clause 147—The neurosurgery system of clause 146, wherein the indicator is arranged adjacent to a light collecting portion of the hand-held optical probe.
Clause 148—A method of forming a hand-held surgical probe including an optical fiber for providing indications to a user, the method comprising: providing a suction tool body defining a lumen and having an outer surface; positioning a mandrel adjacent the suction tool body; positioning a first heat shrink tube to partially encompass the suction tool body and the mandrel; applying heat to the first heat shrink tube to form a first deformed heat shrink tube to encompass a portion of the suction tool body; removing the mandrel from being adjacent the suction tool body; routing an optical fiber between the suction tool body and an inner diameter of the first deformed heat shrink tube; and positioning a second deformed heat shrink tube over a distal end of the suction tool body partially encompass a portion of the suction tool body and the optical fiber.
Clause 149—The method of clause 148, further comprising pre-forming the second heat shrink tube to form the second deformed heat shrink tube before positioning the second heat shrink tube over the distal end of the suction tool body.
Clause 150—The method of clause 149, wherein the step of pre-forming comprises applying heat to the second heat shrink tube while the second heat shrink tube is positioned about a second mandrel having a shape matching a shape of a distal end portion of the suction tool body.
Clause 151—The method of clause 148, wherein the second deformed heat shrink tube is formed from a transparent material.
Clause 152—The method of clause 148, wherein the first heat shrink tube is formed from an opaque material.
Clause 153—The method of clause 148, wherein the optical fiber has a melting point that is lower than the melting point of first heat shrink tube and the second heat shrink tube.
Clause 154—The method of clause 148, further comprising securing the second deformed heat shrink tube to the suction tool body using an adhesive.
Clause 155—The method of clause 154, wherein the adhesive is UV curable.
Clause 156—The method of clause 148, further positioning the optical fiber in a groove formed in an outer diameter of the suction tool body.
Clause 157—The method of clause 156, wherein the step of positioning the second deformed heat shrink tube comprises positioning the second deformed heat shrink tube such that the second deformed heat shrink tube partially surrounds the groove.
Clause 158—The method of clause 152, wherein the melting point of the optical fiber is at least 50 degrees lower than the melting point of the first heat shrink.
Clause 159—The method of clause 148, wherein a thickness of the second deformed heat shrink tube is less than the thickness of the first deformed heat shrink tube.
Clause 160—The method of clause 159, wherein the thickness of the second deformed heat shrink tube is at least 75% less than the thickness of the first deformed heat shrink tube.
Clause 161—The method of clause 148, wherein a thickness of the second deformed heat shrink tube is less than 2% of an outer diameter of the distal end of the suction tool body.
Clause 162—A surgical method of assessing a skull-base tumor of a patient who was administered a fluorescing agent, the method comprising: providing a tissue detection system including an optical system and a surgical instrument, the surgical instrument including at least optical fiber extending to a distal end of the surgical instrument for transmitting excitation light and for receiving fluorescent emissions from the skull-base tumor; creating a hole through a proximal and distal wall of a sphenoid bone of a skull of the patient; inserting the surgical instrument trans-nasally through the proximal and distal walls of the sphenoid bone such that the distal end of the optical fiber is positioned in contact with the skull-base tumor of the patient; and controlling an indicator of the tissue detection system based on the fluorescent emissions from the skull-base tumor.
Clause 163—The surgical method of clause 162, wherein the skull-base tumor is a pituitary tumor.
Clause 164—The surgical method of clause 162, wherein the indicator is visible outside of the skull of the patient.
Clause 165—The surgical method of clause 162, further comprising: inserting an endoscope trans-nasally through the proximal and distal wall of the sphenoid bone such that a distal end of the endoscope is positioned adjacent the skull-base tumor of the patient.
Clause 166—The surgical method of clause 165, wherein the surgical instrument is inserted through a first nostril of the patient and the endoscope is inserted through a second nostril of the patient.
Clause 167—The surgical method of clause 165, wherein the endoscope is coupled to a display and the display includes the indicator.
Clause 168—The surgical method of clause 167, wherein the display includes a graphical user interface and the graphical user interface includes the indicator.
Clause 169—The surgical method of clause 162, wherein the indicator is coupled to the surgical instrument.
Clause 170—The surgical method of clause 169, wherein: the tissue detection system includes a controller in communication with the optical system and the indicator; the indicator includes an optical fiber connected to a light source; and the light source is controlled by the controller based on the fluorescent emissions from the skull-base tumor.
Clause 171—The surgical method of clause 162, wherein a shaft of the surgical instrument includes at least one bend.
Clause 172—The surgical method of clause 162, wherein the surgical instrument is an ultrasonic resection tool or bipolar forceps.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the examples is described above as having certain features, any one or more of those features described with respect to any example of the disclosure can be implemented in and/or combined with features of any of the other examples, even if that combination is not explicitly described. In other words, the described examples are not mutually exclusive, and permutations of one or more examples with one another remain within the scope of this disclosure.

Spatial and functional relationships between elements (for example, between controllers, circuit elements, semiconductor layers, etc.) are described using various terms, including “connected”, “engaged”, “coupled”, “adjacent”, “next to”, “on top of”, “above”, “below”, and “disposed”. Unless explicitly described as being “direct”, when a relationship between first and second elements is described in the above disclosure, that relationship can be a direct relationship where no other intervening elements are present between the first and second elements but can also be an indirect relationship where one or more intervening elements are present (either spatially or functionally) between the first and second elements.

As used herein, the phrase at least one of A, B, and C should be construed to mean a logical (A OR B OR C), using a non-exclusive logical OR, and should not be construed to mean “at least one of A, at least one of B, and at least one of C.” The term subset does not necessarily require a proper subset. In other words, a first subset of a first set may be coextensive with (equal to) the first set.

In the figures, the direction of an arrow, as indicated by the arrowhead, generally demonstrates the flow of information (such as data or instructions) that is of interest to the illustration. For example, when element A and element B exchange a variety of information, but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A.

In this application, including the definitions below, the term “controller” or “module” may be replaced with the term “circuit”. The term “controller” may refer to, be part of, or include: an Application Specific Integrated Circuit (ASIC); a programmable system on a chip (PSoC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The controller may include one or more interface circuits with one or more transceivers. In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 802.11-2016 (also known as the WIFI wireless networking standard) and IEEE Standard 802.3-2015 (also known as the ETHERNET wired networking standard). Examples of a WPAN are the BLUETOOTH wireless networking standard from the Bluetooth Special Interest Group and IEEE Standard 802.15.4.

The controller may communicate with other controllers using the interface circuit(s). Although the controller may be depicted in the present disclosure as logically communicating directly with other controllers, in various implementations the controller may actually communicate via a communications system. The communications system may include physical and/or virtual networking equipment such as hubs, switches, routers, gateways, and transceivers. In some implementations, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In various implementations, the functionality of the controller may be distributed among multiple controllers that are connected via the communications system. For example, multiple controllers may implement the same functionality distributed by a load balancing system. In a further example, the functionality of the controller may be split between a server (also known as remote, or cloud) controller and a client (or, user) controller.

Some or all hardware features of a controller may be defined using a language for hardware description, such as IEEE Standard 1364-2005 (commonly called “Verilog”) and IEEE Standard 1076-2008 (commonly called “VHDL”). The hardware description language may be used to manufacture and/or program a hardware circuit. In some implementations, some or all features of a controller may be defined by a language, such as IEEE 1666-2005 (commonly called “SystemC”), that encompasses both code, as described below, and hardware description.

The term code, as used above, may include software, firmware, and/or microcode, and may refer to programs, routines, functions, classes, data structures, and/or objects. The term shared processor circuit encompasses a single processor circuit that executes some or all code from multiple controllers. The term group processor circuit encompasses a processor circuit that, in combination with additional processor circuits, executes some or all code from one or more controllers. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple controllers. The term group memory circuit encompasses a memory circuit that, in combination with additional memories, stores some or all code from one or more controllers.

The term memory circuit is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general-purpose computer to execute one or more particular functions embodied in computer programs. The functional blocks and flowchart elements described above may serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, ObjectiveC, Swift, Haskell, Go, SQL, R, Lisp, Java®, Fortran, Perl, Pascal, Curl, OCaml, JavaScript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

What is claimed is:

1. A surgical method of assessing tumor margins of obscured tissue of a patient who was administered a fluorescing agent, the method comprising:
providing a tissue detection system including an optical system and a surgical instrument for resecting tissue, the surgical instrument including at least one optical fiber extending to a distal end of the surgical instrument for transmitting excitation light and for receiving fluorescent emissions from the obscured tissue, a handle portion, and a shaft extending from the handle portion;
providing a surgical microscope which has a field of view;
creating an access hole having a first width through a tissue of the patient and extending to a surgical site of the patient;
accessing the surgical site through the access hole and resecting tissue at the surgical site of the patient with the surgical instrument to create a resection cavity having a second width which is larger than the first width and at least a portion of the resection cavity being outside of the field of view of the surgical microscope and including the obscured tissue;
positioning the surgical instrument through the access hole and within the resection cavity such that the surgical instrument is in contact with the obscured tissue;
collecting fluorescent emission from the obscured tissue using the at least one optical fiber of the surgical instrument; and
controlling an indicator of the tissue detection system based on the fluorescent emissions from the obscured tissue as collected by the at least one optical fiber of the surgical instrument.

2. The surgical method of claim 1, wherein:
the resection cavity includes a first resection volume and a second resection volume;
the second resection volume is outside of the field of view of the surgical microscope and includes the obscured tissue; and
positioning the surgical instrument in contact with the obscured tissue includes positioning the surgical instrument at least partially outside of the field of view of the surgical microscope and in contact with a portion of the second resection volume.

3. The surgical method of claim 1, wherein the surgical instrument is a suction tool configured to couple to a vacuum source and apply a suction force to the surgical site.

4. The surgical method of claim 3, wherein the suction tool includes an aperture for controlling the suction force (i) manually and/or (ii) in response to the indicator of the tissue detection system.

5. The surgical method of claim 3, wherein the surgical suction tool is operable in multiple modes including:
an observation mode in which the surgical suction tool is configured to detect fluorescent emission from the obscured tissue without applying the suction force; and
a resection mode in which the surgical suction tool is configured to detect fluorescent emission from the obscured tissue while applying the suction force.

6. The surgical method of claim 3, wherein the surgical suction tool is operable in a manual detection mode in which the indicator of the surgical suction tool is disabled and does not respond to detections of fluorescent emission from tissue.

7. The surgical method of claim 5, further comprising:
maintaining the surgical suction tool in the observation mode; and
switching the surgical suction tool from the observation mode to the resection mode based on the fluorescent emission from the obscured tissue as collected by the optical fiber of the surgical suction tool.

8. The surgical method of claim 1, further comprising:
positioning the surgical instrument in contact with a first portion of the obscured tissue;
detecting fluorescent emission from the first portion of the obscured tissue and resecting the first portion of the obscured tissue with the surgical instrument in response to the detection;
determining that the optical fiber of the surgical instrument does not detect fluorescent emissions and disabling a resection function of the surgical instrument in response to the determination;
moving the surgical instrument within the resection cavity to a position which is in contact with a second portion of the obscured tissue;
detecting fluorescent emission from the second portion of the obscured tissue; and
enabling the resection function of the surgical instrument in response to the detection;
resecting the second portion of the obscured tissue with the surgical instrument.

9. The surgical method of claim 1, further comprising:
resecting a first portion of the obscured tissue with the surgical instrument;
determining that the optical fiber of the surgical instrument no longer detects fluorescent emissions;
displacing tissue without resecting the tissue;
positioning the distal end of the surgical instrument in contact with a second portion of the obscured tissue while displacing tissue within the resection cavity; and
resecting the first portion of the obscured tissue.

10. The surgical method of claim 1, wherein the indicator is coupled to the surgical instrument.

11. The surgical method of claim 10, wherein:
the tissue detection system includes a controller in communication with the optical system and the indicator;
the indicator includes an optical fiber connected to a light source; and
the light source is controlled by the controller based on the fluorescent emissions from the obscured tissue as collected by the at least one optical fiber of the surgical instrument.

12. The surgical method of claim 1, wherein the shaft of the surgical instrument includes at least one bend.

13. The surgical method of claim 1, wherein the surgical instrument is an ultrasonic resection tool or bipolar forceps.

14. The surgical method of claim 1, wherein the resection cavity is illuminated with a light color which is different from the excitation light emitted from the at least one optical fiber.

15. The surgical method of claim 1, wherein the indicator includes a reposition indicator configured to indicate to a user that the surgical microscope should be repositioned to change the field of view of the surgical microscope to cause at least a portion of the obscured tissue to be within the field of view.

16. The surgical method of claim 1, wherein
positioning the surgical instrument through the access hole and within the resection cavity obscures at least a portion of the field of view of the surgical microscope; and
the obscured portion of the field of view includes shadowed tissue.

17. The surgical method of claim 16, further comprising:
positioning the surgical instrument such that the surgical instrument is in contact with the shadowed tissue;
collecting fluorescent emission from the shadowed tissue using the at least one optical fiber of the surgical instrument; and
controlling the indicator of the tissue detection system based on the fluorescent emissions from the shadowed tissue as collected by the at least one optical fiber of the surgical instrument.

18. The surgical method of claim 16, further comprising:
positioning the surgical instrument such that at least a portion of the shadowed tissue is illuminated by the excitation light from the at least one optical fiber of the tissue detection system; and
controlling the indicator of the tissue detection system based on the fluorescent emissions from the shadowed tissue.

19. The surgical method of claim 16, further comprising:
positioning the surgical instrument such that at least a portion of the shadowed tissue is illuminated by the excitation light from the at least one optical fiber of the tissue detection system; and
disabling the indicator of the tissue detection system to allow the surgeon to observe the fluorescent emissions from the shadowed tissue without interference from light emitted by the indicator.

20. A surgical method of assessing a skull-base tumor of a patient who was administered a fluorescing agent, the method comprising:
providing a tissue detection system including an optical system and a surgical instrument, the surgical instrument including at least optical fiber extending to a distal end of the surgical instrument for transmitting excitation light and for receiving fluorescent emissions from the skull-base tumor;
creating a hole through a proximal and distal wall of a sphenoid bone of a skull of the patient;
inserting the surgical instrument trans-nasally through the proximal and distal walls of the sphenoid bone such that the distal end of the optical fiber is positioned in contact with the skull-base tumor of the patient; and
controlling an indicator of the tissue detection system based on the fluorescent emissions from the skull-base tumor.
